(19) United States
(12) Patent Application Publication
(10) Pub. No.: US 2023/0272348 A1
(43) Pub. Date: Aug. 31, 2023

(54) METHODS OF GENERATING HEMATOPOIETIC CELL PREPARATIONS

(71) Applicant: The Penn State Research Foundation, University Park, PA (US)

(72) Inventors: Xiaojun Lian, State College, PA (US); Lauren Nicole Randolph, State College, PA (US); Yuqian Jiang, State College, PA (US)

(21) Appl. No.: 18/012,024

(22) PCT Filed: Jun. 22, 2021
(86) PCT No.: PCT/US21/38450

§ 371 Date: Dec. 21, 2022

Related U.S. Application Data
(60) Provisional application No. 63/042,071, filed on Jun. 22, 2020.
(60) Provisional application No. 63/152,605, filed on Feb. 23, 2021.

(51) Int. Cl. C12N 5/071; C12N 15/90

(52) U.S. Cl. CPC C12N 5/0692; C12N 15/907; C12N 2506/45; C12N 2501/727; C12N 2501/165

(57) ABSTRACT

The present invention relates to a method of producing an enriched preparation of hemogenic endothelial progenitor cells. This method involves providing a population of pluripotent stem cells and inducing expression of a SOXF transcription factor in the pluripotent stem cells of the population. The method further involves culturing the population of pluripotent stem cells expressing the SOXF transcription factor, whereby the enriched preparation of hemogenic endothelial progenitor cells is produced as a result of said culturing. The present invention also relates to a method of producing an enriched preparation of hematopoietic progenitor cells and methods of treating a subject having a condition mediated by a loss or dysfunction of hematopoietic stem cells or by a loss of immune cells.

### CROSS REFERENCE TO RELATED APPLICATIONS

This application claims the priority benefit of U.S. Provisional Patent Application Ser. No. 63/152,605, filed Feb. 23, 2021, and U.S. Provisional Patent Application Ser. No. 63/042,071, filed Jun. 22, 2020, which are hereby incorporated by reference in their entirety.

### STATEMENT REGARDING FEDERALLY SPONSORED RESEARCH OR DEVELOPMENT

This invention was made with government support under Grant No. EB026035 awarded by the National Institutes of Health and under Grant No. CBET1943696 awarded by the National Science Foundation. The Government has certain rights in the invention.

### FIELD

The present disclosure relates to methods of generating enriched preparations of hemogenic endothelial cells and hematopoietic progenitor cells. The disclosure also relates to enriched preparations of hemogenic endothelial cells and hematopoietic progenitor cells generated in accordance with the methods described herein and their use in treating a subject having a condition mediated by a loss of hematopoietic stem cells and immune cells.

### BACKGROUND

Human pluripotent stem cell (hPSC) differentiation via growth factors and/or small molecules often results in heterogeneous populations of cells, dramatically affecting our ability to efficiently derive therapeutically relevant cell types (X. Lian, et al., “Efficient Differentiation of Human Pluripotent Stem Cells to Endothelial Progenitors via Small-Molecule Activation of WNT Signaling,” Stem Cell Reports 3:804-816 (2014); K.-D. Choi, et al., “Identification of the hemogenic endothelial progenitor and its direct precursor in human pluripotent stem cell differentiation cultures,” Cell Rep. 2:553-567 (2012); A. Ditadi, et al., “Human definitive haemogenic endothelium and arterial vascular endothelium represent distinct lineages,” Nat. Cell Biol. 17:580-91 (2015); F. W. Pagliuca, et al., “Generation of functional human pancreatic β cells in vitro,” Cell 159:428-439 (2014)). Generally speaking, a somatic cell, which emerges late in human development, is more difficult to derive because the mechanistic basis of that cell's development may be largely unknown. For example, hematopoietic development occurs at several developmental timepoints thereby increasing the complexity. Primitive hematopoiesis is defined as occurring in the yolk sac and does not give rise to lymphoid cells or hematopoietic stem cells (HSCs) (M. Ackermann et al., “Lost in translation: pluripotent stem cell-derived hematopoiesis,” EMBO Mol. Med 7:1388-402 (2015)). A second hematopoietic event localized in the yolk sack produces erythroid-myeloid progenitors as well as lymphoid progenitor cells (M. Ackermann et al., “Lost in translation: pluripotent stem cell-derived hematopoiesis,” EMBO Mol. Med 7:1388-402 (2015)). Despite closer resemblance to adult blood components, this hematopoietic event does not produce definitive HSCs (M. Ackermann et al., “Lost in translation: pluripotent stem cell-derived hematopoiesis,” EMBO Mol. Med 7:1388-402 (2015)). Chronologically the third stage of hematopoiesis in mammals is the first to generate HSCs capable of long-term, multi-lineage blood reconstitution and is thus termed definitive hematopoiesis (A. Ditadi et al., “A view of human haematopoietic development from the Petri dish,” Nat. Rev. Mol. Cell Biol. 18:56-67 (2017)). Definitive hematopoietic cells develop from hemogenic endothelial (HE) progenitors via an endothelial-to-hematopoietic transition (EHT) process (M. Ackermann et al., “Lost in translation: pluripotent stem cell-derived hematopoiesis,” EMBO Mol. Med 7:1388-402 (2015); A. Ditadi et al., “A view of human haematopoietic development from the Petri dish,” Nat. Rev. Mol. Cell Biol. 18:56-67 (2017); A. Ivanovs, et al., “Human haematopoietic stem cell development: From the embryo to the dish,” Development 144:2323-2337 (2017)). Due to the lack of complete understanding of molecular pathways controlling definitive HSC development, it has been challenging to efficiently derive HE progenitor cells from hPSCs.

An alternative method to derive desired cell types from hPSCs is forward programming, which is defined by overexpression of appropriate transcription factors (TFs) in hPSCs to differentiate them into desired cell types. Forward programming can generate desired cells with a high efficiency in a much shorter period of time. For example, NFIA overexpression is sufficient to generate astrocytes from neural stem cells within 3 weeks, as compared to 3-6 months using growth factor/small molecule based protocols (J. Tchieu, et al., “NFIA is a gliogenic switch enabling rapid derivation of functional human astrocytes from pluripotent stem cells,” Nat. Biotechnol. 37:267-275 (2019)). To apply forward programming to derive HE cells, it is critical to know which TFs are specifically expressed in this population.

With the development of single cell analysis techniques, researchers are able to characterize differentiated cell types at a depth previously unattainable. Notably, single-cell RNA sequencing (scRNA-seq) analysis reported heterogeneity in many differentiated populations, including endothelial cells and pancreatic beta cells (D. T. Paik, et al., Large-scale single-cell RNA-seq reveals molecular signatures of heterogeneous populations of human induced pluripotent stem cell-derived endothelial cells,” Circ. Res. 123:443-450 (2018), A. Veres, et al., “Charting cellular identity during human in vitro β-cell differentiation,” Nature 569:368-373 (2019)). It follows that scRNA-seq can be used to identify critical TFs solely expressed in desired cell populations, which may be used for forward programming.

The present disclosure is directed to overcoming these and other deficiencies in the art.

### SUMMARY

One aspect of the disclosure relates to a method of producing an enriched preparation of hemogenic endothelial progenitor cells. This method involves providing a population of pluripotent stem cells and inducing expression of a SOXF transcription factor in the pluripotent stem cells of the population. The method further involves culturing the population of pluripotent stem cells expressing the SOXF transcription factor, whereby the enriched preparation of hemogenic endothelial progenitor cells is produced as a result of said culturing.

Another aspect of the present disclosure relates to a preparation of hemogenic endothelial progenitor cells produced in accordance with the methods described herein, and preparations of cells enriched for the produced hemogenic endothelial progenitor cells.

Another aspect of the present disclosure relates to methods of treating a subject having a condition mediated by a loss or dysfunction of hematopoietic stem cells, i.e., a subject in need of hematopoietic reconstitution. This method involves administering, to the subject having a condition mediated by a loss of hematopoietic stem cells, the enriched preparation of hemogenic endothelial progenitor cells, or a preparation of cells differentiated from said enriched preparation of hemogenic endothelial cells under conditions effective to treat the condition.

Another aspect of the present disclosure is directed to a method of producing an enriched preparation of hematopoietic progenitor cells. This method involves providing a population of pluripotent stem cells, and inducing expression of a SOXF transcription factor in pluripotent stem cells of the population, wherein an enriched population of hemogenic endothelial progenitor cells is produced as a result of said inducing. The method further involves discontinuing SOXF transcription factor expression in the population of hemogenic endothelial progenitor cells and culturing the population of hemogenic endothelial progenitor cells under conditions effective to produce an enriched preparation of hematopoietic progenitor cells.

Another aspect of the present disclosure relates to a preparation of hematopoietic progenitor cells produced in accordance with the methods described herein, and preparations of cells enriched for the produced hematopoietic progenitor cell.

Another aspect of the present disclosure relates to a method of treating a subject having a condition mediated by a loss of immune cells, e.g., a subject needing immune cell reconstitution. This method involves administering to the subject the enriched preparation of hematopoietic progenitor cells as described herein under conditions effective to treat the condition.

Another aspect of the present disclosure relates to a preparation of human cells derived from a pluripotent stem cell line, wherein at least 60% of the preparation comprises hemogenic endothelial progenitor cells expressing VE-cadherin (VEC) and CD34 but do not express CD31.

Another aspect of the present disclosure relates to a kit, where the kit includes reagents suitable for differentiating pluripotent stem cells into hemogenic endothelial progenitor cells and/or hematopoietic stem cells. In some embodiments, the kit comprises a nucleic acid molecule encoding a SOXF transcription factor (e.g., SOX7, SOX17, SOX18, or any combination thereof) and reagents suitable for transfecting a preparation of pluripotent stem cells with said SOXF transcription factor nucleic acid molecule.

Another aspect of the present disclosure relates to a recombinant nucleic acid construct comprising the nucleotide sequence of SEQ ID NO: 3 (SOX17) or a nucleotide sequence having at least 85% sequence identity to the nucleotide sequence of SEQ ID NO: 3.

Transcription factors (TFs) play critical roles in stem cell maintenance and differentiation. Using single cell RNA sequencing, TFs expressed in hemogenic endothelial (HE) progenitors differentiated from human pluripotent stem cells (hPSCs) were investigated and upregulated expression of SOXF factors SOX7, SOX17, and SOX18 in the HE population was identified. To test whether overexpression of these factors increases HE differentiation efficiency, inducible hPSC lines were established and only SOX17 improved differentiation. Temporal expression analysis further revealed SOX17 was turned on immediately before VE-Cadherin, indicating SOX17 may be a causative factor for HE differentiation. Upon SOX17 knockdown via CRISPR-Cas13d, HE differentiation was significantly abrogated. Strikingly, it was discovered that SOX17 overexpression alone is sufficient to generate more than 50% CD34+VE−cadherin+CD73− cells that could be directed to hematopoietic progenitors, which emerged via an endothelial-to-hematopoietic transition and significantly upregulated definitive hematopoietic transcriptional programs. Functional assays showed that these progenitors can differentiate into blood cells from multiple lineages. These analyses reveal an uncharacterized function of SOX17 in directing hPSCs differentiation towards HE cells.

Using scRNA-seq analysis, it was revealed that SOXF factors (V. Lefebvre et al., “Control of cell fate and differentiation by Sry-related high-mobility-group box (Sox) transcription factors,” Int. J. Biochem. Cell Biol. 39:2195-2214 (2007), which is hereby incorporated by reference in its entirety), SOX17, SOX7, and SOX18, are specifically expressed in hPSC derived endothelial progenitors. SOX17 was systematically studied during hPSC differentiation to HE progenitors and it was illustrated that SOX17 overexpression enhances HE differentiation. Knockdown of SOX17 via CRISPR-Cas13d, however, inhibited HE progenitor differentiation. Importantly, overexpression of SOX17 alone in the absence of any small molecules or growth factors is sufficient to differentiate hPSCs into HE progenitors that can further differentiate into multiple hematopoietic lineages. In summary, these findings provide new insights into HE development and point out a critical role of SOX17 in differentiating hPSCs into HE cells, enabling a novel forward programming method to generate HE cells via overexpression of SOX17 in hPSC.

Hemogenic endothelial (HE) cells have been generated from human pluripotent stem cells (hPSCs) to study blood development. However, their full transcriptomic characterization and key genes involving in directing HE differentiation is unclear. Utilizing single cell RNA-seq analysis, it was found that SOX17 is solely expressed in HE cells and is also required for HE differentiation. Strikingly, overexpression of SOX17 alone was found sufficient to program hPSCs into CD34+VE-cadherin+CD73⁻ HE cells, which could further differentiate into blood progenitors. This research reveals that SOX17 is sufficient to direct hPSCs differentiation to HE cells.

### BRIEF DESCRIPTION OF THE DRAWINGS

FIGS. 1A-1I show that CH-induced endothelial progenitor differentiation method yields population expressing hemogenic endothelial markers and SOXF family. FIG. 1A is a schematic of endothelial progenitor differentiation used to test for hemogenic potential. FIG. 1B is a graph showing UMAP dimensional reduction projection showing clustering of cells produced on D5. FIG. 1C is a heat map indicating the top 10 variably expressed genes for each cluster. FIG. 1D shows violin plots identifying CD34, VEC, and CD31 expression in cluster 1. FIG. 1E shows SOXF family member expression in D5 population. FIGS. 1F and 1G are immunofluorescence images of day 5 endothelial progenitors derived from SOX17-mCherry reporter cells. mCherry expression is seen in cells that express VEC (FIG. 1F) and CD31 (FIG. 1G). Nuclear staining was done with Hoechst 33342. Scale bars are 100 μm. FIG. 1H shows immunofluorescence images showing VEC and SOX17 co-expression in day 5 cells differentiated from 6-9-9 cells. White boxes indicate locations of enlarged views with symbols indicating the corresponding image. Nuclear staining was done with Hoechst 33342. Left two scale bars are 100 μm and right two scale bars are 50 μm. FIG. 1I shows flow cytometry analysis showing co-expression of SOX17 and VEC and SOX17 and CD34 in day 5 cells.

FIGS. 2A-2Q show single cell RNA sequencing data analysis and validation. FIG. 2A shows Violin plots indicating the number of genes, counts, and percentage of the reads mapped to mitochondrial genes. FIG. 2B shows a heat map for principal component 1. FIG. 2C is a Jack Straw plot for the first 50 principal components. FIG. 2D is an Elbow plot for the first 50 principal components. FIGS. 2E-2J show Violin plots showing expression of HE cell associated genes CD93 (FIG. 2E), MECOM (FIG. 2F), ETS1 (FIG. 2G), KDR (FIG. 2H), KIT (FIG. 2I), and RUNX1 (FIG. 2J). FIG. 2K is a UMAP projection with increased resolution subdividing cluster 1 into two (clusters 2 and 5). FIGS. 2L-2Q show Violin plots showing expression of HE and endothelial type associated genes CD34 (FIG. 2L), NOTCH1 (FIG. 2M), DLL4 (FIG. 2N), EPHB4 (FIG. 2O), NR2F2 (FIG. 2P). FIG. 2Q shows immunofluorescence showing mCherry reporter accuracy by comparison with antibody staining of SOX17. Scale bars are 100 μm.

FIGS. 3A-3F show that SOX17 is the only SOXF member that increases HE progenitor differentiation efficiency when overexpressed. FIG. 3A is a schematic showing the generation of H9+XLone-SOXF (SOX7, SOX17, or SOX18) hPSCs. FIG. 3B shows flow cytometry analysis of SOX17 expression in H9+XLone-SOX17 cells treated with or without Dox for 24 hours. FIG. 3C is a schematic illustrating culture conditions for SOXF overexpression during HE progenitor differentiation. FIG. 3D shows immunofluorescence analysis of day 5 cells differentiated with Dox for each XLone-SOXF cell line and positive control without Dox. Cells were stained with SOX17 and VEC antibodies and nuclear stain (Hoechst 33342). Scale bars are 100 μm. FIG. 3E is a graph showing quantification of flow cytometry analysis of CD34, VEC, and CD31 expression in D5 H9+XLone-SOXF cells treated with Dox and control without Dox (n=3). ** and **** indicate p<0.01 and p<0.0001 respectively. Error bars represent standard error of the mean. FIG. 3F shows flow cytometry analysis of SOX17 expression in D5 H9+XLone_SOX17 cells differentiated with Dox.

FIGS. 4A-4C show day 5 cells resulting from differentiation with SOXF overexpression. FIG. 4A shows brightfield images of control, H9+XLone-SOX7, H9+XLone-SOX17, and H9+XLone-SOX18 cells differentiated to day 5 HE cells with and without Dox. Scale bars are 100 μm. FIG. 4B shows representative flow cytometry plots analyzing CD34, VEC, and CD31 expression in day 5 cells (n=3). FIG. 4C is a yield plot for day 5 cells. Error bars represent standard error of the mean. * indicates p<0.05.

FIGS. 5A-5F show that generation of hPSCs with inducible ETV2 overexpression and comparison of SOX17 and ETV2 forward programming mature endothelial cell potential. FIG. 5A is a schematic showing the generation of H9+XLone-ETV2 cells. FIG. 5B shows flow analysis showing ETV2 associated FLAG expression with and without 24 hours of Dox treatment. FIG. 5C shows immunofluorescence showing ubiquitous FLAG expression associated with ETV2. Scale bars are 100 μm. FIG. 5D shows qPCR data confirming an increase in ETV2 expression with Dox treatment (n=3). **** indicates p<0.0001. Error bar represents standard error of the mean. FIGS. 5E-F show immunofluorescence analysis of D5 SOX17 (FIG. 5E) and ETV2 (FIG. 5F) forward programmed cells that were further differentiated to D20 in commercially available endothelial cell differentiation media. Scale bars are 100 μm.

FIGS. 6A-6G show that SOX17 expression occurs immediately prior to endothelial marker expression and transcriptional interference reduces endothelial marker expression. FIG. 6A is an image of the Western blots showing SOX17 and VEC protein levels over the course of differentiation. β-actin was used as a housekeeping gene. FIG. 6B are graphs showing quantification of blots shown in (FIG. 6A) normalized to β-actin. FIG. 6C is a graph showing quantification of SOX17⁺VEC⁻ cells (left axis, orange) and SOX17⁺VEC⁺ cells (right axis, blue) in the SOX17⁺ population as derived from analysis of immunofluorescent images (n=10). Error bars represent standard error of the mean. FIG. 6D shows representative immunofluorescence images used for quantification in (FIG. 6C) for day 3.75 and day 5 (n=10). Orange arrows highlight SOX17⁺VEC⁻ cells and blue arrows highlight SOX17⁺VEC⁺ cells. Nuclear staining was done with Hoechst 33342. Scale bars are 100 μm. FIG. 6E is a schematic illustrating the generation of a cell line with Cas13d-based inducible SOX17 knockdown. FIG. 6F is a graph showing quantification of flow cytometry experiments analyzing the change in size of the day 5 population expressing CD31 or CD34 for cells treated with and without Dox (n=3). * indicates p<0.05 and **** indicates p<0.0001. Error bars represent standard error of the mean. FIG. 6G shows immunofluorescence images of D5 cells differentiated with or without Dox stained with VEC. Nuclear staining was done with Hoechst 33342. Scale bars are 100 μm.

FIGS. 7A-7D show that SOX17 expression occurs prior to VEC expression and repression prevents HE differentiation. FIG. 7A shows images showing full blots for western blots shown in FIG. 6. FIG. 7B shows representative immunofluorescent images of cells every six hours of the differentiation. Orange arrows indicate SOX17⁺ cells and blue arrows indicate SOX17⁺VEC⁺ cells. Scale bars are 100 μm. FIG. 7C shows flow analysis of D3 definitive endodermal cells differentiated with and without Dox treatment. FIG. 7D shows representative flow analysis plots showing CD31 and CD34 expression in D5 endothelial progenitors differentiated with and without Dox treatment (n=3).

FIG. 8 shows SOX17 and ETV2 forward programming. Daily brightfield images showing cells treated without Dox or with Dox for SOX17 and ETV2 overexpression cell lines. Scale bars are 130 μm.

FIGS. 9A-9J show that SOX17 forward programming is sufficient to produce CD34⁺VEC⁺ cells. FIG. 9A is a graph showing quantification of flow cytometry analysis of day 5 cells for endothelial progenitor markers VEC, CD34, and CD31. Day 5 SOX17 forward programmed cells are compared to ETV2 forward programmed cells. *** indicates p<0.001. FIG. 9B shows qPCR analysis of CD34 expression from day 0 to 5 for LaSR Basal+Dox condition. FIG. 9C shows immunofluorescence analysis of VEC expression in day 5 cells for each condition. Scale bars are 130 μm. FIG. 9D is a graph showing quantification of flow cytometry analysis for day 5 cells for endothelial progenitor markers CD34, VEC, and CD31 for cells treated with Dox for 3, 4, and 5 days. FIG. 9E shows representative flow cytometry plots showing SOX17 forward programmed cells with Dox treatment for 5 days. FIG. 9F is a graph showing quantification of flow cytometry analysis for different Dox concentrations used for 5 days of treatment. FIG. 9G is a graph showing quantification of day 5 flow cytometry data for day 5 cells cultured in LaSR hPSC media with or without Dox. **** indicates p<0.0001. FIG. 9H shows representative flow cytometry analysis of day 5 cells treated with or without Dox in LaSR hPSC media. FIG. 9I is a graph showing quantification of flow cytometry analysis of CD73 and VEC expression in day 5 cells. FIG. 9J shows representative flow cytometry plots for CD73 and VEC flow analysis.

FIGS. 10A-10F show H1+XLone-SOX17 forward programming. FIG. 10A is a schematic showing the generation of H1 OCT4-GFP+XLone-SOX17 cells. FIG. 10B shows flow analysis showing SOX17 expression with and without 24 hours of Dox treatment. FIG. 10C is an experimental schematic of forward programming protocol. FIGS. 10D-E show representative flow analysis of CD34, CD31, GFP (FIG. 10D), and VEC (FIG. 10E) expression for day 5 forward programmed cells (n=3). FIG. 10F is a graph showing a quantification of flow cytometry data (n=3). Error bars represent standard error of the mean.

FIGS. 11A-11C show SOX17 forward programming temporal and concentration optimization. FIG. 11A shows representative flow cytometry analysis for CD34, CD31, and VEC of SOX17 d5 forward programmed cells treated with Dox for 3 or 4 days (n=3). FIG. 11B shows representative flow cytometry analysis of CD34, CD31, and VEC for SOX17 d5 forward programmed cells treated with varied concentrations of Dox for 5 days (n=3). FIG. 11C shows flow cytometry analysis for CD34, CD31, and VEC of 6-9-9+XLone-SOX17 day 5 forward programmed cells treated with Dox for 5 days and passaged on day 2.

FIGS. 12A-12N show that SOX17 forward programming results in hematopoietic progenitors that significantly upregulate hematopoietic transcription factors and surface markers. FIG. 12A is a schematic of defined and growth factor free hematopoietic progenitor differentiation with forward programming duration indicated by Dox treatment. FIG. 12B shows flow cytometry analysis of CD34 expression and cell viability for day 8 floating cells. FIG. 12C is a heat map showing qPCR data for day 0, day 5, and day 11 suspension cells (n=3). Data is depicted as expression relative to day 0 and scaled by row. FIG. 12D shows flow cytometry analysis of CD34 expression and cell viability for day 8 floating cells. FIG. 12E shows the percentage of CD34+, CD45+, and CD44+ cells in day 10 hematopoietic progenitors. FIG. 12F is a heat map showing qPCR data for day 0, day 5, and day 11 suspension cells (n=3). Data is depicted as expression relative to day 0 and scaled by row. FIGS. 12G-N show quantification of gene expression determined by qPCR (n=3) for CD45 (FIG. 12G), CD43 (FIG. 12H), RUNX1 (FIG. 12I), SPI1 (FIG. 12J), ERG (FIG. 12K), HOXA5 (FIG. 12L), HOXA9 (FIG. 12M), and HOXA10 (FIG. 12N). *, **, ***, and **** indicates p<0.05, 0.01, 0.001, and 0.0001 respectively. Error bars represent standard error of the mean.

FIGS. 13A-13D show SOX17 forward programming in hPSC media. FIG. 13A shows bright field images of cells forward programmed with or without SOX17 overexpression in LaSR hPSC media. Scale bars are 100 μm. FIG. 13B shows bright field images of cells forward programmed with or without SOX17 overexpression in mTeSR1 hPSC media. Scale bars are 100 μm. FIG. 13C shows representative flow cytometry analysis of CD34 and VEC expression in day 5 cells (n=3). FIG. 13D) is a graph showing quantification of flow cytometry analysis for CD34, CD31, and VEC in day 5 cells (n=3). **** indicates p<0.0001. Error bars represent standard error of the mean.

FIGS. 14A-14B show combinatorial testing of SOX17 forward programming and HE differentiation factors for HE specification. Representative flow cytometry plots for day 5 cells differentiated with stepwise inclusion of HE specification factors without (FIG. 14A) or with (FIG. 14B) Dox treatment (n=3).

FIGS. 15A-15I show that SOX17 forward programming is dependent on 3-catenin. FIGS. 15A-C show quantification of flow analysis for endothelial progenitor markers VEC (FIG. 15A), CD31 (FIG. 15B), CD34 (FIG. 15C) with day 5 cells. FIG. 15D is a schematic showing the generation of H9 CTNNB1 KO+XLone-SOX17 hPSCs. FIG. 15E shows flow cytometry analysis validating the function of the H9 CTNNB1 KO+XLone-SOX17 cell line. FIG. 15F is a graph showing quantification of flow analysis for CD34, VEC, and CD31 for day 5 cells. FIG. 15G shows representative flow cytometry plots for H9 CTNNB1 KO+XLone-SOX17 day 5 forward programmed cells. **** indicates p<0.0001. Error bars represent standard error of the mean and n=3 for all data. FIGS. 15H-I show that inducible SOX17 hPSCs were used. Dox upregulated SOX17 expression (FIG. 15H) and CTNNB1 expression also upregulated (FIG. 15I) as a result of SOX17 overexpression.

FIGS. 16A-16C show comparative analysis of hematopoietic factors in differentiated and SOXF forward programmed cells. Quantification of gene expression determined by qPCR (n=3) for CD34 (FIG. 16A), VEC (FIG. 16A), CD45 (FIG. 16A), HOXA5 (FIG. 16B), HOXA9 (FIG. 16B), and HOXA10 (FIG. 16B), SPI1 (FIG. 16C), ERG (FIG. 16C), RUNX1 (FIG. 16C). * , **, ***, and **** indicates p<0.05, 0.01, 0.001, and 0.0001 respectively. Error bars represent standard error of the mean.

FIGS. 17A-17C show comparative analysis of hematopoietic factors in differentiated and SOXF forward programmed cells. Quantification of gene expression determined by qPCR (n=3) for CD34 (FIG. 17A), VEC (FIG. 17A), CD45 (FIG. 17A), HOXA5 (FIG. 17B), HOXA9 (FIG. 17B), and HOXA10 (FIG. 17B), SPI1 (FIG. 17C), ERG (FIG. 17C), RUNX1 (FIG. 17C). *, **, ***, and **** indicates p<0.05, 0.01, 0.001, and 0.0001 respectively. Error bars represent standard error of the mean.

FIGS. 18A-18C show that SOX17 forward programmed cells further differentiate to multiple hematopoietic lineages. FIG. 18A shows representative images of erythrocytic (E), granulocytic (G), macrophage (M), granulocytic/macrophage (GM), and granulocytic/erythrocytic/monocytic/megakaryocytic (GEMM) colony forming units. Scale bars are 200 μm. FIG. 18B is a graph showing quantification of colonies formed by day 8, 10, and 12 hematopoietic progenitors. FIG. 18C shows representative images of different hematopoietic cells obtained on day 10. Scale bars are 10 μm.

### DETAILED DESCRIPTION

A first aspect of the disclosure relates to a method of producing an enriched preparation of hemogenic endothelial progenitor cells. This method involves providing a population of pluripotent stem cells and inducing expression of a SOXF transcription factor in the pluripotent stem cells of the population. The method further involves culturing the population of pluripotent stem cells expressing the SOXF transcription factor, whereby the enriched preparation of hemogenic endothelial progenitor cells is produced as a result of said culturing.

Another aspect of the disclosure relates to a method of producing an enriched preparation of progenitor cells. This method involves providing a population of pluripotent stem cells and inducing expression of a SOXF transcription factor in the pluripotent stem cells of the population. The method further involves culturing the population of pluripotent stem cells expressing the SOXF transcription factor, whereby an enriched preparation of differentiated progenitor cells is produced as a result of said culturing.

In accordance with this and all aspects of the disclosure, the progenitor cells and hemogenic endothelial progenitor cells are derived from a population of pluripotent stem cells. In one embodiment, the pluripotent stem cells are a population of embryonic stem cells. Embryonic stem cells are derived from totipotent cells of the early mammalian embryo and are capable of unlimited, undifferentiated proliferation in vitro. Embryonic stem cells include cells isolated from an embryo, placenta, or umbilical cord, or immortalized versions of such cells, e.g., an embryonic stem cell line. In some embodiments, the population of embryonic stem cells is a population of human embryonic stem cells. In some embodiments, the population of embryonic stem cells is a human embryonic stem cell line. Suitable human embryonic stem cell lines include, without limitation, lines WA-01 (H1), WA-07 (H7), WA-09 (H9), WA-13 (H13), and WA-14 (H14) (Thomson et al., “Embryonic Stem Cell Lines Derived from Human Blastocytes,” Science 282 (5391): 1145-47 (1998) and U.S. Pat. No. 7,029,913 to Thomson et al., which are hereby incorporated by reference in their entirety). Other suitable embryonic stem cell lines includes the HAD-C100 cell line (Tannenbaum et al., “Derivation of Xeno-free and GMP-grade Human Embryonic Stem Cells—Platforms for Future Clinical Applications,” PLoS One 7(6):e35325 (2012), which is hereby incorporated by reference in its entirety), the WIBR4, WIBR5, WIBR6 cell lines (Lengner et al., “Derivation of Pre-x Inactivation Human Embryonic Stem Cell Line in Physiological Oxygen Conditions,” Cell 141(5):872-83 (2010), which is hereby incorporated by reference in its entirety), and the human embryonic stem cell lines (HUES) lines 1-17 (Cowan et al., “Derivation of Embryonic Stem-Cell Lines from Human Blastocytes,” N. Engl. J. Med. 350:1353-56 (2004), which is hereby incorporated by reference in its entirety).

In another embodiment, the population of pluripotent stem cells is a population of induced pluripotent stem cells (iPSC). Induced pluripotent stem cells are pluripotent cells that are derived from non-pluripotent cells, such as somatic cells or tissue stem cells, by inducing the expression of a combination of reprogramming factors in the non-pluripotent cells. Suitable reprogramming factors that promote and induce iPSC generation include one or more of Oct4, Klf4, Sox2, c-Myc, Nanog, C/EBPa, Esrrb, Lin28, and Nr5a2. In certain embodiments, at least two reprogramming factors are expressed in a somatic cell to successfully reprogram the somatic cell. In other embodiments, at least three reprogramming factors are expressed in a somatic cell to successfully reprogram the somatic cell. In other embodiments, at least four reprogramming factors are expressed in a somatic cell to successfully reprogram the somatic cell.

iPSCs suitable for use in the methods disclosed herein can be derived from any of a variety of non-pluripotent cells, including for example, adult fibroblasts (see e.g., Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi: 10.1093/eurheartj/ehs203 (2012), which is hereby incorporated by reference in its entirety), umbilical cord blood (see e.g., Cai et al., “Generation of Human Induced Pluripotent Stem Cells from Umbilical Cord Matrix and Amniotic Membrane Mesenchymal Cells,” J. Biol. Chem. 285(15): 112227-11234 (2110) and Giorgetti et al., “Generation of Induced Pluripotent Stem Cells from Human Cord Blood Cells with only Two Factors: Oct4 and Sox2,” Nature Protocols, 5(4):811-820 (2010), which are hereby incorporated by reference in their entirety), bone marrow (see e.g., Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi: 10.1093/eurheartj/ehs203 (Jul. 12, 2012), and Hu et al., “Efficient Generation of Transgene-Free Induced Pluripotent Stem Cells from Normal and Neoplastic Bone Marrow and Cord Blood Mononuclear Cells,” Blood doi: 10.1182/blood-2010-07-298331 (Feb. 4, 2011), which are hereby incorporated by reference in their entirety), and peripheral blood (see e.g., Sommer et al., “Generation of Human Induced Pluripotent Stem Cells from Peripheral Blood using the STEMCCA Lentiviral Vector,” J. Vis. Exp. 68: e4327 doi:10.3791/4327 (2012), which is hereby incorporated by reference in its entirety). iPSCs can also be derived from keratinocytes, mature B cells, mature T cells, pancreatic β cells, melanocytes, hepatocytes, foreskin cells, cheek cells, lung fibroblasts, myeloid progenitors, hematopoietic stem cells, adipose-derived stem cells, neural stem cells, and liver progenitor cells. In some embodiments, the iPSCs are human iPSCs.

iPSCs may be derived by methods known in the art including the use of integrating viral vectors (e.g., lentiviral vectors, inducible lentiviral vectors, and retroviral vectors), excisable vectors (e.g., transposon and floxed lentiviral vectors), and non-integrating vectors (e.g., adenoviral and plasmid vectors) to deliver genes encoding the aforementioned cell reprogramming factors (see e.g., Takahashi and Yamanaka, Cell 126:663-676 (2006); Okita. et al., Nature 448:313-317 (2007); Nakagawa et al., Nat. Biotechnol. 26:101-106 (2007); Takahashi et al., Cell 131:1-12 (2007); Meissner et al. Nat. Biotech. 25:1177-1181 (2007); Yu et al. Science 318:1917-1920 (2007); Park et al. Nature 451:141-146 (2008); and U.S. Patent Application Publication No. 2008/0233610, which are hereby incorporated by reference in their entirety). Other methods for generating iPSCs include those disclosed in WO2007/069666, WO2009/006930, WO2009/006997, WO2009/007852, WO2008/118820, U.S. Patent Application Publication Nos. 2011/0200568 to Ikeda et al., 2010/0156778 to Egusa et al., 2012/0276070 to Musick, and 2012/0276636 to Nakagawa, Shi et al., Cell Stem Cell 3(5): 568-574 (2008), Kim et al., Nature 454: 646-650 (2008), Kim et al., Cell 136(3):411-419 (2009), Huangfu et al., Nature Biotechnology 26: 1269-1275 (2008), Zhao et al., Cell Stem Cell 3: 475-479 (2008), Feng et al., Nature Cell Biology 11: 197-203 (2009), and Hanna et al., Cell 133(2): 250-264 (2008), which are hereby incorporated by reference in their entirety.

Integration free approaches, i.e., those using non-integrating and excisable vectors, for deriving iPSCs free of transgenic sequences are particularly suitable in the therapeutic context. Suitable methods of iPSC production that utilize non-integrating vectors include methods that use adenoviral vectors (Stadtfeld et al., “Induced Pluripotent Stem Cells Generated without Viral Integration,” Science 322: 945-949 (2008), and Okita et al., “Generation of Mouse Induced Pluripotent Stem Cells without Viral Vectors,” Science 322: 949-953 (2008), which are hereby incorporated by reference in their entirety), Sendi virus vectors (Fusaki et al., “Efficient Induction of Transgene-Free Human Pluripotent Stem Cells Using a Vector Based on Sendi Virus, an RNA Virus That Does Not Integrate into the Host Genome,” Proc Jpn Acad. 85: 348-362 (2009), which is hereby incorporated by reference in its entirety), polycistronic minicircle vectors (Jia et al., “A Nonviral Minicircle Vector for Deriving Hyman iPS Cells,” Nat. Methods 7: 197-199 (2010), which is hereby incorporated by reference in its entirety), and self-replicating selectable episomes (Yu et al., “Human Induced Pluripotent Stem Cells Free of Vector and Transgene Sequences,” Science 324: 797-801 (2009), which is hereby incorporated by reference in its entirety). Suitable methods for iPSC generation using excisable vectors are described by Kaji et al., “Virus-Free Induction of Pluripotency and Subsequent Excision of Reprogramming Factors,” Nature 458: 771-775 (2009), Soldner et al., “Parkinson's Disease Patient-Derived Induced Pluripotent Stem Cells Free of Viral Reprogramming Factors,” Cell 136:964-977 (2009), Woltjen et al., “PiggyBac Transposition Reprograms Fibroblasts to Induced Pluripotent Stem Cells,” Nature 458: 766-770 (2009), and Yusa et al., “Generation of Transgene-Free Induced Pluripotent Mouse Stem Cells by the PiggyBac Transposon,” Nat. Methods 6: 363-369 (2009), which are hereby incorporated by reference in their entirety. Suitable methods for iPSC generation also include methods involving the direct delivery of reprogramming factors as recombinant proteins (Zhou et al., “Generation of Induced Pluripotent Stem Cells Using Recombinant Proteins,” Cell Stem Cell 4: 381-384 (2009), which is hereby incorporated by reference in its entirety) or as whole-cell extracts isolated from ESCs (Cho et al., “Induction of Pluripotent Stem Cells from Adult Somatic Cells by Protein-Based Reprogramming without Genetic Manipulation,” Blood 116: 386-395 (2010), which is hereby incorporated by reference in its entirety).

In accordance with this aspect of the disclosure, the method of producing progenitor cells or hemogenic endothelial progenitor cells involves inducing the expression of a SOXF transcription factor in pluripotent stem cells. Suitable SOXF transcription factors include, without limitation, SOX-7, SOX-17, SOX-18, and any combination thereof.

Transcription factor SOX-17 is a transcriptional regulator that is heavily involved in various aspects of embryonic developmental. SOX-17 transcription factor is not normally expressed in pluripotent stem cells; however, as demonstrated herein, it was unexpectedly discovered that inducing expression of SOX17 in pluripotent stem cells is sufficient, alone, to drive differentiation of the pluripotent stem cells into cells of the hematopoietic lineage, including hemogenic endothelial cells and hematopoietic stem cells.

The transcription factor SOX-17 is encoded by the SOX17 gene. In some embodiments the method of inducing expression of SOX-17 transcription factor is achieved by introducing an expression vector comprising a nucleotide sequence encoding the transcription factor SOX-17 into pluripotent stem cells. In some embodiments, the nucleotide sequence encodes human SOX-17 or a fragment thereof. In some embodiments, the nucleotide sequence encodes human SOX-17 comprising an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 2 (shown below). In some embodiments, the nucleotide sequence encoding the SOX-17 transcription factor is the human SOX17 genomic sequence. In some embodiments, the nucleotide sequence encoding SOX-17 transcription factor is the human SOX17 mRNA sequence comprising a nucleotide sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 1 (GenBank Accession No. AB073988).

| (SEQ ID NO: 1) | |
|---|---|
| 1 | gccatgagca gcccggatgc gggatacgcc agtgacgacc agagccagac ccagagcgcg |
| 61 | ctgcccgcgg tgatggccgg gctgggcccc tgcccctggg ccgagtcgct gagccccatc |
| 121 | ggggacatga aggtgaaggg cgaggcgccg gcgaacagcg gagcaccggc cggggccgcg |
| 181 | ggccgagcca agggcgagtc ccgtatccgg cggccgatga acgctttcat ggtgtgggct |
| 241 | aaggacgagc gcaagcggct ggcgcagcag aatccagacc tgcacaacgc cgagttgagc |
| 301 | aagatgctgg gcaagtcgtg gaaggcgctg acgctggcgg agaagcggcc cttcgtggag |
| 361 | gaggcagagc ggctgcgcgt gcagcacatg caggaccacc ccaactacaa gtaccggccg |
| 421 | cggcggcgca agcaggtgaa gcggctgaag cgggtggagg gcggcttcct gcacggcctg |
| 481 | gctgagccgc aggcggccgc gctgggcccc gagggcggcc gcgtggccat ggacggcctg |
| 541 | ggcctccagt tccccgagca gggcttcccc gccggcccgc cgctgctgcc tccgcacatg |
| 601 | ggcggccact accgcgactg ccagagtctg ggcgcgcctc cgctcgacgg ctacccgttg |
| 661 | cccacgcccg acacgtcccc gctggacggc gtggaccccg acccggcttt cttcgccgcc |
| 721 | ccgatgcccg gggactgccc ggcggccggc acctacagct acgcgcaggt ctcggactac |
| 781 | gctggccccc cggagcctcc cgccggtccc atgcaccccc gactcggccc agagcccgcg |
| 841 | ggtccctcga ttccgggcct cctggcgcca cccagcgccc ttcacgtgta ctacggcgcg |
| 901 | atgggctcgc ccggggcggg cggcgggcgc ggcttccaga tgcagccgca acaccagcac |
| 961 | cagcaccagc accagcacca ccccccgggc cccggacagc cgtcgccccc tccggaggca |
| 1021 | ctgccctgcc gggacggcac ggaccccagt cagcccgccg agctcctcgg ggaggtggac |
| 1081 | cgcacggaat ttgaacagta tctgcacttc gtgtgcaagc ctgagatggg cctcccctac |
| 1141 | caggggcatg actccggtgt gaatctcccc gacagccacg gggccatttc ctcggtggtg |
| 1201 | tccgacgcca gctccgcggt atattactgc aactatcctg acgtgtgaca ggtccctgat |
| 1261 | ccgccccagc ctgcaggcca gaagcagtgt tacacacttc ctggaggagc taaggaaatc |
| 1321 | ctcagactcc tgggtttttg ttgttgctgt tgttgttttt taaaaggtgt gttggcatat |
| 1381 | aatttatggt aatttatttt gtctgccact tgaacagttt gggggggtga ggtttcattt |
| 1441 | aaaatttgtt cagagatttg tttcccatag ttggattgtc aaaaccctat ttccaagttc |
| 1501 | aagttaacta gctttgaatg tgtcccaaaa cagcttcctc catttcctga aagtttattg |
| 1561 | atcaaagaaa tgttgtcctg ggtgtgtttt ttcaatcttc taaaaaataa aatctggaat |
| 1621 | cctgcttttt tgctctacta gtacctctgt cacacta |

In some embodiments the method of inducing expression of SOX-17 transcription factor is achieved by introducing an expression vector comprising a nucleotide sequence of SEQ ID NO: 1 or a fragment thereof.

The protein encoded by the SOX17 mRNA nucleotide sequence comprises an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 2 (GenBank Accession No. BAB83867) as provided below or a functional fragment thereof.

| (SEQ ID NO: 2) | |
|---|---|
| 1 | msspdagyas ddqsqtqsal pavmaglgpc pwaeslspig dmkvkgeapa nsgapagaag |
| 61 | rakgesrirr pmnafmvwak derkrlaqqn pdlhnaelsk mlgkswkalt laekrpfvee |
| 121 | aerlrvqhmq dhpnykyrpr rrkqvkrlkr veggflhgla epqaaalgpe ggrvamdglg |
| 181 | lqfpeqgfpa gppllpphmg ghyrdcqslg appldgyplp tpdtspldgv dpdpaffaap |
| 241 | mpgdcpaagt ysyaqvsdya gppeppagpm hprlgpepag psipgllapp salhvyygam |
| 301 | gspgagggrg gqmqpqhqhq hqhqhhppgp gqpspppeal pcrdgtdpsq paellgevdr |
| 361 | tefeqylhfv ckpemglpyq hgdsgvnlpd shgaissvvs dassavyycn ypdv |

In some embodiments, the nucleotide sequence encoding the transcription factor SOX-17 is a modified RNA sequence. In some embodiments, the modified SOX-17 RNA sequence comprises a nucleotide sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 3, which contains the coding region of SOX-17 flanked by the untranslated regions of the beta globin gene.

ACANNNGCNNCNGACACAACNGNGNNCACNAGCAACCNCAAACAGACACC
GGANCCANGGCANGCGAANNCGCNAGCAAGCNNGGNACCGCCACCANGAG
CAGCCCGGANGCGGGANACGCCAGNGACGACCAGAGCCAGACCCAGAGCG
CGCNGCCCGCGGNGANGGCCGGGCNGGGCCCCNGCCCCNGGGCCGAGNCG
CNGAGCCCCANCGGGGACANGAAGGNGAAGGGCGAGGCGCCGGCGAACAG
CGGAGCACCGGCCGGGGCCGCGGGCCGAGCCAAGGGCGAGNCCCGNANCC
GGCGGCCGANGAACGCNNNCANGGNGNGGGCNAAGGACGAGCGCAAGCGG
CNGGCGCAGCAGAANCCAGACCNGCACAACGCCGAGNNGAGCAAGANGCN
GGGCAAGNCGNGGAAGGCGCNGACGCNGGCGGAGAAGCGGCCCNNCGNGG
AGGAGGCAGAGCGGCNGCGCGNGCAGCACANGCAGGACCACCCCAACNAC
AAGNACCGGCCGCGGCGGCGCAAGCAGGNGAAGCGGCNGAAGCGGGNGGA
GGGCGGCNNCCNGCACGGCCNGGCNGAGCCGCAGGCGGCCGCGCNGGGCC
CCGAGGGCGGCCGCGNGGCCANGGACGGCCNGGGCCNCCAGNNCCCCGAG
CAGGGCNNCCCCGCCGGCCCGCCGCNGCNGCCNCCGCACANGGGCGGCCA
CNACCGCGACNGCCAGAGNCNGGGCGCGCCNCCGCNCGACGGCNACCCGN
NGCCCACGCCCGACACGNCCCCGCNGGACGGCGNGGACCCCGACCCGGCN
NNCNNCGCCGCCCCGANGCCCGGGGACNGCCCGGCGGCCGGCACCNACAG
CNACGCGCAGGNCNCGGACNACGCNGGCCCCCCGGAGCCNCCCGCCGGNC
CCANGCACCCCCGACNCGGCCCAGAGCCCGCGGGNCCCNCGANNCCGGGC
CNCCNGGCGCCACCCAGCGCCCNNCACGNGNACNACGGCGCGANGGGCNC
GCCCGGGGCGGGCGGCGGGCGCGGCNNCCAGANGCAGCCGCAACACCAGC
ACCAGCACCAGCACCAGCACCACCCCCCGGGCCCCGGACAGCCGNCGCCC
CCNCCGGAGGCACNGCCCNGCCGGGACGGCACGGACCCCAGNCAGCCCGC
CGAGCNCCNCGGGGAGGNGGACCGCACGGAANNNGAACAGNANCNGCACN
NCGNGNGCAAGCCNGAGANGGGCCNCCCCNACCAGGGGCANGACNCCGGN
GNGAANCNCCCCGACAGCCACGGGGCCANNNCCNCGGNGGNGNCCGACGC
CAGCNCCGCGGNANANNACNGCAACNANCCNGACGNGNAGACNAGNGAGC
NCGCNNNCNNGCNGNCCAANNNCNANNAAAGGNNCCNNNGNNCCCNAAGN
CCAACNACNAAACNGGGGGANANNANGAAGGGCCNNGAGCANCNGGANNC
NGCCNAANAAAAAACANNNANNNNCANNGCAA

In some embodiments, the modified SOX-17 RNA sequence comprises a nucleotide sequence of SEQ ID NO: 3. In some embodiments, the nucleotide sequence of SEQ ID NO: 3 further comprises a poly A tail comprising about 120 adenosine residues.

In some embodiments, N in the RNA sequence of SEQ ID NO: 3 comprises uracil. In some embodiments, the RNA sequence of SEQ ID NO: 3 contains one or more modified nucleobases that reduce immunogenicity of the RNA. In some embodiments, N in the RNA sequence of SEQ ID NO: 3 is uracil, a modified uracil selected from the group consisting of pseudouridine, N1-methylpseudouridine, 5-methoxyuridine, 5-methyluridine, 2-thiouridine, and any combination thereof, or any combination or uracil and modified uracil bases. In some embodiments, N in SEQ ID NO: 3 is pseudouridine.

Another aspect of the present disclosure relates to a recombinant nucleic acid construct comprising the nucleotide sequence of SEQ ID NO: 3 (SOX17) or a nucleotide sequence having at least 85% sequence identity to the nucleotide sequence of SEQ ID NO: 3.

In some embodiments the SOXF transcription factor is a SOX-7 transcription factor. The transcription factor SOX-7 is encoded by the SOX7 gene. In some embodiments the method of inducing expression of SOX-7 transcription factor is achieved by introducing an expression vector comprising a nucleotide sequence encoding the transcription factor SOX-7 into pluripotent stem cells. In some embodiments, the nucleotide sequence encodes human SOX-7 or a fragment thereof. In some embodiments, the nucleotide sequence encodes human SOX-7 comprising an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 11 (shown below). In some embodiments, the nucleotide sequence encoding the SOX-7 transcription factor is the human SOX-7 genomic sequence. In some embodiments, the nucleotide sequence encoding SOX-7 transcription factor is the human SOX-7 mRNA sequence comprising a nucleotide sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 4 (NCBI Ref Seq. NM_031439.4).

(SEQ ID NO: 4)
Atggcttcgctgctgggagcctacccttggcccgagggtctcgagtgccc
ggccctggacgccgagctgtcggatggacaatcgccgccggccgtccccc
ggcccccgggggacaagggctccgagagccgtatccggcggcccatgaac
gccttcatggtttgggccaaggacgagaggaaacggctggcagtgcagaa
cccggacctgcacaacgccgagctcagcaagatgctgggaaagtcgtgga
aggcgctgacgctgtcccagaagaggccgtacgtggacgaggcggagcgg
ctgcgcctgcagcacatgcaggactaccccaactacaagtaccggccgcg
caggaagaagcaggccaagcggctgtgcaagcgcgtggacccgggcttcc
ttctgagctccctctcccgggaccagaacgccctgccggagaagagaagc
ggcagccggggggcgctgggggagaaggaggacaggggtgagtactcccc
cggcactgccctgcccagcctccggggctgctaccacgaggggccggctg
gtggtggcggcggcggcaccccgagcagtgtggacacgtacccgtacggg
ctgcccacacctcctgaaatgtctcccctggacgtgctggagccggagca
gaccttcttctcctccccctgccaggaggagcatggccatccccgccgca
tcccccacctgccagggcacccgtactcaccggagtacgccccaagccct
ctccactgtagccaccccctgggctccctggcccttggccagtcccccgg
cgtctccatgatgtcccctgtacccggctgtcccccatctcctgcctatt
actccccggccacctaccacccactccactccaacctccaagcccacctg
ggccagctttccccgcctcctgagcaccctggcttcgacgccctggatca
actgagccaggtggaactcctgggggacatggatcgcaatgaattcgacc
agtatttgaacactcctggccacccagactccgccacaggggccatggcc
ctcagtgggcatgttccggtctcccaggtgacaccaacgggtcccacaga
gaccagcctcatctccgtcctggctgatgccacggccacgtactacaaca
gctacagtgtgtcatag

The protein encoded by the SOX-7 mRNA nucleotide sequence comprises an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 11 (NCBI Ref Seq. No. NP_113627.1) as provided below or a functional fragment thereof.

| | |
|---|---|
| 1 | masllgaypw peglecpald aelsdgqspp avprppgdkg sesrirrpmn afmvwakder |
| 61 | krlavqnpdl hnaelskmlg kswkaltlsq krpyvdeaer lrlqhmqdyp nykyrprrkk |
| 121 | qakrlckrvd pgfllsslsr dqnalpekrs gsrgalgeke drgeyspgta lpslrgcyhe |
| 181 | gpaggggggt pssvdtypyg lptppemspl dvlepeqtff sspcqeehgh prriphlpgh |
| 241 | pyspeyapsp lhcshplgsl algqspgvsm mspvpgcpps payyspatyh plhsnlqahl |
| 301 | gqlspppehp gfdaldqlsq vellgdmdrn efdqylntpg hpdsatgama lsghvpvsqv |
| 361 | tptgptetsl isvladatat yynsysvs |

In some embodiments, the nucleotide sequence encoding the transcription factor SOX-7 is a modified RNA sequence. In some embodiments, the modified RNA sequence encoding SOX-7 contains one or more modified nucleobases that reduce immunogenicity of the RNA. In some embodiments, the uracils of the SOX-7 RNA (shown as thymine in SEQ ID NO: 4) are each substituted with a modified residue independently selected from pseudouridine, N1-methylpseudouridine, 5-methoxyuridine, 5-methyluridine, and 2-thiouridine.

In some embodiments the SOXF transcription factor is a SOX-18 transcription factor. The transcription factor SOX-18 is encoded by the SOX18 gene. In some embodiments the method of inducing expression of SOX-18 transcription factor is achieved by introducing an expression vector comprising a nucleotide sequence encoding the transcription factor SOX-18 into pluripotent stem cells. In some embodiments, the nucleotide sequence encodes human SOX-18 or a fragment thereof. In some embodiments, the nucleotide sequence encodes human SOX-18 comprising an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 12 (shown below). In some embodiments, the nucleotide sequence encoding the SOX-18 transcription factor is the human SOX18 genomic sequence. In some embodiments, the nucleotide sequence encoding SOX-18 transcription factor is the human SOX18 mRNA sequence comprising a nucleotide sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 1 SEQ ID NO: 5 (NCBI Ref Seq NM_018419.3).

(SEQ ID NO: 5)
Atgcagagatcgccgcccggctacggcgcacaggacgacccgcccgcccg
ccgcgactgtgcatgggccccgggacacggggccgccgctgacacgcgcg
gcctcgccgccggccccgccgccctcgccgcgcccgccgcgcccgcctcg
ccgcccagcccgcagcgcagtcccccgcgcagccccgagccggggcgcta
tggcctcagcccggccggccgcggggaacgccaggcggcagacgagtcgc
gcatccggcggcccatgaacgccttcatggtgtgggcaaaggacgagcgc
aagcggctggctcagcagaacccggacctgcacaacgcggtgctcagcaa
gatgctgggcaaagcgtggaaggagctgaacgcggcggagaagcggccct
tcgtggaggaagccgaacggctgcgcgtgcagcacttgcgcgaccacccc
aactacaagtaccggccgcgccgcaagaagcaggcgcgcaaggcccggcg
gctggagcccggcctcctgctcccgggattagcgcccccgcagccaccgc
ccgagcctttccccgcggcgtctggctcggctcgcgccttccgcgagctg
cccccgctgggcgccgagttcgacggcctggggctgcccacgcccgagcg
ctcgcctctggacggcctggagcccggcgaggctgccttcttcccaccgc
ccgcggcgcccgaggactgcgcgctgcggcccttccgcgcgccctacgcg
cccaccgagttgtcgcgggaccccggcggttgctacggggctcccctggc
ggaggcgctcaggaccgcgccccccgcggcgccgctcgctggcctgtact
acggcaccctgggcacgcccggcccgtaccccggcccgctgtcgccgccg
cccgaggccccgccgctggagagcgccgagccgctggggcccgccgccga
tctgtgggccgacgtggacctcaccgagttcgaccagtacctcaactgca
gccggactcggcccgacgcccccgggctcccgtaccacgtggcactggcc
aaactgggcccgcgcgccatgtcctgcccagaggagagcagcctgatctc
cgcgctgtcggacgccagcagcgcggtctattacagcgcgtgcatctccg
gctag

The protein encoded by the SOX-18 mRNA nucleotide sequence comprises an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 12 (NCBI Ref. Seq. NP_060889.1) as provided below or a functional fragment thereof.

| | |
|---|---|
| 1 | mqrsppgyga qddpparrdc awapghgaaa dtrglaagpa alaapaapas ppspqrsppr |
| 61 | spepgrygls pagrgerqaa desrirrpmn afmvwakder krlaqqnpdl hnavlskmlg |
| 121 | kawkelnaae krpfveeaer 1rvqhlrdhp nykyrprrkk qarkarrlep glllpglapp |
| 181 | qpppepfpaa sgsarafrel pplgaefdgl glptperspl dglepgeaaf fpppaapedc |
| 241 | alrpfrapya ptelsrdpgg cygaplaeal rtappaapla glyygtlgtp gpypgplspp |
| 301 | peapplesae plgpaadlwa dvdltefdqy lncsrtrpda pglpyhvala klgpramscp |
| 361 | eesslisals dassavyysa cisg |

In some embodiments, the nucleotide sequence encoding the transcription factor SOX-18 is a modified RNA sequence. In some embodiments, the modified RNA sequence encoding SOX-18 contains one or more modified nucleobases that reduce immunogenicity of the RNA. In some embodiments, the uracil of the SOX-7 mRNA nucleotide sequence (shown as thymine in SEQ ID NO: 5 above) is substituted with a modified residue selected from pseudouridine, N1-methylpseudouridine, 5-methoxyuridine, 5-methyluridine, and 2-thiouridine.

In some embodiments, the nucleic acid molecule introduced in cells of the pluripotent stem cell population to induce expression of a SOXF transcription factor encodes a mouse SOXF transcription factor, e.g., a mouse SOX-7, SOX-17, SOX-18 or combination thereof. The nucleotide and amino acid sequences of the mouse transcription factor SOX-17 are known in the art, see, e.g., UniProt Accession No. Q61473. The nucleotide and amino acid sequences of the mouse transcription factor SOX-7 are known in the art, see e.g., UniProt Accession No. P40646. The nucleotide and amino acid sequences of the mouse transcription factor SOX-18 are known in the art, see, e.g., UniProt Accession No. P43680.

In some embodiments the nucleotide sequence encoding the SOXF transcription factor encodes a rat SOXF transcription factor, e.g., a rat SOX-7, SOX-17, SOX-18 or combination thereof. The nucleotide and amino acid sequences of the rat transcription factor SOX-17 are known in the art, see, e.g., UniProt Accession No. G3V923. The nucleotide and amino acid sequences of the rat transcription factor SOX-7 are known in the art, see, e.g., UniProt Accession No. D3ZTE1. The nucleotide and amino acid sequences of the rat transcription factor SOX-18 are known in the art, see, e.g., UniProt Accession No. Q4V7E4.

In some embodiments, the nucleotide sequence encoding the SOXF transcription factor encodes a chimpanzee SOXF transcription factor, e.g., a chimpanzee SOX-7, SOX-17, SOX-18 or combination thereof. The nucleotide and amino acid sequences of the chimpanzee transcription factor SOX-17 are known in the art, see, e.g., UniProt Accession No. H2QW62. The nucleotide and amino acid sequences of the chimpanzee transcription factor SOX-7 are known in the art, see, e.g., UniProt Accession No. A0A2I3S8Z2. The nucleotide and amino acid sequences of the chimpanzee transcription factor SOX-18 are known in the art, see, e.g., UniProt Accession No. H2QKU0.

In some embodiments, the nucleotide sequence encoding the SOXF transcription factor encodes a canine SOXF transcription factor, e.g., a dog SOX-7, SOX-17, SOX-18 or combination thereof. The nucleotide and amino acid sequences of canine transcription factor SOX-17 are known in the art, see, e.g., UniProt Accession No. J9NWY6. The nucleotide and amino acid sequences of the canine transcription factor SOX-7 are known in the art, see, e.g., UniProt Accession No. F1P113. The nucleotide and amino acid sequences of the canine transcription factor SOX-18 are known in the art, see, e.g., UniProt Accession No. J9NSH5.

In some embodiments, the nucleotide sequence encoding the SOXF transcription factor encodes a bovine SOXF transcription factor, e.g., a bovine SOX-7, SOX-17, SOX-18 or combination thereof. The nucleotide and amino acid sequences of the bovine transcription factor SOX-17 are known in the art, see, e.g., UniProt Accession No. F1N1F9. The nucleotide and amino acid sequences of the bovine transcription factor SOX-7 are known in the art, see, e.g., UniProt Accession No. A0A3Q1LUX0. The nucleotide and amino acid sequences of the bovine transcription factor SOX-18 are known in the art, see, e.g., UniProt Accession No. Q0VC26.

In some embodiments, the nucleotide sequence encoding the SOXF transcription factor encodes a pig SOXF transcription factor, e.g., a pig SOX-7, SOX-17, SOX-18 or combination thereof. The nucleotide and amino acid sequences of the pig transcription factor SOX-17 are known in the art, see, e.g., UniProt Accession No. F1RSI1. The nucleotide and amino acid sequences of the pig transcription factor SOX-7 are known in the art, see, e.g., UniProt Accession No. A0A287A8T2. The nucleotide and amino acid sequences of the pig transcription factor SOX-18 are known in the art, see, e.g., UniProt Accession No. A0A287APF5.

Other transcription factor SOX-7, SOX-17, and SOX-18 sequences readily known in the art can also be utilized in the methods described herein.

In accordance with the methods disclosed herein, suitable nucleotide sequences encoding the SOXF transcription factors include, for example, known genomic or mRNA sequences encoding human SOX-7, SOX-17, and SOX-18 transcription factor, e.g., the nucleotide sequences of SEQ ID NO: 1, 4, and 5, as well as variants thereof encoding functional SOXF transcription factors. In particular, as a result of degeneracy of the genetic code, nucleotide sequences having about 80%, 85%, 90%, 95%, 96%, 97%, 98%, or 99% sequence identity to the known SOXF transcription factor genomic or mRNA sequence are also suitable for use in the methods disclosed herein. In particular, nucleotide sequences having about 80%, 85%, 90%, 95%, 96%, 97%, 98%, or 99% sequence identity to the nucleotide sequence of SEQ ID NO: 1 are suitable for use in the methods disclosed herein. Likewise, nucleotide sequences having about 80%, 85%, 90%, 95%, 96%, 97%, 98%, or 99% sequence identity to the nucleotide sequences of SEQ ID NO: 4 and SEQ ID NO: 5 are suitable for use in the methods disclosed herein.

In some embodiments, the SOXF transcription factor is induced by introducing the SOXF transcription factor nucleic acid molecule, e.g., SOXF transcription factor RNA, directly into the cells. Alternatively, in some embodiments, the SOXF transcription factor expression is induced by introducing an expression vector comprising a nucleotide sequence (mRNA or genomic sequence) encoding the SOXF transcription factor into the population of pluripotent stem cells. Suitable expression vectors are known in the art and include, without limitation, integrating viral vectors (e.g., lentiviral vectors, inducible lentiviral vectors, and retroviral vectors), excisable vectors (e.g., transposon and floxed lentiviral vectors), and non-integrating vectors (e.g., adenoviral and plasmid vectors).

In some embodiments, the expression vector is a plasmid vector (see, e.g., Muthumani et al., “Optimized and Enhanced DNA Plasmid Vector Based In vivo Construction of a Neutralizing anti-HIV-1 Envelope Glycoprotein Fab,” Hum. Vaccin. Immunother. 9: 2253-2262 (2013), which is hereby incorporated by reference in its entirety). Plasmids can transform a target cell by integration into the cellular genome or exist extrachromosomally (e.g., autonomous replicating plasmid with an origin of replication). Exemplary plasmid vectors include, without limitation, pCEP4, pREP4, pVAX, pcDNA3.0, provax, or any other expression vector commonly used in the art to effectuate mammalian gene expression.

In some embodiments, the expression vector is a linear expression cassette (“LEC”). LECs are capable of being efficiently delivered to cells via electroporation to express the SOXF transcription factor protein encoded by the SOXF transcription factor nucleotide sequence. The LEC may be any linear DNA devoid of a phosphate backbone. In some embodiments, the LEC does not contain any antibiotic resistance genes and/or a phosphate backbone. The LEC may be derived from any plasmid capable of being linearized and expressing the SOXF transcription factor encoded by the SOXF transcription factor nucleotide sequence. Exemplary plasmids include, without limitation, pNP (Puerto Rico/34), pM2 (New Caledonia/99), WLV009, pVAX, pcDNA3.0, or provax.

In some embodiments, the expression vector is a viral vector. Suitable viral vectors that are capable of expressing full length proteins include, for example and without limitation, adeno-associated virus (AAV) vectors, lentivirus vectors, retrovirus vectors, replication deficient adenovirus vectors, and gutless adenovirus vectors. Methods for generating adeno-associated viruses (AAVs) suitable for use as expression vectors are known in the art (see, e.g., Grieger & Samulski, “Adeno-associated Virus as a Gene Therapy Vector: Vector Development, Production and Clinical Applications,” Adv. Biochem. Engin Biotechnol. 99: 119-145 (2005); Buning et al, “Recent Developments in Adeno-associated Virus Vector Technology,” J. Gene Med. 10:717-733 (2008), each of which is incorporated herein by reference in its entirety).

In some embodiments, the expression vector utilized to induce SOXF transcription factor expression in pluripotent stem cells according to the methods of the present disclosure is the XLone plasmid containing a Tet-On drug inducible system as described by Randolph et al., “An All-in-One, Tet-On 3G Inducible PiggyBac System for Human Pluripotent Stem Cells and Derivatives,” Scientific Reports 7:1549 (2017), which is hereby incorporated by reference in its entirety.

In some embodiments, the nucleic acid molecule encoding SOX17 suitable for use in accordance with the methods described herein is contained in the XLone plasmid. This construct comprises the nucleotide sequence of SEQ ID NO: 6 as shown below.

(SEQ ID NO: 6)
CAGGAACGCGAGCTGATTTTCCAGGGTTTCGTACTGTTTCTCTGTTGGGCGGGTGCCGAGATGCACTTTAGCCCCGTCGCGATGTGAGA
GGAGAGCACAGCGGTATGACTTGGCGTTGTTCCGCAGAAAGTCTTGCCATGACTCGCCTTCCAGGGGGCAGGAGTGGGTATGATGCCTG
TCCAGCATCTCGATTGGCAGGGCATCGAGCAGGGCCCGCTTGTTCTTCACGTGCCAGTACAGGGTAGGCTGCTCAACTCCCAGCTTTTG
AGCGAGTTTCCTTGTCGTCAGGCCTTCGATACCGACTCCATTGAGTAATTCCAGAGCAGAGTTTATGACTTTGCTCTTGTCCAGTCTAG
ACATCTTATCGTCATCGTCTTTGTAATCCATGGTGGCGGATCCCGCGTCACGACACCTGTGTTCTGGCGGCAAACCCGTTGCGAAAAAG
AACGTTCACGGCGACTACTGCACTTATATACGGTTCTCCCCCACCCTCGGGAAAAAGGCGGAGCCAGTACACGACATCACTTTCCCAGT
TTACCCCGCGCCACCTTCTCTAGGCACCGGTTCAATTGCCGACCCCTCCCCCCAACTTCTCGGGGACTGTGGGCGATGTGCGCTCTGCC
CACTGACGGGCACCGGAGCCACTCGAGTGGAATTATCACCTCGAGTTTACTCCCTATCAGTGATAGAGAACGTATGAAGAGTTTACTCC
CTATCAGTGATAGAGAACGTATGCAGACTTTACTCCCTATCAGTGATAGAGAACGTATAAGGAGTTTACTCCCTATCAGTGATAGAGAA
CGTATGACCAGTTTACTCCCTATCAGTGATAGAGAACGTATCTACAGTTTACTCCCTATCAGTGATAGAGAACGTATATCCAGTTTACT
CCCTATCAGTGATAGAGAACGTATAAGCTTTGCTTATGTAAACCAGGGCGCCTATAAAAGAGTGCTGATTTTTTGAGTAAACTTCAATT
CCACAACACTTTTGTCTTATACCAACTTTCCGTACCACTTCCTACCCTCGTAAAGGTACCGCCACCATGAGCAGCCCGGATGCGGGATA
CGCCAGTGACGACCAGAGCCAGACCCAGAGCGCGCTGCCCGCGGTGATGGCCGGGCTGGGCCCCTGCCCCTGGGCCGAGTCGCTGAGCC
CCATCGGGGACATGAAGGTGAAGGGCGAGGCGCCGGCGAACAGCGGAGCACCGGCCGGGGCCGCGGGCCGAGCCAAGGGCGAGTCCCGT
ATCCGGCGGCCGATGAACGCTTTCATGGTGTGGGCTAAGGACGAGCGCAAGCGGCTGGCGCAGCAGAATCCAGACCTGCACAACGCCGA
GTTGAGCAAGATGCTGGGCAAGTCGTGGAAGGCGCTGACGCTGGCGGAGAAGCGGCCCTTCGTGGAGGAGGCAGAGCGGCTGCGCGTGC
AGCACATGCAGGACCACCCCAACTACAAGTACCGGCCGCGGCGGCGCAAGCAGGTGAAGCGGCTGAAGCGGGTGGAGGGCGGCTTCCTG
CACGGCCTGGCTGAGCCGCAGGCGGCCGCGCTGGGCCCCGAGGGCGGCCGCGTGGCCATGGACGGCCTGGGCCTCCAGTTCCCCGAGCA
GGGCTTCCCCGCCGGCCCGCCGCTGCTGCCTCCGCACATGGGCGGCCACTACCGCGACTGCCAGAGTCTGGGCGCGCCTCCGCTCGACG
GCTACCCGTTGCCCACGCCCGACACGTCCCCGCTGGACGGCGTGGACCCCGACCCGGCTTTCTTCGCCGCCCCGATGCCCGGGGACTGC
CCGGCGGCCGGCACCTACAGCTACGCGCAGGTCTCGGACTACGCTGGCCCCCCGGAGCCTCCCGCCGGTCCCATGCACCCCCGACTCGG
CCCAGAGCCCGCGGGTCCCTCGATTCCGGGCCTCCTGGCGCCACCCAGCGCCCTTCACGTGTACTACGGCGCGATGGGCTCGCCCGGGG
CGGGCGGCGGGCGCGGCTTCCAGATGCAGCCGCAACACCAGCACCAGCACCAGCACCAGCACCACCCCCCGGGCCCCGGACAGCCGTCG
CCCCCTCCGGAGGCACTGCCCTGCCGGGACGGCACGGACCCCAGTCAGCCCGCCGAGCTCCTCGGGGAGGTGGACCGCACGGAATTTGA
ACAGTATCTGCACTTCGTGTGCAAGCCTGAGATGGGCCTCCCCTACCAGGGGCATGACTCCGGTGTGAATCTCCCCGACAGCCACGGGG
CCATTTCCTCGGTGGTGTCCGACGCCAGCTCCGCGGTATATTACTGCAACTATCCTGACGTGTAGACTAGTAGACCACCTCCCCTGCGA
GCTAAGCTGGACAGCCAATGACGGGTAAGAGAGTGACATTTTTCACTAACCTAAGACAGGAGGGCCGTCAGAGCTACTGCCTAATCCAA
AGACGGGTAAAAGTGATAAAAATGTATCACTCCAACCTAAGACAGGCGCAGCTTCCGAGGGATTTGAGATCCAGACATGATAAGATACA
TTGATGAGTTTGGACAAACCAAAACTAGAATGCAGTGAAAAAAATGCCTTATTTGTGAAATTTGTGATGCTATTGCCTTATTTGTAACC
ATTATAAGCTGCAATAAACAAGTTTGATATCTATAACAAGAAAATATATATATAATAAGTTATCACGTAAGTAGAACATGAAATAACAA
TATAATTATCGTATGAGTTAAATCTTAAAAGTCACGTAAAAGATAATCATGCGTCATTTTGACTCACGCGGTCGTTATAGTTCAAAATC
AGTGACACTTACCGCATTGACAAGCACGCCTCACGGGAGCTCCAAGCGGCGACTGAGATGTCCTAAATGCACAGCGACGGATTCGCGCT
ATTTAGAAAGAGAGAGCAATATTTCAAGAATGCATGCGTCAATTTTACGCAGACTATCTTTCTAGGGTTAAGAATTCACTGGCCGTCGT
TTTACAACGTCGTGACTGGGAAAACCCTGGCGTTACCCAACTTAATCGCCTTGCAGCACATCCCCCTTTCGCCAGCTGGCGTAATAGCG
AAGAGGCCCGCACCGATCGCCCTTCCCAACAGTTGCGCAGCCTGAATGGCGAATGGCGCCTGATGCGGTATTTTCTCCTTACGCATCTG
TGCGGTATTTCACACCGCATATGGTGCACTCTCAGTACAATCTGCTCTGATGCCGCATAGTTAAGCCAGCCCCGACACCCGCCAACACC
CGCTGACGCGCCCTGACGGGCTTGTCTGCTCCCGGCATCCGCTTACAGACAAGCTGTGACCGTCTCCGGGAGCTGCATGTGTCAGAGGT
TTTCACCGTCATCACCGAAACGCGCGAGACGAAAGGGCCTCGTGATACGCCTATTTTTATAGGTTAATGTCATGATAATAATGGTTTCT
TAGACGTCAGGTGGCACTTTTCGGGGAAATGTGCGCGGAACCCCTATTTGTTTATTTTTCTAAATACATTCAAATATGTATCCGCTCAT
GAGACAATAACCCTGATAAATGCTTCAATAATATTGAAAAAGGAAGAGTATGAGTATTCAACATTTCCGTGTCGCCCTTATTCCCTTTT
TTGCGGCATTTTGCCTTCCTGTTTTTGCTCACCCAGAAACGCTGGTGAAAGTAAAAGATGCTGAAGATCAGTTGGGTGCACGAGTGGGT
TACATCGAACTGGATCTCAACAGCGGTAAGATCCTTGAGAGTTTTCGCCCCGAAGAACGTTTTCCAATGATGAGCACTTTTAAAGTTCT
GCTATGTGGCGCGGTATTATCCCGTATTGACGCCGGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTATGCAGTGCTGCCATAACCATGAGTGATAACACTGCG
GCCAACTTACTTCTGACAACGATCGGAGGACCGAAGGAGCTAACCGCTTTTTTGCACAACATGGGGGATCATGTAACTCGCCTTGATCG
TTGGGAACCGGAGCTGAATGAAGCCATACCAAACGACGAGCGTGACACCACGATGCCTGTAGCAATGGCAACAACGTTGCGCAAACTAT
TAACTGGCGAACTACTTACTCTAGCTTCCCGGCAACAATTAATAGACTGGATGGAGGCGGATAAAGTTGCAGGACCACTTCTGCGCTCG
GCCCTTCCGGCTGGCTGGTTTATTGCTGATAAATCTGGAGCCGGTGAGCGTGGGTCTCGCGGTATCATTGCAGCACTGGGGCCAGATGG
TAAGCCCTCCCGTATCGTAGTTATCTACACGACGGGGAGTCAGGCAACTATGGATGAACGAAATAGACAGATCGCTGAGATAGGTGCCT
CACTGATTAAGCATTGGTAACTGTCAGACCAAGTTTACTCATATATACTTTAGATTGATTTAAAACTTCATTTTTAATTTAAAAGGATC
TAGGTGAAGATCCTTTTTGATAATCTCATGACCAAAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGAGCGCAGATACCAAATACTGTTCTTCTAGTGTAGCCGTAG
TTAGGCCACCACTTCAAGAACTCTGTAGCACCGCCTACATACCTCGCTCTGCTAATCCTGTTACCAGTGGCTGCTGCCAGTGGCGATAA
GTCGTGTCTTACCGGGTTGGACTCAAGACGATAGTTACCGGATAAGGCGCAGCGGTCGGGCTGAACGGGGGGTTCGTGCACACAGCCCA
GCTTGGAGCGAACGACCTACACCGAACTGAGATACCTACAGCGTGAGCTATGAGAAAGCGCCACGCTTCCCGAAGGGAGAAAGGCGGAC
AGGTATCCGGTAAGCGGCAGGGTCGGAACAGGAGAGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCTGATTCTGTGGATAACCGTATTACCGCCTTT
GAGTGAGCTGATACCGCTCGCCGCAGCCGAACGACCGAGCGCAGCGAGTCAGTGAGCGAGGAAGCGGAAGAGCGCCCAATACGCAAACC
GCCTCTCCCCGCGCGTTGGCCGATTCATTAATGCAGCTGGCACGACAGGTTTCCCGACTGGAAAGCGGGCAGTGAGCGCAACGCAATTA
ATGTGAGTTAGCTCACTCATTAGGCACCCCAGGCTTTACACTTTATGCTTCCGGCTCGTATGTTGTGTGGAATTGTGAGCGGATAACAA
TTTCACACAGGAAACAGCTATGACCATGATTACGCCAAGGTCGACTTAACCCTAGAAAGATAATCATATTGTGACGTACGTTAAAGATA
ATCATGCGTAAAATTGACGCATGTGTTTTATCGGTCTGTATATCGAGGTTTATTTATTAATTTGAATAGATATTAAGTTTTATTATATT
TACACTTACATACTAATAATAAATTCAACAAACAATTTATTTATGTTTATTTATTTATTAAAAAAAAACAAAAACTCAAAATTTCTTCT
ATAAAGTAACAAAACTTTTAGCAGTGAAAAAAATGCTTTATTTGTGAAATTTGTGATGCTATTGCTTTATTTGTAACCATTATAAGCTG
CAATAAACAAGTTAACAACAACAATTGCATTCATTTTATGTTTCAGGTTCAGGGGGAGGTGTGGGAGGTTTTTTAAAGCAAGTAAAACC
TCTACAAATGTGGTATGGCTGATTATGATCCTCTGGAGATTTAGCCCTCCCACACATAACCAGAGGGCAGCAATTCACGAATCCCAACT
GCCGTCGGCTGTCCATCACTGTCCTTCACTATGGCTTTGATCCCAGGATGCAGATCGAGAAGCACCTGTCGGCACCGTCCGCAGGGGCT
CAAGATGCCCCTGTTCTCATTTCCGATCGCGACGATACAAGTCAGGTTGCCAGCTGCCGCAGCAGCAGCAGTGCCCAGCACCACGAGTT
CTGCACAAGGTCCCCCAGTAAAATGATATACATTGACACCAGTGAAGATGCGGCCGTCGCTAGAGAGAGCTGCGCTGGCGACGCTGTAG
TCTTCAGAGATGGGGATGCTGTTGATTGTAGCCGTTGCTCTTTCAATGAGGGTGGATTCTTCTTGAGACAAAGGCTTGGCCATGGGGCC
GGGGTTCTCCTCCACGTCGCCGGCCTGCTTCAGCAGGCTGAAGTTGGTGGCGCCGCTGCCCCCGGGGAGCATGTCAAGGTCAAAATCGT
CAAGAGCGTCAGCAGGCAGCATATCAAGGTCAAAGTCGTCAAGGGCATCGGCTGGGAGCATGTCTAAGTCAAAATCGTCAAGGGCGTCG
GTCGGCCCGCCGCTTTCGCACTTTAGCTGTTTCTCCAGGCCACATATGATTAGTTCCAGGCCGAAAAGGAAGGCAGGTTCGGCTCCCTG
CCGGTCGAACAGCTCAATTGCTTGTTTCAGAAGTGGGGGCATAGAATCGGTGGTAGGTGTCTCTCTTTCCTCTTTTGCTACTTGATGCT
CCTGTTCCTCCAATACGCAGCCCAGTGTAAAGTGGCCCACGGCGGACAGAGCGTACAGTGCGTTCTCCAGGGAGAAGCCTTGCTGACA

In some embodiments, the nucleic acid molecule encoding SOX7 suitable for use in accordance with the methods described herein is contained in the XLone plasmid. This construct comprises the nucleotide sequence of SEQ ID NO: 7 as shown below.

(SEQ ID NO: 7)
GAGTTTACTCCCTATCAGTGATAGAGAACGTATGAAGAGTTTACTCCCTATCAGTGATAGAGAACGTATGCAGACTTTACTCCCT
ATCAGTGATAGAGAACGTATAAGGAGTTTACTCCCTATCAGTGATAGAGAACGTATGACCAGTTTACTCCCTATCAGTGATAGAG
AACGTATCTACAGTTTACTCCCTATCAGTGATAGAGAACGTATATCCAGTTTACTCCCTATCAGTGATAGAGAACGTATAAGCTT
TGCTTATGTAAACCAGGGCGCCTATAAAAGAGTGCTGATTTTTTGAGTAAACTTCAATTCCACAACACTTTTGTCTTATACCAAC
TTTCCGTACCACTTCCTACCCTCGTAAAGGTACCGCCACCATGGCTTCGCTGCTGGGAGCCTACCCTTGGCCCGAGGGTCTCGAG
TGCCCGGCCCTGGACGCCGAGCTGTCGGATGGACAATCGCCGCCGGCCGTCCCCCGGCCCCCGGGGGACAAGGGCTCCGAGAGCC
GTATCCGGCGGCCCATGAACGCCTTCATGGTTTGGGCCAAGGACGAGAGGAAACGGCTGGCAGTGCAGAACCCGGACCTGCACAA
CGCCGAGCTCAGCAAGATGCTGGGAAAGTCGTGGAAGGCGCTGACGCTGTCCCAGAAGAGGCCGTACGTGGACGAGGCGGAGCGG
CTGCGCCTGCAGCACATGCAGGACTACCCCAACTACAAGTACCGGCCGCGCAGGAAGAAGCAGGCCAAGCGGCTGTGCAAGCGCG
TGGACCCGGGCTTCCTTCTGAGCTCCCTCTCCCGGGACCAGAACGCCCTGCCGGAGAAGAGAAGCGGCAGCCGGGGGGCGCTGGG
GGAGAAGGAGGACAGGGGTGAGTACTCCCCCGGCACTGCCCTGCCCAGCCTCCGGGGCTGCTACCACGAGGGGCCGGCTGGTGGT
GGCGGCGGCGGCACCCCGAGCAGTGTGGACACGTACCCGTACGGGCTGCCCACACCTCCTGAAATGTCTCCCCTGGACGTGCTGG
AGCCGGAGCAGACCTTCTTCTCCTCCCCCTGCCAGGAGGAGCATGGCCATCCCCGCCGCATCCCCCACCTGCCAGGGCACCCGTA
CTCACCGGAGTACGCCCCAAGCCCTCTCCACTGTAGCCACCCCCTGGGCTCCCTGGCCCTTGGCCAGTCCCCCGGCGTCTCCATG
ATGTCCCCTGTACCCGGCTGTCCCCCATCTCCTGCCTATTACTCCCCGGCCACCTACCACCCACTCCACTCCAACCTCCAAGCCC
ACCTGGGCCAGCTTTCCCCGCCTCCTGAGCACCCTGGCTTCGACGCCCTGGATCAACTGAGCCAGGTGGAACTCCTGGGGGACAT
GGATCGCAATGAATTCGACCAGTATTTGAACACTCCTGGCCACCCAGACTCCGCCACAGGGGCCATGGCCCTCAGTGGGCATGTT
CCGGTCTCCCAGGTGACACCAACGGGTCCCACAGAGACCAGCCTCATCTCCGTCCTGGCTGATGCCACGGCCACGTACTACAACA
GCTACAGTGTGTCATAGACTAGTAGACCACCTCCCCTGCGAGCTAAGCTGGACAGCCAATGACGGGTAAGAGAGTGACATTTTTC
ACTAACCTAAGACAGGAGGGCCGTCAGAGCTACTGCCTAATCCAAAGACGGGTAAAAGTGATAAAAATGTATCACTCCAACCTAA
GACAGGCGCAGCTTCCGAGGGATTTGAGATCCAGACATGATAAGATACATTGATGAGTTTGGACAAACCAAAACTAGAATGCAGT
GAAAAAAATGCCTTATTTGTGAAATTTGTGATGCTATTGCCTTATTTGTAACCATTATAAGCTGCAATAAACAAGTTTGATATCT
ATAACAAGAAAATATATATATAATAAGTTATCACGTAAGTAGAACATGAAATAACAATATAATTATCGTATGAGTTAAATCTTAA
AAGTCACGTAAAAGATAATCATGCGTCATTTTGACTCACGCGGTCGTTATAGTTCAAAATCAGTGACACTTACCGCATTGACAAG
CACGCCTCACGGGAGCTCCAAGCGGCGACTGAGATGTCCTAAATGCACAGCGACGGATTCGCGCTATTTAGAAAGAGAGAGCAAT
ATTTCAAGAATGCATGCGTCAATTTTACGCAGACTATCTTTCTAGGGTTAAGAATTCACTGGCCGTCGTTTTACAACGTCGTGAC
TGGGAAAACCCTGGCGTTACCCAACTTAATCGCCTTGCAGCACATCCCCCTTTCGCCAGCTGGCGTAATAGCGAAGAGGCCCGCA
CCGATCGCCCTTCCCAACAGTTGCGCAGCCTGAATGGCGAATGGCGCCTGATGCGGTATTTTCTCCTTACGCATCTGTGCGGTAT
TTCACACCGCATATGGTGCACTCTCAGTACAATCTGCTCTGATGCCGCATAGTTAAGCCAGCCCCGACACCCGCCAACACCCGCT
GACGCGCCCTGACGGGCTTGTCTGCTCCCGGCATCCGCTTACAGACAAGCTGTGACCGTCTCCGGGAGCTGCATGTGTCAGAGGT
TTTCACCGTCATCACCGAAACGCGCGAGACGAAAGGGCCTCGTGATACGCCTATTTTTATAGGTTAATGTCATGATAATAATGGT
TTCTTAGACGTCAGGTGGCACTTTTCGGGGAAATGTGCGCGGAACCCCTATTTGTTTATTTTTCTAAATACATTCAAATATGTAT
CCGCTCATGAGACAATAACCCTGATAAATGCTTCAATAATATTGAAAAAGGAAGAGTATGAGTATTCAACATTTCCGTGTCGCCC
TTATTCCCTTTTTTGCGGCATTTTGCCTTCCTGTTTTTGCTCACCCAGAAACGCTGGTGAAAGTAAAAGATGCTGAAGATCAGTT
GGGTGCACGAGTGGGTTACATCGAACTGGATCTCAACAGCGGTAAGATCCTTGAGAGTTTTCGCCCCGAAGAACGTTTTCCAATG
ATGAGCACTTTTAAAGTTCTGCTATGTGGCGCGGTATTATCCCGTATTGACGCCGGGCAAGAGCAACTCGGTCGCCGCATACACT
ATTCTCAGAATGACTTGGTTGAGTACTCACCAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGAGGACCGAAGGAGCTAACCGCTTTTTTGCAC
AACATGGGGGATCATGTAACTCGCCTTGATCGTTGGGAACCGGAGCTGAATGAAGCCATACCAAACGACGAGCGTGACACCACGA
TGCCTGTAGCAATGGCAACAACGTTGCGCAAACTATTAACTGGCGAACTACTTACTCTAGCTTCCCGGCAACAATTAATAGACTG
GATGGAGGCGGATAAAGTTGCAGGACCACTTCTGCGCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCCCTCCCGTATCGTAGTTATCTACACGACGGGGAGTC
AGGCAACTATGGATGAACGAAATAGACAGATCGCTGAGATAGGTGCCTCACTGATTAAGCATTGGTAACTGTCAGACCAAGTTTA
CTCATATATACTTTAGATTGATTTAAAACTTCATTTTTAATTTAAAAGGATCTAGGTGAAGATCCTTTTTGATAATCTCATGACC
AAAATCCCTTAACGTGAGTTTTCGTTCCACTGAGCGTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTGGTTTGTTTGCCGGATCAAGAGCTACCAACTCTTTT
TCCGAAGGTAACTGGCTTCAGCAGAGCGCAGATACCAAATACTGTTCTTCTAGTGTAGCCGTAGTTAGGCCACCACTTCAAGAAC
TCTGTAGCACCGCCTACATACCTCGCTCTGCTAATCCTGTTACCAGTGGCTGCTGCCAGTGGCGATAAGTCGTGTCTTACCGGGT
TGGACTCAAGACGATAGTTACCGGATAAGGCGCAGCGGTCGGGCTGAACGGGGGGTTCGTGCACACAGCCCAGCTTGGAGCGAAC
GACCTACACCGAACTGAGATACCTACAGCGTGAGCTATGAGAAAGCGCCACGCTTCCCGAAGGGAGAAAGGCGGACAGGTATCCG
GTAAGCGGCAGGGTCGGAACAGGAGAGCGCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTATGGAAAAACGCCAGCAACGCGGCCTTTTT
ACGGTTCCTGGCCTTTTGCTGGCCTTTGCTCACATGTTCTTTCCTGCGTTATCCCCTGATTCTGTGGATAACCGTATTACCGCCT
TTTGAGTGAGCTGATACCGCTCGCCGCAGCCGAACGACCGAGCGCAGCGAGTCAGTGAGCGAGGAAGCGGAAGAGCGCCCAATAC
GCAAACCGCCTCTCCCCGCGCGTTGGCCGATTCATTAATGCAGCTGGCACGACAGGTTTCCCGACTGGAAAGCGGGCAGTGAGCG
CAACGCAATTAATGTGAGTTAGCTCACTCATTAGGCACCCCAGGCTTTACACTTTATGCTTCCGGCTCGTATGTTGTGTGGAATT
GTGAGCGGATAACAATTTCACACAGGAAACAGCTATGACCATGATTACGCCAAGGTCGACTTAACCCTAGAAAGATAATCATATT
GTGACGTACGTTAAAGATAATCATGCGTAAAATTGACGCATGTGTTTTATCGGTCTGTATATCGAGGTTTATTTATTAATTTGAA
TAGATATTAAGTTTTATTATATTTACATACATACTAATAATAAATTCAACAAACAATTTATTTATGTTTATTTATTTATTAAAAA
AAAAAACAAAAACTCAAAATTTCTTCTATAAAGTAACAAAACTTTTAGCAGTGAAAAAAATGCTTTATTTGTGAAATTTGTGATG
CTATTGCTTTATTTGTAACCATTATAAGCTGCAATAAACAAGTTAACAACAACAATTGCATTCATTTTATGTTTCAGGTTCAGGG
GGAGGTGTGGGAGGTTTTTTAAAGCAAGTAAAACCTCTACAAATGTGGTATGGCTGATTATGATCCTCTGGAGATTTAGCCCTCC
CACACATAACCAGAGGGCAGCAATTCACGAATCCCAACTGCCGTCGGCTGTCCATCACTGTCCTTCACTATGGCTTTGATCCCAG
GATGCAGATCGAGAAGCACCTGTCGGCACCGTCCGCAGGGGCTCAAGATGCCCCTGTTCTCATTTCCGATCGCGACGATACAAGT
CAGGTTGCCAGCTGCCGCAGCAGCAGCAGTGCCCAGCACCACGAGTTCTGCACAAGGTCCCCCAGTAAAATGATATACATTGACA
CCAGTGAAGATGCGGCCGTCGCTAGAGAGAGCTGCGCTGGCGACGCTGTAGTCTTCAGAGATGGGGATGCTGTTGATTGTAGCCG
TTGCTCTTTCAATGAGGGTGGATTCTTCTTGAGACAAAGGCTTGGCCATGGGGCCGGGGTTCTCCTCCACGTCGCCGGCCTGCTT
CAGCAGGCTGAAGTTGGTGGCGCCGCTGCCCCCGGGGAGCATGTCAAGGTCAAAATCGTCAAGAGCGTCAGCAGGCAGCATATCA
AGGTCAAAGTCGTCAAGGGCATCGGCTGGGAGCATGTCTAAGTCAAAATCGTCAAGGGCGTCGGTCGGCCCGCCGCTTTCGCACT
TTAGCTGTTTCTCCAGGCCACATATGATTAGTTCCAGGCCGAAAAGGAAGGCAGGTTCGGCTCCCTGCCGGTCGAACAGCTCAAT
TGCTTGTTTCAGAAGTGGGGGCATAGAATCGGTGGTAGGTGTCTCTCTTTCCTCTTTTGCTACTTGATGCTCCTGTTCCTCCAAT
ACGCAGCCCAGTGTAAAGTGGCCCACGGCGGACAGAGCGTACAGTGCGTTCTCCAGGGAGAAGCCTTGCTGACACAGGAACGCGA
GCTGATTTTCCAGGGTTTCGTACTGTTTCTCTGTTGGGCGGGTGCCGAGATGCACTTTAGCCCCGTCGCGATGTGAGAGGAGAGC
ACAGCGGTATGACTTGGCGTTGTTCCGCAGAAAGTCTTGCCATGACTCGCCTTCCAGGGGGCAGGAGTGGGTATGATGCCTGTCC
AGCATCTCGATTGGCAGGGCATCGAGCAGGGCCCGCTTGTTCTTCACGTGCCAGTACAGGGTAGGCTGCTCAACTCCCAGCTTTT
GAGCGAGTTTCCTTGTCGTCAGGCCTTCGATACCGACTCCATTGAGTAATTCCAGAGCAGAGTTTATGACTTTGCTCTTGTCCAG
TCTAGACATCTTATCGTCATCGTCTTTGTAATCCATGGTGGCGGATCCCGCGTCACGACACCTGTGTTCTGGCGGCAAACCCGTT
GCGAAAAAGAACGTTCACGGCGACTACTGCACTTATATACGGTTCTCCCCCACCCTCGGGAAAAAGGCGGAGCCAGTACACGACA
TCACTTTCCCAGTTTACCCCGCGCCACCTTCTCTAGGCACCGGTTCAATTGCCGACCCCTCCCCCCAACTTCTCGGGGACTGTGG
GCGATGTGCGCTCTGCCCACTGACGGGCACCGGAGCCACTCGAGTGGAATTATCACCTC

In some embodiments, the nucleic acid molecule encoding SOX18 suitable for use in accordance with the methods described herein is contained in the XLone plasmid. This construct comprises the nucleotide sequence of SEQ ID NO: 8 as shown below.

(SEQ ID NO: 8)
TCCCTATCAGTGATAGAGAACGTATGAAGAGTTTACTCCCTATCAGTGATAGAGAACGTATGCAGACTTTACTCCCTATCAGT
GATAGAGAACGTATAAGGAGTTTACTCCCTATCAGTGATAGAGAACGTATGACCAGTTTACTCCCTATCAGTGATAGAGAACG
TATCTACAGTTTACTCCCTATCAGTGATAGAGAACGTATATCCAGTTTACTCCCTATCAGTGATAGAGAACGTATAAGCTTTG
CTTATGTAAACCAGGGCGCCTATAAAAGAGTGCTGATTTTTTGAGTAAACTTCAATTCCACAACACTTTTGTCTTATACCAAC
TTTCCGTACCACTTCCTACCCTCGTAAAGGTACCGCCACCATGCAGAGATCGCCGCCCGGCTACGGCGCACAGGACGACCCGC
CCGCCCGCCGCGACTGTGCATGGGCCCCGGGACACGGGGCCGCCGCTGACACGCGCGGCCTCGCCGCCGGCCCCGCCGCCCTC
GCCGCGCCCGCCGCGCCCGCCTCGCCGCCCAGCCCGCAGCGCAGTCCCCCGCGCAGCCCCGAGCCGGGGCGCTATGGCCTCAG
CCCGGCCGGCCGCGGGGAACGCCAGGCGGCAGACGAGTCGCGCATCCGGCGGCCCATGAACGCCTTCATGGTGTGGGCAAAGG
ACGAGCGCAAGCGGCTGGCTCAGCAGAACCCGGACCTGCACAACGCGGTGCTCAGCAAGATGCTGGGCAAAGCGTGGAAGGAG
CTGAACGCGGCGGAGAAGCGGCCCTTCGTGGAGGAAGCCGAACGGCTGCGCGTGCAGCACTTGCGCGACCACCCCAACTACAA
GTACCGGCCGCGCCGCAAGAAGCAGGCGCGCAAGGCCCGGCGGCTGGAGCCCGGCCTCCTGCTCCCGGGATTAGCGCCCCCGC
AGCCACCGCCCGAGCCTTTCCCCGCGGCGTCTGGCTCGGCTCGCGCCTTCCGCGAGCTGCCCCCGCTGGGCGCCGAGTTCGAC
GGCCTGGGGCTGCCCACGCCCGAGCGCGCCTCTGGACGGCCTGGAGCCCGGCGAGGCTGCCTTCTTCCCACCGCCCGCGGCGC
GCCCGAGGACTGCGCGCTGCGGCCCTTCCGCGCGCCCTACGCGCCCACCGAGTTGTCGCGGGACCCCGGCGGTTGCTACGGGG
CTCCCCTGGCGGAGGCGCTCAGGACCGCGCCCCCCGCGGCGCCGCTCGCTGGCCTGTACTACGGCACCCTGGGCACGCCCGGC
CCGTACCCCGGCCCGCTGTCGCCGCCGCCCGAGGCCCCGCCGCTGGAGAGCGCCGAGCCGCTGGGGCCCGCCGCCGATCTGTG
GGCCGACGTGGACCTCACCGAGTTCGACCAGTACCTCAACTGCAGCCGGACTCGGCCCGACGCCCCCGGGCTCCCGTACCACG
TGGCACTGGCCAAACTGGGCCCGCGCGCCATGTCCTGCCCAGAGGAGAGCAGCCTGATCTCCGCGCTGTCGGACGCCAGCAGC
GCGGTCTATTACAGCGCGTGCATCTCCGGCTAGACTAGTAGACCACCTCCCCTGCGAGCTAAGCTGGACAGCCAATGACGGGT
AAGAGAGTGACATTTTTCACTAACCTAAGACAGGAGGGCCGTCAGAGCTACTGCCTAATCCAAAGACGGGTAAAAGTGATAAA
AATGTATCACTCCAACCTAAGACAGGCGCAGCTTCCGAGGGATTTGAGATCCAGACATGATAAGATACATTGATGAGTTTGGA
CAAACCAAAACTAGAATGCAGTGAAAAAAATGCCTTATTTGTGAAATTTGTGATGCTATTGCCTTATTTGTAACCATTATAAG
CTGCAATAAACAAGTTTGATATCTATAACAAGAAAATATATATATAATAAGTTATCACGTAAGTAGAACATGAAATAACAATA
TAATTATCGTATGAGTTAAATCTTAAAAGTCACGTAAAAGATAATCATGCGTCATTTTGACTCACGCGGTCGTTATAGTTCAA
AATCAGTGACACTTACCGCATTGACAAGCACGCCTCACGGGAGCTCCAAGCGGCGACTGAGATGTCCTAAATGCACAGCGACG
GATTCGCGCTATTTAGAAAGAGAGAGCAATATTTCAAGAATGCATGCGTCAATTTTACGCAGACTATCTTTCTAGGGTTAAGA
ATTCACTGGCCGTCGTTTTACAACGTCGTGACTGGGAAAACCCTGGCGTTACCCAACTTAATCGCCTTGCAGCACATCCCCCT
TTCGCCAGCTGGCGTAATAGCGAAGAGGCCCGCACCGATCGCCCTTCCCAACAGTTGCGCAGCCTGAATGGCGAATGGCGCCT
GATGCGGTATTTTCTCCTTACGCATCTGTGCGGTATTTCACACCGCATATGGTGCACTCTCAGTACAATCTGCTCTGATGCCG
CATAGTTAAGCCAGCCCCGACACCCGCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGAAACGCGCGAGACGAAAGGGCCTCGT
GATACGCCTATTTTTATAGGTTAATGTCATGATAATAATGGTTTCTTAGACGTCAGGTGGCACTTTTCGGGGAAATGTGCGCG
GAACCCCTATTTGTTTATTTTTCTAAATACATTCAAATATGTATCCGCTCATGAGACAATAACCCTGATAAATGCTTCAATAA
TATTGAAAAAGGAAGAGTATGAGTATTCAACATTTCCGTGTCGCCCTTATTCCCTTTTTTGCGGCATTTTGCCTTCCTGTTTT
TGCTCACCCAGAAACGCTGGTGAAAGTAAAAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAGCACTTTTAAAGTTCTGCTATGTGGCGCG
GTATTATCCCGTATTGACGCCGGGCAAGAGCAACTCGGTCGCCGCATACACTATTCTCAGAATGACTTGGTTGAGTACTCACC
AGTCACAGAAAAGCATCTTACGGATGGCATGACAGTAAGAGAATTATGCAGTGCTGCCATAACCATGAGTGATAACACTGCGG
CCAACTTACTTCTGACAACGATCGGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACTGGATGGAGGCGGATAAAGTTGCAG
GACCACTTCTGCGCTCGGCCCTTCCGGCTGGCTGGTTTATTGCTGATAAATCTGGAGCCGGTGAGCGTGGGTCTCGCGGTATC
ATTGCAGCACTGGGGCCAGATGGTAAGCCCTCCCGTATCGTAGTTATCTACACGACGGGGAGTCAGGCAACTATGGATGAACG
AAATAGACAGATCGCTGAGATAGGTGCCTCACTGATTAAGCATTGGTAACTGTCAGACCAAGTTTACTCATATATACTTTAGA
TTGATTTAAAACTTCATTTTTAATTTAAAAGGATCTAGGTGAAGATCCTTTTTGATAATCTCATGACCAAAATCCCTTAACGT
GAGTTTTCGTTCCACTGAGCGTCAGACCCCGTAGAAAAGATCAAAGGATCTTCTTGAGATCCTTTTTTTCTGCGCGTAATCTG
CTGCTTGCAAACAAAAAAACCACCGCTACCAGCGGTGGTTTGTTTGCCGGATCAAGAGCTACCAACTCTTTTTCCGAAGGTAA
CTGGCTTCAGCAGAGCGCAGATACCAAATACTGTTCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCACACAGCCCAGCTTGGAGCGAACGACCT
ACACCGAACTGAGATACCTACAGCGTGAGCTATGAGAAAGCGCCACGCTTCCCGAAGGGAGAAAGGCGGACAGGTATCCGGTA
AGCGGCAGGGTCGGAACAGGAGAGCGCACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAGTCAGTGAGCGAGGAAGCGGAAGAGCGCCC
AATACGCAAACCGCCTCTCCCCGCGCGTTGGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTTACACTTTATGCTTCCGGCTCGTATGTTG
TGTGGAATTGTGAGCGGATAACAATTTCACACAGGAAACAGCTATGACCATGATTACGCCAAGGTCGACTTAACCCTAGAAAG
ATAATCATATTGTGACGTACGTTAAAGATAATCATGCGTAAAATTGACGCATGTGTTTTATCGGTCTGTATATCGAGGTTTAT
TTATTAATTTGAATAGATATTAAGTTTTATTATATTTACACTTACATACTAATAATAAATTCAACAAACAATTTATTTATGTT
TATTTATTTATTAAAAAAAAACAAAAACTCAAAATTTCTTCTATAAAGTAACAAAACTTTTAGCAGTGAAAAAAATGCTTTAT
TTGTGAAATTTGTGATGCTATTGCTTTATTTGTAACCATTATAAGCTGCAATAAACAAGTTAACAACAACAATTGCATTCATT
TTATGTTTCAGGTTCAGGGGGAGGTGTGGGAGGTTTTTTAAAGCAAGTAAAACCTCTACAAATGTGGTATGGCTGATTATGAT
CCTCTGGAGATTTAGCCCTCCCACACATAACCAGAGGGCAGCAATTCACGAATCCCAACTGCCGTCGGCTGTCCATCACTGTC
CTTCACTATGGCTTTGATCCCAGGATGCAGATCGAGAAGCACCTGTCGGCACCGTCCGCAGGGGCTCAAGATGCCCCTGTTCT
CATTTCCGATCGCGACGATACAAGTCAGGTTGCCAGCTGCCGCAGCAGCAGCAGTGCCCAGCACCACGAGTTCTGCACAAGGT
CCCCCAGTAAAATGATATACATTGACACCAGTGAAGATGCGGCCGTCGCTAGAGAGAGCTGCGCTGGCGACGCTGTAGTCTTC
AGAGATGGGGATGCTGTTGATTGTAGCCGTTGCTCTTTCAATGAGGGTGGATTCTTCTTGAGACAAAGGCTTGGCCATGGGGC
CGGGGTTCTCCTCCACGTCGCCGGCCTGCTTCAGCAGGCTGAAGTTGGTGGCGCCGCTGCCCCCGGGGAGCATGTCAAGGTCA
AAATCGTCAAGAGCGTCAGCAGGCAGCATATCAAGGTCAAAGTCGTCAAGGGCATCGGCTGGGAGCATGTCTAAGTCAAAATC
GTCAAGGGCGTCGGTCGGCCCGCCGCTTTCGCACTTTAGCTGTTTCTCCAGGCCACATATGATTAGTTCCAGGCCGAAAAGGA
AGGCAGGTTCGGCTCCCTGCCGGTCGAACAGCTCAATTGCTTGTTTCAGAAGTGGGGGCATAGAATCGGTGGTAGGTGTCTCT
CTTTCCTCTTTTGCTACTTGATGCTCCTGTTCCTCCAATACGCAGCCCAGTGTAAAGTGGCCCACGGCGGACAGAGCGTACAG
TGCGTTCTCCAGGGAGAAGCCTTGCTGACACAGGAACGCGAGCTGATTTTCCAGGGTTTCGTACTGTTTCTCTGTTGGGCGGG
TGCCGAGATGCACTTTAGCCCCGTCGCGATGTGAGAGGAGAGCACAGCGGTATGACTTGGCGTTGTTCCGCAGAAAGTCTTGC
CATGACTCGCCTTCCAGGGGGCAGGAGTGGGTATGATGCCTGTCCAGCATCTCGATTGGCAGGGCATCGAGCAGGGCCCGCTT
GTTCTTCACGTGCCAGTACAGGGTAGGCTGCTCAACTCCCAGCTTTTGAGCGAGTTTCCTTGTCGTCAGGCCTTCGATACCGA
CTCCATTGAGTAATTCCAGAGCAGAGTTTATGACTTTGCTCTTGTCCAGTCTAGACATCTTATCGTCATCGTCTTTGTAATCC
ATGGTGGCGGATCCCGCGTCACGACACCTGTGTTCTGGCGGCAAACCCGTTGCGAAAAAGAACGTTCACGGCGACTACTGCAC
TTATATACGGTTCTCCCCCACCCTCGGGAAAAAGGCGGAGCCAGTACACGACATCACTTTCCCAGTTTACCCCGCGCCACCTT
CTCTAGGCACCGGTTCAATTGCCGACCCCTCCCCCCAACTTCTCGGGGACTGTGGGCGATGTGCGCTCTGCCCACTGACGGGC
ACCGGAGCCACTCGAGTGGAATTATCACCTCGAGTTTAC

Expression vectors contain other elements necessary for gene expression, including, for example, a promoter sequence to initiate transcription of the SOXF transcription factor nucleotide sequence, one or more enhancer sequences, translation initiation sequences, and start and stop codons. Suitable promoter sequences include, without limitation, the elongation factor 1-alpha promoter (EF1a) promoter, a phosphoglycerate kinase-1 promoter (PGK) promoter, a cytomegalovirus immediate early gene promoter (CMV), a chimeric liver-specific promoter (LSP) a cytomegalovirus enhancer/chicken beta-actin promoter (CAG), a tetracycline responsive promoter (TRE), a transthyretin promoter (TTR), a simian virus 40 promoter (SV40), and a CK6 promoter. Other promoters suitable for driving gene expression in mammalian cells that are known in the art are also suitable for incorporation into the expression constructs disclosed herein.

In some embodiments, the promoter is a constitutive promoter, where the transcription of the SOXF transcription factor in the pluripotent stem cells in continuous. In other embodiments, the promoter is an inducible promoter to control and/or regulate SOXF transcription factor expression. An inducible promoter is one that only initiates transcription when induced, e.g., by the presence of an appropriate inducible element or agent. In some embodiments, the inducible element or agent is a drug. Suitable inducible promoter systems that are known in the art, e.g., the tetracycline promoter system activated by tetracycline or its derivative doxycycline, or the inducible pLac promoter activated by lactose or lactose analog IPTG, are suitable for use in the methods disclosed herein.

Introducing a SOXF transcription factor nucleic acid molecule or an expression vector comprising a SOXF transcription factor nucleotide sequence into pluripotent stem cells can be carried out using methods known in the art. In some embodiments, the expression vector is introduced into the pluripotent stem cells via transfection, e.g. carried out by electroporation, calcium-phosphate precipitation, DEAE-dextran transfection and the like. In some embodiments, transfection is transient transfection, i.e., the expression vector is not integrated into the genome of the cells. In some embodiments transfection results in the integration of the nucleotide sequence encoding SOXF transcription factor into the genome of the pluripotent stem cells. In some embodiments, the transfected expression vector is excisable and is removed from the cells following the desired culturing and/or differentiation. In some embodiments, the SOXF transcription factor nucleic acid molecule or an expression vector comprising the same are introduced into the pluripotent stem cells via a delivery vehicle, e.g., nanoparticle delivery vehicle or lipid-based particle delivery vehicle. Any suitable nanoparticle delivery vehicle or lipid-based particle delivery vehicle known in the art (see, e.g., Xiao et al., “Engineering Nanoparticles for Targeted Delivery of Nucleic Acid Therapeutics in Tumor,” Mol. Ther. Meth. Clin. Dev. 12: 1-18 (2019) and Ni et al., “Synthetic Approaches for Nucleic Acid Delivery: Choosing the Right Carriers,” Life 9(3): 59 (2019), which are hereby incorporated by reference in their entirety), can be employed in the methods as described herein.

In some embodiments, the delivery vehicle is a lipid-based particle delivery vehicle. Suitable lipid-based vehicles include cationic lipid based lipoplexes (e.g., 1,2-dioleoyl-3trimethylammonium-propane (DOTAP)), neutral lipids based lipoplexes (e.g., cholesterol and dioleoylphosphatidyl ethanolamine (DOPE)), anionic lipid based lipoplexes (e.g., cholesteryl hemisuccinate (CHEMS)), and pH-sensitive lipid lipoplexes (e.g., 2,3-dioleyloxy-N-[2(sperminecarboxamido)ethyl]-N,N-dimethyl-1-propanaminium trifluoroacetate (DOSPA)). Other suitable lipid-based delivery particles incorporate ionizable DOSPA in lipofectamine and DLin-MC3-DMA ((6Z,9Z,28Z,31Z)-heptatriaconta-6,9,28,31-tetraen-19-yl-4-(dimethylamino) butanoate).

In some embodiments, the delivery vehicle is a polymer-based particle, i.e., a polyplex. Suitable polyplex carriers comprise cationic polymers such as polyethylenimine (PEI), and/or cationic polymers conjugated to neutral polymers, like polyethylene glycol (PEG) and cyclodextrin. Other suitable PEI conjugates to facilitate nucleic acid molecule or expression vector delivery in accordance with the methods described herein include, without limitation, PEI-salicylamide conjugates and PEI-steric acid conjugate. Other synthetic cationic polymers suitable for use as a delivery vehicle material include, without limitation, poly-L-lysine (PLL), polyacrylic acid (PAA), polyamideamine-epichlorohydrin (PAE) and poly[2-(dimethylamino)ethyl methacrylate] (PDMAEMA). Natural cationic polymers suitable for use as delivery vehicle material include, without limitation, chitosan, poly(lactic-co-glycolic acid) (PLGA), gelatin, dextran, cellulose, and cyclodextrin.

Solid, inorganic materials suitable for nanoparticle delivery vehicles to facilitate nucleic acid molecule or an expression vector delivery to cells include gold nanoparticles, calcium phosphate nanoparticles, cadinum (quantum dots) nanoparticles, and iron oxide nanoparticles.

Pluripotent stem cells expressing the SOXF transcription factor are cultured in basal medium suitable to promote cell growth for a period of at least 1, at least 2 days, at least 3 days, at least 4 days, at least 5 days, at least 6 days, at least 7 days, at least 8 days, at least 9 days, at least 10 days, or until the pluripotent stem cells have differentiated into the appropriated differentiated progenitor cells. In some embodiments, the differentiated progenitor cell is characterized by a loss of CD34 expression and loss of CD31 expression. In some embodiments, the differentiated progenitor cell population is characterized by expression of VE-cadherin (VEC) and loss of CD31 expression. In some embodiments, the hemogenic endothelial progenitor cells are characterized by their co-expression of VE-cadherin (VEC) and CD34. In some embodiments, the hemogenic endothelial progenitor cells are further characterized by their lack of CD31 and/or CD73 expression. In some embodiments, the culturing is carried out for about 5 days.

In some embodiments, the culturing is carried out in the presence of basal cell growth media, and in the absence of any known hemogenic endothelial differentiation factors. In some embodiments, culturing is carried out in the presence of one or more hemogenic endothelial differentiation factors, such as, for example, and without limitation vascular endothelial growth factor (VEGF) and a glycogen synthase kinase (GSK) inhibitor.

In some embodiments, culturing is carried out in the presence of Runt-related transcription factor 1 (RUNX1) expression. In some embodiments, RUNX1 expression is induced as described above for SOX17 expression, i.e., via the introduction of an expression vector comprising a nucleotide sequence encoding Runt-related transcription factor. In some embodiments, the nucleotide sequence encodes a Runt-related transcription factor having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 10 (shown below). In some embodiments, the nucleotide sequence encoding the Runt-related transcription factor is the human Runt-related transcription factor genomic sequence. In some embodiments, the nucleotide sequence encoding Runt-related transcription factor is the human RUNX1 mRNA sequence comprising a nucleotide sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 9 (GenBank Accession No. D43967)

| | |
|---|---|
| 1 | catagagcca gcgggcgcgg gcgggacggg cgccccgcgg ccggacccag ccagggcacc |
| 61 | acgctgcccg gccctgcgcc gccaggcact tctttccggg gctcctaggg acgccagaag |
| 121 | gaagtcaacc tctgctgctt ctccttggcc tgcgttggac cttccttttt ttgttgtttt |
| 181 | tttttgtttt tcccctttct tccttttgaa ttaactggct tcttggctgg atgttttcaa |
| 241 | cttctttcct ggctgcgaac ttttccccaa ttgttttcct tttacaacag ggggagaaag |
| 301 | tgctctgtgg tccgaggcga gccgtgaagt tgcgtgtgcg tggcagtgtg cgtggcagga |
| 361 | tgtgcgtgcg tgtgtaaccc gagccgcccg atctgtttcg atctgcgccg cggagccctc |
| 421 | cctcaaggcc cgctccacct gctgcggtta cgcggcgctc gtgggtgttc gtgcctcgga |
| 481 | gcagctaacc ggcgggtgct gggcgacggt ggaggagtat cgtctcgctg ctgcccgagt |
| 541 | cagggctgag tcacccagct gatgtagaca gtggctgcct tccgaagagt gcgtgtttgc |
| 601 | atgtgtgtga ctctgcggct gctcaactcc caacaaacca gaggaccagc cacaaactta |
| 661 | accaacatcc ccaaacccga gttcacagat gtgggagagc tgtagaaccc tgagtgtcat |
| 721 | cgactgggcc ttcttatgat tcttgtttta agattagctg aagatctctg aaacgctgaa |
| 781 | ttttctgcac tgagcgtttt gacagaattc attgagagaa cagagaacat gacaagtact |
| 841 | tctagctcag cactgctcca actactgaag ctgattttca aggctactta aaaaaatctg |
| 901 | cagcgtacat taatggattt ctgttgtgtt taaattctcc acagattgta ttgtaaatat |
| 961 | tttatgaagt agagcatatg tatatattta tatatacgtg cacatacatt agtagcacta |
| 1021 | cctttggaag tctcagctct tgcttttcgg gactgaagcc agttttgcat gataaaagtg |
| 1081 | gccttgttac gggagataat tgtgttctgt tgggacttta gacaaaactc acctgcaaaa |
| 1141 | aactgacagg cattaactac tggaacttcc aaataatgtg tttgctgatc gttttactct |
| 1201 | tcgcataaat attttaggaa gtgtatgaga attttgcctt caggaacttt tctaacagcc |
| 1261 | aaagacagaa cttaacctct gcaagcaaga ttcgtggaag atagtctcca ctttttaatg |
| 1321 | cactaagcaa tcggttgcta ggagcccatc ctgggtcaga ggccgatccg cagaaccaga |
| 1381 | acgttttccc ctcctggact gttagtaact tagtctccct cctcccctaa ccacccccgc |
| 1441 | ccccccccac cccccgcagt aataaaggcc cctgaacgtg tatgttggtc tcccgggagc |
| 1501 | tgcttgctga agatccgcgc ccctgtcgcc gtctggtagg agctgtttgc agggtcctaa |
| 1561 | ctcaatcggc ttgttgtgat gcgtatcccc gtagatgcca gcacgagccg ccgcttcacg |
| 1621 | ccgccttcca ccgcgctgag cccaggcaag atgagcgagg cgttgccgct gggcgccccg |
| 1681 | gacgccggcg ctgccctggc cggcaagctg aggagcggcg accgcagcat ggtggaggtg |
| 1741 | ctggccgacc acccgggcga gctggtgcgc accgacagcc ccaacttcct ctgctccgtg |
| 1801 | ctgcctacgc actggcgctg caacaagacc ctgcccatcg ctttcaaggt ggtggcccta |
| 1861 | ggggatgttc cagatggcac tctggtcact gtgatggctg gcaatgatga aaactactcg |
| 1921 | gctgagctga gaaatgctac cgcagccatg aagaaccagg ttgcaagatt taatgacctc |
| 1981 | aggtttgtcg gtcgaagtgg aagagggaaa agcttcactc tgaccatcac tgtcttcaca |
| 2041 | aacccaccgc aagtcgccac ctaccacaga gccatcaaaa tcacagtgga tgggccccga |
| 2101 | gaacctcgaa gacatcggca gaaactagat gatcagacca agcccgggag cttgtccttt |
| 2161 | gaacctcgaa gacatcggca gaaactagat gatcagacca agcccgggag cttgtccttt |
| 2221 | cacccagccc ccacgcccaa ccctcgtgcc tccctgaacc actccactgc ctttaaccct |
| 2281 | cagcctcaga gtcagatgca ggaggaagac acagcaccct ggagatgtta aggcagaagt |
| 2341 | cagttcttct gtccatccct ctccccagcc aggatagagc tatcttttcc atctcatcct |
| 2401 | cagaagagac tcagaagaaa gatgacagcc ctcagaatgc acgttatgag gaaggcagaa |
| 2461 | tgtgggtctg taattcctcc gtgtcccttc tccccctctg caaaccgtcg taacaataat |
| 2521 | agttcctaac acatgggaca attgtgagga ttaaatgagt tagcctgcag aaatcacttg |
| 2581 | atgcacagca catgggaagc attgtgtgta tttattaatc cttcacaaag tctttgagat |
| 2641 | atatttttat caaatattta gcatggatcc cggtacactt tcaatactta ataaatggtc |
| 2701 | aatgttattc tttttcacta tt |

The runt-related transcription factor protein exists in a number of different isoforms. The isoform encoded by the nucleotide sequence above (i.e., isoform AML1a) has the amino acid sequence of SEQ ID NO: 10 below.

(SEQ ID NO: 10)
MRIPVDASTSRRFTPPSTALSPGKMSEALPLGAPDAGAALAGKLRSGDRS
MVEVLADHPGELVRTDSPNFLCSVLPTHWRCNKTLPIAFKVVALGDVPDG
TLVTVMAGNDENYSAELRNATAAMKNQVARFNDLRFVGRSGRGKSFTLTI
TVFTNPPQVATYHRAIKITVDGPREPRRHRQKLDDQTKPGSLSFSERLSE
LEQLRRTAMRVSPHHPAPTPNPRASLNHSTAFNPQPQSQMQEEDTAPWRC

Another aspect of the present disclosure relates to a preparation of hemogenic endothelial progenitor cells produced in accordance with the methods described herein, and preparations of cells enriched for the produced hemogenic endothelial progenitor cells. The hemogenic endothelial progenitor cells of the preparation are identified by their co-expression of VE-cadherin (VEC) and CD34. The VEC⁺ and CD34⁺ hemogenic endothelial progenitor cell fraction of an enriched preparation produced in accordance with the methods described herein may constitute greater than 30% of the preparation. In some embodiments, the hemogenic endothelial progenitor cells are further characterized by their lack of CD31 or CD73 expression. In another embodiment, the hemogenic endothelial progenitor cell fraction of the preparation constitutes greater than 40% of the preparation. In other embodiments, the hemogenic endothelial progenitor cell fraction of the preparation, identified by their coexpression of VEC and CD34, constitutes >45% of the preparation, >50% of the preparation, >55% of the preparation, >60% of the preparation, >65% of the preparation, >70% of the preparation, >75% of the preparation, >80% of the preparation, >90% of the preparation, >95% of the preparation, or >98% of the preparation.

The cell preparations of the present invention are preferably substantially free of non-hematopoietic lineage contaminant cells. In particular, preparations of hemogenic endothelial progenitor cells are substantially free (e.g., containing less than 20%, 15%, 10%, 9%, 8%, 7%, 6%, 5%, 4%, 3%, 2%, or 1%) of other non-hematopoietic lineage cells. The cell preparations of the present invention containing hemogenic progenitor endothelial cells are also substantially free (e.g., containing less than 20%, 15%, 10%, 9%, 8%, 7%, 6%, 5%, 4%, 3%, 2%, or 1%) of non-differentiated, residual pluripotent cell types, e.g., the preparation is substantially free of cells expressing either OCT4, NANOG, SOX2, or SSEA4, and is substantially free of less differentiated cell lineages, e.g., mesoderm cells identified by MIXL1 expression.

In some embodiments, the hemogenic endothelial progenitor cells of the preparation disclosed herein are mammalian cells, including, for example, but without limitation, human, monkey, rat, or mouse cells. In any embodiment, the hemogenic endothelial progenitor cell preparation is a preparation of human hemogenic endothelial progenitor cells.

Another aspect of the present disclosure relates to methods of treating a subject having a condition mediated by a loss or dysfunction of hematopoietic stem cells, i.e., a subject in need of hematopoietic reconstitution. This method involves administering, to the subject having a condition mediated by a loss of hematopoietic stem cells, the enriched preparation of hemogenic endothelial progenitor cells, or a preparation of cells differentiated from said enriched preparation of hemogenic endothelial progenitor cells under conditions effective to treat the condition.

In some embodiments, the subject in need of hematopoietic reconstitutions is a subject having leukemia, e.g., acute myelogenous leukemia (AML), acute lymphoblastic leukemia (ALL), chronic myelogenous leukemia (CMIL), chronic lymphocytic leukemia (CLL), juvenile myelomonocytic leukemia, Hodgkin's lymphoma, non-Hodgkin's lymphoma, or multiple myeloma. In some embodiments, the subject has severe aplastic anemia, Fanconi's anemia, paroxysmal nocturnal hemoglobinuria (PNH), pure red cell aplasia, amegakaryocytosis/congenital thrombocytopenia, severe combined immunodeficiency syndrome (SCID), Wiskott-Aldrich syndrome, beta-thalassemia major, sickle cell disease, Hurler's syndrome, adrenoleukodystrophy, metachromatic leukodystrophy, myelodysplasia, refractory anemia, chronic myelomonocytic leukemia, agnogenic myeloid metaplasia, familial erythrophagocytic lymphohistiocytosis, solid tumors, chronic granulomatous disease, or mucopolysaccharidoses.

The hemogenic endothelial progenitor cells can be administered to a subject in need thereof in the same manner as a conventional bone marrow transplantation or umbilical cord blood transplantation is carried out.

Another aspect of the present disclosure is directed to a method of producing an enriched preparation of hematopoietic progenitor cells. This method involves providing a population of pluripotent stem cells, and inducing expression of a SOXF transcription factor in pluripotent stem cells of the population, wherein an enriched population of hemogenic endothelial progenitor cells is produced as a result of said inducing. The method further involves discontinuing SOXF transcription factor expression in the population of hemogenic endothelial progenitor cells and culturing the population of hemogenic endothelial progenitor cells under conditions effective to produce an enriched preparation of hematopoietic progenitor cells.

Suitable populations of pluripotent stem cells (e.g., human embryonic stem cells and induced pluripotent stem cells) and methods of inducing SOXF transcription factor expression, i.e., SOX-7 expression, SOX-17 expression, SOX-18 expression, or a combination thereof, in the pluripotent stem cells to produce an enriched population of hemogenic endothelial progenitor cells are described supra. In accordance with this aspect of the disclosure, after the pluripotent stem cells differentiate into a population of hemogenic endothelial progenitor cells, SOXF transcription factor expression is discontinued and the population of hemogenic endothelial progenitor cells are cultured further under conditions suitable for inducing differentiation of the hemogenic endothelial cells into hematopoietic progenitor cells. Such culture conditions are known in the art and described herein. For example, suitable conditions include culturing in a suitable serum free stem cell expansion media in the presence of one or more growth factors, including, but not limited to FMS related receptor tyrosine kinase (Flt) and stem cell factor (SCF).

In some embodiments, the preparation of hematopoietic progenitor cells is a preparation of human hematopoietic progenitor cells. In some embodiments, the preparation of hematopoietic cells produced by the methods described herein are characterized by their expression of CD34. In some embodiments, the hematopoietic progenitor cells of the enriched preparation further express one or more proteins selected from CD105, CD110, CD111, CD117, CD133, CD135, CD150, CD184, CD202b, CD243, CD244, CD271, CD309, CD338, CD34, CD38, CD4, CD48, CD90, and CD93. In some embodiments, the hematopoietic progenitor cells of the enriched preparation further express one or more genes selected from CD44, CD45, CD43, TAL1, ETS1, RUNX1, SPI1, ERG, HOXA5, HOXA9, and HOXA10. In some embodiments, the preparation of hematopoietic progenitor cells produced in accordance with the methods herein are non-adherent progenitor cells.

In some embodiments, the hematopoietic progenitor cells of the preparation exhibit lymphoid cell potential. In some embodiments, the hematopoietic progenitor cells of the preparation exhibit the potential to differentiate into a hematopoetic cells selected from erythrocytes, basophils, eosinophils, neutrophils, monocytes, and lymphocytes. In some embodiments, the hematopoietic progenitor cells of the preparation differentiate into a hematopoetic cells selected from erythrocytes, basophils, eosinophils, neutrophils, monocytes, and lymphocytes.

Another aspect of the present disclosure relates to a preparation of hematopoietic progenitor cells produced in accordance with the methods described herein, and preparations of cells enriched for the produced hematopoietic progenitor cell. The CD34 hematopoietic progenitor cell fraction of an enriched preparation produced in accordance with the methods described herein may constitute greater than 30% of the preparation. In another embodiment, the hematopoietic progenitor cell fraction of the preparation constitutes greater than 40% of the preparation. In other embodiments, the hematopoietic progenitor cell fraction of the preparation constitutes >45% of the preparation, >50% of the preparation, >55% of the preparation, >60% of the preparation, >65% of the preparation, >70% of the preparation, >75% of the preparation, >80% of the preparation, >90% of the preparation, >95% of the preparation, or >98% of the preparation.

The cell preparations of the present invention are preferably substantially free of non-hematopoietic lineage contaminant cells. In particular, preparations of hematopoietic progenitor cells are substantially free (e.g., containing less than 20%, 15%, 10%, 9%, 8%, 7%, 6%, 5%, 4%, 3%, 2%, or 1%) of other non-hematopoietic lineage cells. The cell preparations of the present invention containing hematopoietic progenitor cell are also substantially free (e.g., containing less than 20%, 15%, 10%, 9%, 8%, 7%, 6%, 5%, 4%, 3%, 2%, or 1%) of non-differentiated, residual pluripotent cell types, e.g., the preparation is substantially free of cells expressing either OCT4, NANOG, SOX2, or SSEA4, and is substantially free of less differentiated cell lineages, e.g., mesoderm cells identified by MIXL1 expression and hemogenic endothelial progenitor cells identified by their expression of VEC.

In some embodiments, the hematopoietic progenitor cells of the preparation disclosed herein are mammalian cells, including, for example, but without limitation, human, monkey, rat, or mouse cells.

Another aspect of the present disclosure relates to a method of treating a subject having a condition mediated by a loss of immune cells, e.g., a subject needing immune cell reconstitution. This method involves administering to the subject the enriched preparation of hematopoietic progenitor cells as described herein under conditions effective to treat the condition.

In some embodiments, the subject in need of hematopoietic reconstitutions is a subject having leukemia, e.g., acute myelogenous leukemia (AML), acute lymphoblastic leukemia (ALL), chronic myelogenous leukemia (CMIL), chronic lymphocytic leukemia (CLL), juvenile myelomonocytic leukemia, Hodgkin's lymphoma, non-Hodgkin's lymphoma, or multiple myeloma. In some embodiments, the subject has severe aplastic anemia, Fanconi's anemia, paroxysmal nocturnal hemoglobinuria (PNH), pure red cell aplasia, amegakaryocytosis/congenital thrombocytopenia, severe combined immunodeficiency syndrome (SCID), Wiskott-Aldrich syndrome, beta-thalassemia major, sickle cell disease, Hurler's syndrome, adrenoleukodystrophy, metachromatic leukodystrophy, myelodysplasia, refractory anemia, chronic myelomonocytic leukemia, agnogenic myeloid metaplasia, familial erythrophagocytic lymphohistiocytosis, solid tumors, chronic granulomatous disease, or mucopolysaccharidoses.

The hematopoietic progenitor cells can be administered to a subject in need thereof in the same manner as a conventional bone marrow transplantation or umbilical cord blood transplantation is carried out.

Another aspect of the present disclosure relates to a preparation of human cells derived from a pluripotent stem cell line, wherein at least 60% of the preparation comprises hemogenic endothelial progenitor cells expressing VE-cadherin (VEC) and CD34, where the hemogenic endothelial progenitor cells do not express CD31. In some embodiments, the cells of this preparation comprise an expression vector containing a human SOXF transcription factor gene operatively coupled to an inducible promoter. In some embodiments, the SOXF transcription factor is selected from the group consisting of SOX7, SOX17, and SOX18.

In some embodiments, at least 60% of the preparation comprises hemogenic endothelial progenitor cells expressing VE-cadherin (VEC) and CD34, at least 70% of the preparation comprises hemogenic endothelial progenitor cells expressing VE-cadherin (VEC) and CD34, at least 75% of the preparation comprises hemogenic endothelial progenitor cells expressing VE-cadherin (VEC) and CD34, at least 80% of the preparation comprises hemogenic endothelial progenitor cells expressing VE-cadherin (VEC) and CD34, at least 85% of the preparation comprises hemogenic endothelial progenitor cells expressing VE-cadherin (VEC) and CD34, at least 90% of the preparation comprises hemogenic endothelial progenitor cells expressing VE-cadherin (VEC) and CD34, at least 95% of the preparation comprises hemogenic endothelial progenitor cells expressing VE-cadherin (VEC) and CD34, or greater than 95% of the preparation comprises hemogenic endothelial progenitor cells expressing VE-cadherin (VEC) and CD34.

Another aspect of the present disclosure relates to a kit, where the kit includes reagents suitable for differentiating pluripotent stem cells into hemogenic endothelial progenitor cells and/or hematopoietic stem cells. In some embodiments, the kit comprises a nucleic acid molecule encoding a SOXF transcription factor (e.g., SOX7, SOX17, SOX18, or any combination thereof) and reagents suitable for transfecting a preparation of pluripotent stem cells with said SOXF transcription factor nucleic acid molecule. In some embodiments, the nucleic acid molecule encoding the SOXF transcription factor is an RNA molecule as disclosed supra. In some embodiments, the nucleic acid molecule is an expression vector comprising a nucleotide sequence encoding SOXF transcription factor. Suitable expression vectors are disclosed supra. In one embodiment, the expression vector comprises the SOXF transcription factor genomic sequence or mRNA sequence, or variants thereof, operatively coupled to a drug inducible promoter. In such embodiments, the kit may further comprise a drug or other agent capable of inducing promoter mediated SOXF transcription factor expression from the expression vector.

In some embodiments, the kit further comprises basal cell culture media suitable for hemogenic endothelial progenitor cell growth. In some embodiments, the kit further comprises a media suitable for hematopoietic stem cell differentiation and growth. In some embodiments, the kit further comprises one or more growth factors selected from vascular endothelial growth factor, a glycogen synthase kinase (GSK) inhibitor, a transforming growth factor-β (TGF-β) receptor inhibitor, FMS related receptor tyrosine kinase (Flt), and stem cell factor (SCF).

### EXAMPLES

The examples below are intended to exemplify the practice of embodiments of the disclosure but are by no means intended to limit the scope thereof.

### Materials and Methods for Examples 1-6

Maintenance of hPSCs. Human embryonic stem cells (H9, SOX17-mCherry H9 (E. S. Ng, et al., “Differentiation of human embryonic stem cells to HOXA+ hemogenic vasculature that resembles the aorta-gonad-mesonephros,” Nat. Biotechnol. 34:1168-1179 (2016), which is hereby incorporated by reference in its entirety), OCT4-GFP H1) and induced pluripotent stem cells (6-9-9) were maintained on either Matrigel (Corning) or iMatrix-511 silk (Nacalai USA) coated plates in LaSR or mTeSR1 (Stemcell Technologies) pluripotent stem cell medium according to previously published methods (X. Bao, et al., “Chemically-defined albumin-free differentiation of human pluripotent stem cells to endothelial progenitor cells,” Stem Cell Res. 15:122-129 (2015); X. Lian, et al., “A Small Molecule Inhibitor of Src Family Kinases Promotes Simple Epithelial Differentiation of Human Pluripotent Stem Cells,” PLoS One 8(3):e60016 (2013), which are hereby incorporated by reference in their entirety). H9+XLone-SOX17 cells were maintained with g/mL blasticidin (Sigma) to prevent construct silencing. All drugs were removed upon initiating differentiation or forward programming. Cells were routinely tested to ensure Mycoplasma free culture conditions using an established PCR based detection method (L. Young et al., “Detection of Mycoplasma in cell cultures,” Nat. Protoc. 5:929-934 (2010), which is hereby incorporated by reference in its entirety). Cell line details are included in Table 1.

TABLE 1
Cell Lines

| Cell Line Name | Source | Sex | Cell Type |
|---|---|---|---|
| H9 | WiCell | F | hESC |
| 6-9-9 | WiCell | M | iPSC |
| H9 SOX17- | a generous gift | F | Genetically modified |
| mCherry | from Ed Stanley. | | knock in reporter hESC |
| Hl OCT4-GFP | WiCell | M | Genetically modified |
| | | | knock in reporter hESC |

Endothelial progenitor differentiation of hPSCs. Endothelial progenitor differentiation of hPSCs was initiated when hPSCs seeded on Matrigel or iMatrix-511 silk coated plates reached 60% confluence in the presence of Y27632 (Cayman Chemical). Differentiation was performed according to previously published methods (X. Lian, et al., “Efficient Differentiation of Human Pluripotent Stem Cells to Endothelial Progenitors via Small-Molecule Activation of WNT Signaling,” Stem Cell Reports 3, 804-816 (2014), X. Bao, et al., “Chemically-defined albumin-free differentiation of human pluripotent stem cells to endothelial progenitor cells,” Stem Cell Res. 15, 122-129 (2015), which are hereby incorporated by reference in their entirety). Briefly, at day 0, cells were treated with 6 μM CHIR99021 (Cayman Chemical) for 48 hours in LaSR Basal medium, which consists of Advanced DMEM/F12, 2.5 mM GlutaMAX, and 60 μg/ml ascorbic acid, with media refreshed after 24 hours. From day 2-5 cells were maintained in LaSR Basal medium with 50 ng/ml VEGF. Analysis was performed on D5. For forward programming experiments, Dox was added from DO-D2 at 1 μg/mL and from D3-D5 at 5 μg/mL. Cells were replated on iMatrix-511 coated plates on D2 by dissociation with Accutase (Innovative Cell Technologies) for 5 minutes at 37° C., pelleting, and resuspension in the D2 media with 5 μM Y27632.

Hematopoietic progenitor differentiation of hPSCs. On day 5, cells were re-plated by dissociation with Accutase for 5 minutes at 37° C., pelleting, and resuspension in LaSR basal medium with 5 μM Y27632. On D6, media was changed with StemLine II media (Sigma). On D8, the media volume was doubled with fresh media. On D10, the top half of the media was very carefully aspirated and replaced with fresh media. SCF and FLT3L were optional from D6 to D10.

Single cell RNA sequencing. H9 cells were differentiated using endothelial progenitor differentiation protocol described above. On day 5 of differentiation, cells were treated with Accutase for 10 minutes. Single cells were counted and resuspended in PBS with 0.04% BSA. A cell strainer was used to get rid of debris and clumps of cells. The single cell library was constructed using the Chromium Next GEM Single Cell 3′ protocol. Then the library was sequenced on a NextSeq 550 equipment with the High Output 150 cycle kit. scRNA sequencing data was processed through the 10× Genomics Cell Ranger pipeline to generate count matrices. These count matrices were then analyzed using Seurat version 3.2.1 (A. Butler, et al., “Integrating single-cell transcriptomic data across different conditions, technologies, and species,” Nat. Biotechnol. 36:411-420 (2018); T. Stuart, et al., “Comprehensive Integration of Single-Cell Data,” Cell 177:1888-1902 (2019), which are hereby incorporated by reference in their entirety). Briefly, quality control filters were applied to sort out dying or dead cells and multiplets. The gene expression for each cell was then normalized by the total expression, scaled and log transformed. A linear transform was then applied to the data prior to dimensional reduction via PCA analysis. Statistical (JackStraw) and heuristic (elbow plot) strategies were used to determine the number of principle components to include.

Immunostaining. Cells were fixed with 4% formaldehyde (Sigma) for 15 min at room temperature. Cells were washed 3 times with PBS and then blocked for 1 hour at room temperature with DPBS with 0.4% Triton X-100 and 5% non-fat dry milk (BioRad). Cells were stained with primary and secondary antibodies (Table 2) in DPBS with 0.4% Triton X-100 and 5% non-fat dry milk. Nuclei were stained with Hoechst 33342 (Thermo Fisher Scientific). A Nikon TI Eclipse epifluorescence microscope was used for image capture and analysis. Fiji and Matlab were used for further analysis and quantification.

TABLE 2
Antibodies

| Antibody | Source/Host and Isotype/Clone/Catalog# | Application | |
|---|---|---|---|
| SOX17-APC | R&D Systems/goat IgG/IC1924A | 1:50 | (IF/FC) |
| VE-Cadherin | Santa Cruz/mouse IgG₁/Clone: F-8/sc-9989 | 1:100 | (IF/FC) |
| | | 1:1000 | (WB) |
| CD144-FITC | MACS/recombinant human IgG₁/Clone REA199/130-100-742 | 1:100 | (FC) |
| CD34-FITC | MACS/mouse IgG2a/Clone AC 136/130-113-178 | 1:100 | (FC) |
| CD34-APC | MACS/mouse IgG2a/Clone AC 136/130-113-176 | 1:100 | (FC) |
| SOX17 | R&D Systems/rabbit IgG/IC1924A | 1:20000 | (WB) |
| β-actin-HRP | Cell Signaling Technology/rabbit/Clone: 13E5/5125S | 1:20000 | (WB) |
| CD31-APC | MACS/mouse/Clone: AC128/130-092-652 | 1:100 | (IF/FC) |
| CD73-PE | Biolegend/mouse IgG1/Clone: AD2/344003 | 1:100 | (FC) |
| vWF | Santa Cruz/mouse IgG1/Clone: F8/86/sc-53466 | 1:200 | (IF) |
| I-CAM1 | DSHB/mouse IgG1/p2a4 | 1:300 | (IF) |
| FLAG | Sigma-Aldrich/rabbit IgG/F7425 | 1:500 | (FC) |
| CD45-APC | Biolegend/Clone 2D1/368511 | 1:20 | (FC) |
| CD44-FITC | Biolegend/Clone BJ18/338803 | 1:20 | (FC) |

| | | |
|---|---|---|
| Secondary | Alexa 488 Goat anti Ms IgG/A-11029 | 1:1000 |
| Antibody | | |
| Secondary | Anti-mouse IgG HRP-linked/CST 7076S | 1:1000 |
| Antibody | | |
| Secondary | Goat IgG HRP-conjugated/R&D Systems HAF017 | 1:1000 |
| Antibody | | |
| Secondary | Alexa 647 Goat anti Rb IgG/A-21244 | 1:1000 |
| Antibody | | |
| Secondary | Alexa 647 Goat anti Ms IgG/A-21235 | 1:1000 |
| Antibody | | |

Flow cytometry analysis. For staining and analysis of fixed cells, after dissociation with TrypLE Express (differentiated cells) or Accutase (hPSCs), cells were pelleted and resuspended in DPBS with 100 formaldehyde for 30 minutes at room temperature. Cells were pelleted and washed 3 times with DPBS. Cells were stained with primary and secondary antibodies (Table 2) in DPBS with 0.10% Triton X-100 and 0.50% BSA for 2 hours at room temperature. Then cells were pelleted and washed 3 times with DPBS with 0.5% BSA before analysis.

For staining and analysis of live cells, cells were dissociated with TrypLE Express (differentiated cells) or Accutase (hPSCs) and pelleted. For suspension cultures of hematopoietic progenitors, cells were filtered with a 100 μm cell strainer and pelleted. Cells were then resuspended in DPBS with 0.5% BSA and the appropriate conjugated primary antibody dilution and incubated at room temperature for 30 minutes. Cells were pelleted and washed with DPBS with 0.5% BSA. Data were collected on a BD Accuri C6 Plus flow cytometer and analyzed using FlowJo. Gating was based on the corresponding untreated or secondary antibody stained cell control.

Western blotting. Cells were washed with DPBS and lysed with Mammalian Protein Extraction Reagent (Thermo Fisher) with 1× Halt's Protease and Phosphatase (Thermo Fisher) by incubation for 3 minutes. Cell lysate was collected and stored at −80° C. until used. Samples were mixed with Laemmli sample buffer (BioRad) at a working concentration of 1× and incubated at 97° C. for 5 minutes. Samples were loaded into a pre-cast MP TGX stain free gel (BioRad) and run at 200V for 30 minutes in 1× Tris/Glycine/SDS buffer (BioRad). Protein was transferred to a PVDF membrane using a Transblot Turbo Transfer System (BioRad). The membrane was blocked for 30 minutes at room temperature in 1×TBST with 5% Dry Milk. The membrane was incubated overnight at 4° C. with primary antibodies and for 1 hour at room temperature with secondary antibodies (Table 2) in 1×TBST with 5% Dry Milk. The membrane was washed between each antibody exposure with 1×TBST. Chemiluminescence was activated using Clarity Western ECL Substrate (BioRad) and the blot was imaged using a ChemiDoc Touch Imaging System and Image Lab software (BioRad). Blots were analyzed using Fiji software.

Quantitative PCR (qPCR). RNA was extracted from cells using a Direct-zol RNA MiniPrep Plus Kit (Zymo Research R2071). A Maxima First Strand cDNA Synthesis kit (Thermo Fisher K1641) was used to generate cDNA. A BioRad CFX Connect system was used for performing qPCR with PowerSYBR Green PCR Master Mix (Applied Biosystems 4367659) and primers (Table 3). Data was analyzed by the ΔΔCt method where target Ct values were normalized to GAPDH Ct values and fold changes in target gene expression were determined by comparing to day 0 samples. Each sample was run in triplicate. In the event that no measurable expression was detected, relative expression to GAPDH was set to zero.

TABLE 3
qPCR Primers

| Primer | Sequence | SEQ ID NO: |
|---|---|---|
| GAPDH_FWD | 5′-GTGGACCTGACCTGCCGTCT-3′ | SEQ ID NO: 13 |
| GAPDH_REV | 5′-GGAGGAGTGGGTGTCGCTGT-3′ | SEQ ID NO: 14 |
| OCT4_FWD | 5′-CAGTGCCCGAAACCCACAC-3′ | SEQ ID NO: 15 |
| OCT4_REV | 5′-GGAGACCCAGCAGCCTCAAA-3′ | SEQ ID NO: 16 |
| SOX2_FWD | 5′-CAAGATGCACAACTCGGAGA-3′ | SEQ ID NO: 17 |
| SOX2_REV | 5′-GTTCATGTGCGCGTAACTGT-3′ | SEQ ID NO: 18 |
| SOX17_FWD | 5′-GGCGCAGCAGAATCCAGA-3′ | SEQ ID NO: 19 |
| SOX17_REV | 5′-CCACGACTTGCCCAGCAT-3′ | SEQ ID NO: 20 |
| GATA2_FWD | 5′-CTCCCAGCTCTACTCCAGG-3′ | SEQ ID NO: 21 |
| GATA2_REV | 5′-GTGGTGTGAGTCGGGGTG-3′ | SEQ ID NO: 22 |
| CD31_FWD | 5′-GCTGACCCTTCTGCTCTGTT-3′ | SEQ ID NO: 23 |
| CD31_REV | 5′-TGAGAGGTGGTGCTGACATC-3′ | SEQ ID NO: 24 |
| VEC_FWD | 5′ -GTGTTCACGCATCGGTTGTT-3′ | SEQ ID NO: 25 |
| VEC_REV | 5′-CAAATGTGTACTTGGTCTGGGT | SEQ ID NO: 26 |
| | G-3′ | |
| CD34_FWD | 5′-CCTAAGTGACATCAAGGCAGAA- | SEQ ID NO: 27 |
| | 3′ | |
| CD34_REV | 5′-GCAAGGAGCAGGGAGCATA-3′ | SEQ ID NO: 28 |
| DLL4_FWD | 5′-GCACTCCCTGGCAATGTACT-3′ | SEQ ID NO: 29 |
| DLL4_REV | 5′-GCCATCCTCCTGGTCCTTAC-3′ | SEQ ID NO: 30 |
| TAL1_FWD | 5′-GGGATCTGATTCTATCGCCCA-3′ | SEQ ID NO: 31 |
| TAL1_REV | 5′-GCTTTCCCCCTTTTTCGCTG-3′ | SEQ ID NO: 32 |
| SPI1_FWD | 5′-GCCCTGCAATGTCAAGGGA-3′ | SEQ ID NO: 33 |
| SPI1_REV | 5′ -GAAGTCCCAGTAATGGTCGCT- | SEQ ID NO: 34 |
| | 3′ | |
| ETS1_FWD | 5′-CATCCACAAGACAGCGGGG-3′ | SEQ ID NO: 35 |
| ETS1_REV | 5′-CTCGTCGGCATCTGGCTTG-3′ | SEQ ID NO: 36 |
| RUNX1_FWD | 5′-GGTTTCGCAGCGTGGTAAAA-3′ | SEQ ID NO: 37 |
| RUNX1_REV | 5′-GCACTGTGGGTACGAAGGAA-3′ | SEQ ID NO: 38 |
| CD43_FWD | 5′-CATTCCTCAGCCAAGAGCCA-3′ | SEQ ID NO: 39 |
| CD43_REV | ′-GCAAGAGCTGGGACCTGAG-3′ | SEQ ID NO: 40 |
| CD45_FWD | 5′-CAACAGTGGAGAAAGGACGC-3′ | SEQ ID NO: 41 |
| CD45_REV | 5′-TGTCCAGAAAGGCAAAGCCA-3′ | SEQ ID NO: 42 |
| LCOR_FWD | 5′-CAGCAATGCTCCGTTGAGAG-3′ | SEQ ID NO: 43 |
| LCOR_REV | 5′-GAGGGAAACGGGACCTACAC-3′ | SEQ ID NO: 44 |
| ERG_FWD | 5′-CTTGGTCGGAATGGGGAGAG-3′ | SEQ ID NO: 45 |
| ERG_REV | 5′-TGTTCAGAACCTGACGGCTTT-3′ | SEQ ID NO: 46 |
| HOXA5_FWD | 5′-GCTGCACATAAGTCATGACAAC | SEQ ID NO: 47 |
| | A-3′ | |
| HOXA5_REV | 5′-CGCTCAGATACTCAGGGACG-3′ | SEQ ID NO: 48 |
| HOXA9_FWD | 5′-CCCATCGATCCCAATAACCC-3′ | SEQ ID NO: 49 |
| HOXA9_REV | 5′-TGTGGCCTGAGGTTTAGAGC-3′ | SEQ ID NO: 50 |
| HOXA10_FWD | 5′-TACTTCCGCCTTTCTCAGGC-3′ | SEQ ID NO: 51 |
| HOXA10_REV | 5′-CCTTTGGAATTGCCCAGGGA-3′ | SEQ ID NO: 52 |

Generation of H9 XLone-SOXF cells. The open reading frame for human SOX17 was PCR amplified using GoTaq Master Mix (Promega) from the PB-TRE3G-SOX17 plasmid (Table 4). The amplicon was gel purified and ligated into XLone, which was linearized using restriction enzymes KpnI and SpeI (New England Biolabs), using In-Fusion ligase (TaKaRa Bio). XLone-SOX7 and XLone-SOX18 were cloned into XLone by Genewiz. To generate transgenic cell lines, hPSCs were dissociated with Accutase for 10 minutes at 37° C. and pelleted. The cell pellet was resuspended in 100 μL PBS with 8 μg of plasmid DNA, including 3 μg EFla-hyPBase and 5 μg XLone-SOX17 (Table 4). The mixture was transferred to a cuvette and nucleofected using the CB150 program on the Lonza 4D Nucleofector. All plasmid DNA used was prepared using an Invitrogen PureLink HiPure Plasmid Filter Midiprep Kit. Cells were plated at a high density with 5 μM Y27632. Successfully modified cells were purified using media supplemented with 30 μg/mL blasticidin. Upon achieving a relatively pure population, cells were maintained in media containing 20 μg/mL blasticidin. All plasmids generated have been submitted to Addgene.

TABLE 4
Plasmids Used

| Plasmid | Addgene # |
|---|---|
| XLone | 96930 |
| XLone-SOX17 | Pending |
| XLone-SOX7 | Pending |
| XLone-SOX18 | Pending |
| XLone-ETV2 | Pending |
| XLone-Casl3d-SOX17- | Pending |
| gRNA | |
| PB-TRE3G-SOX17 | 104541 |
| EF1a-hyPBase | NA |

Hematopoietic Colony Forming Unit Assay. 5×10 3 Floating cells collected at different time points (day 8, day 10, and day 12) were grown in 1 mL of cytokine containing MethoCult H4434 medium (StemCell Technologies, Vancouver) at 37° C. After 14 days, the hematopoietic colonies were scored for colony-forming units (CFUs) according to cellular morphology.

Giemsa Staining. Day 10 floating cells were collected and methanol fixed on a glass slide. The slide was then stained for 60 minutes at room temperature in a 1:20 dilution of Giemsa stain solution (Sigma-Aldrich). Cells were then washed and mounted for imaging.

Statistics. Experiments were performed in triplicate. Data obtained from multiple experiments or replicates are shown as the mean±standard error of the mean. Where appropriate, one or two tailed Student's t test or ANOVA was utilized (alpha=0.05) with a Bonferroni or Tukey's post hoc test where appropriate. Data were considered significant when p<0.05. Statistical tests were performed using MATLAB or GraphPad Prism.

### Example 1—Single Cell RNA Sequencing Reveals SOXF Factors Expression in Hemogenic Endothelial Progenitors

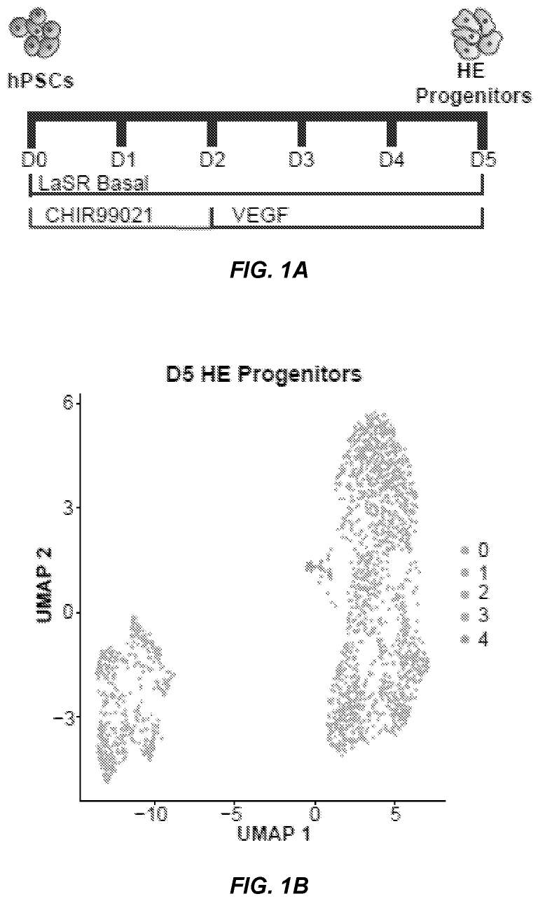

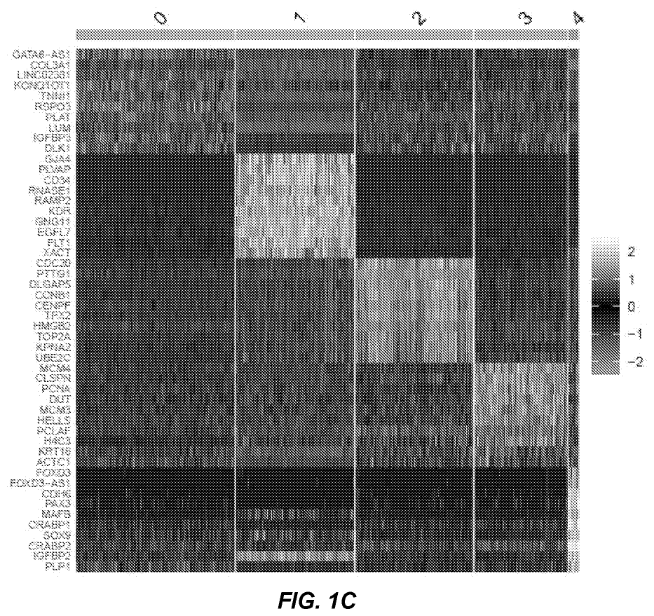

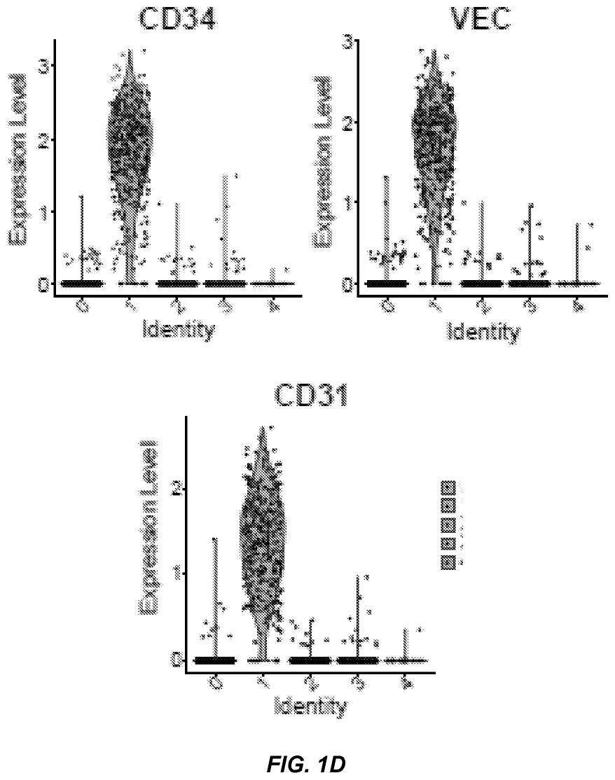

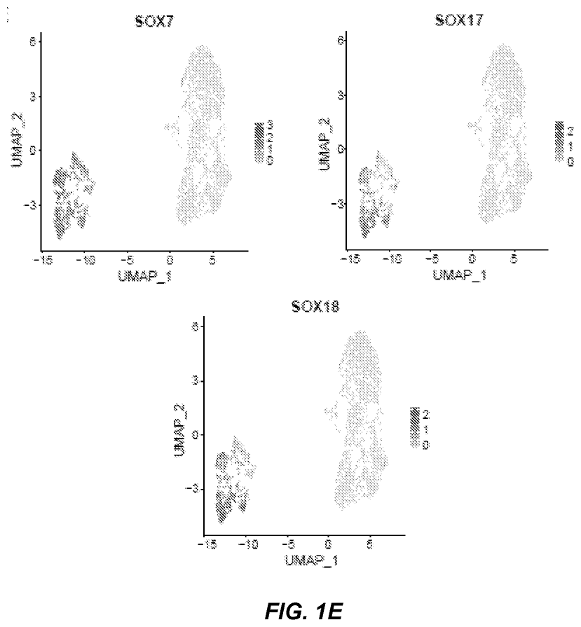

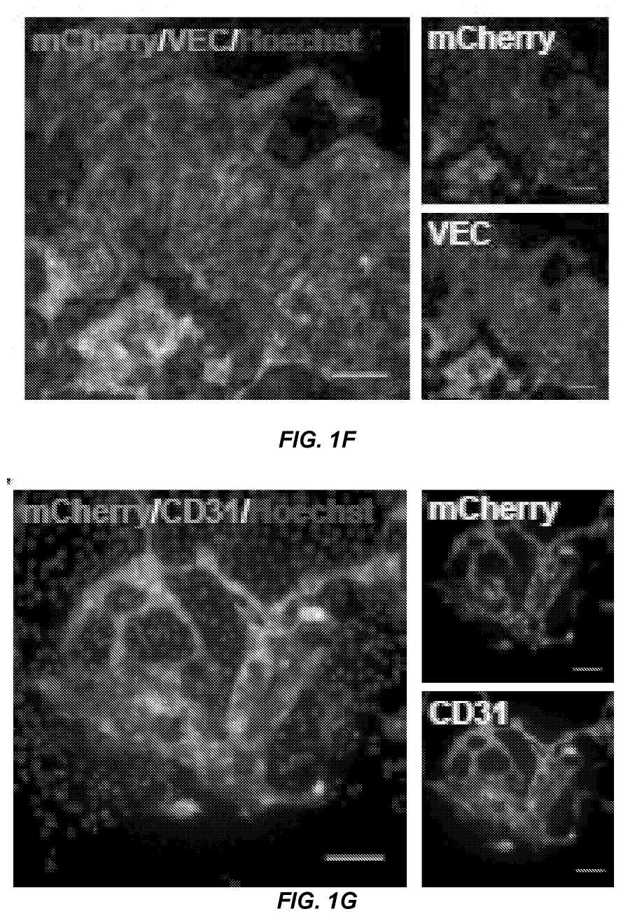

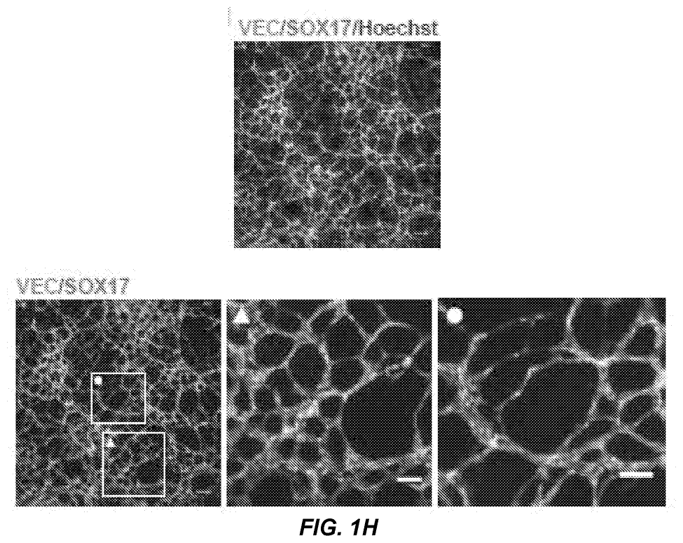

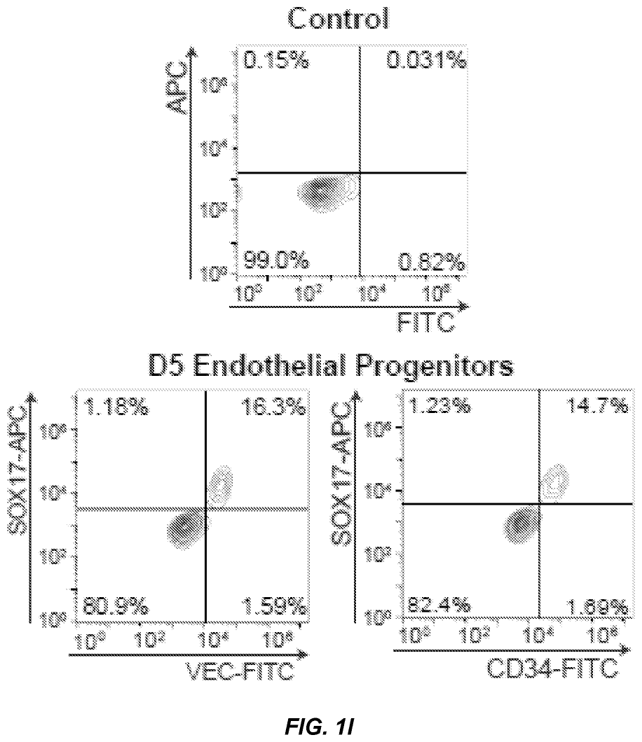

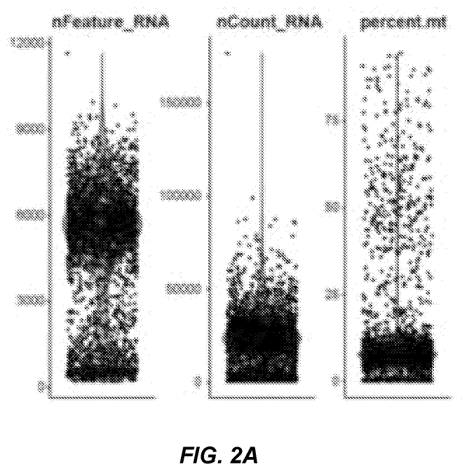

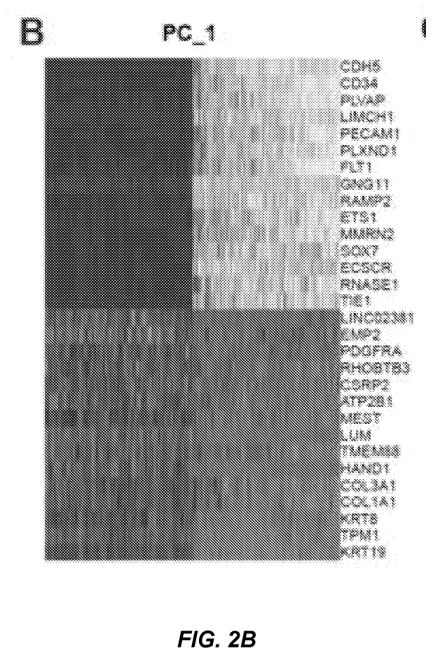

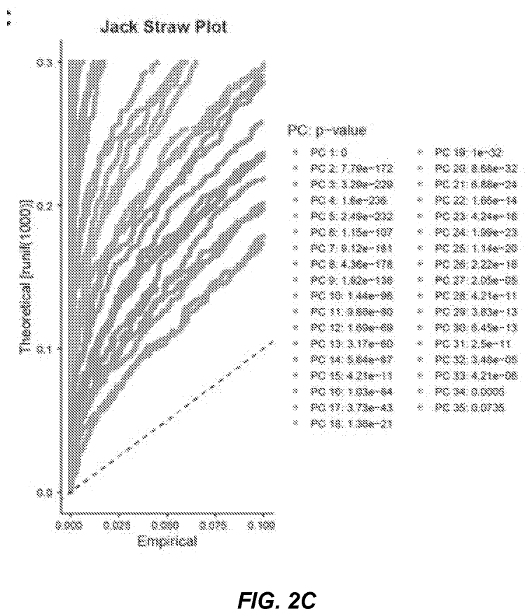

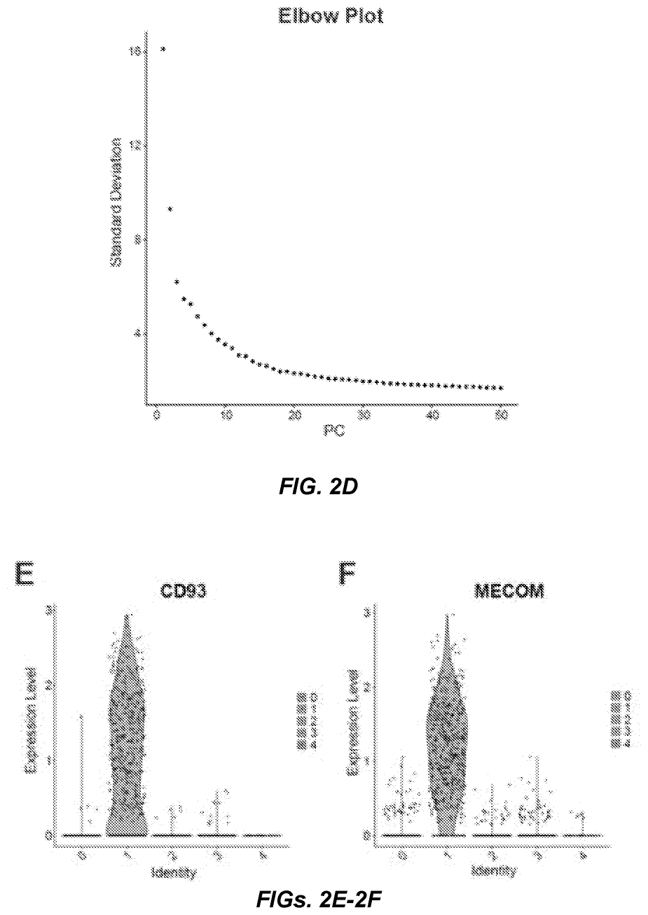

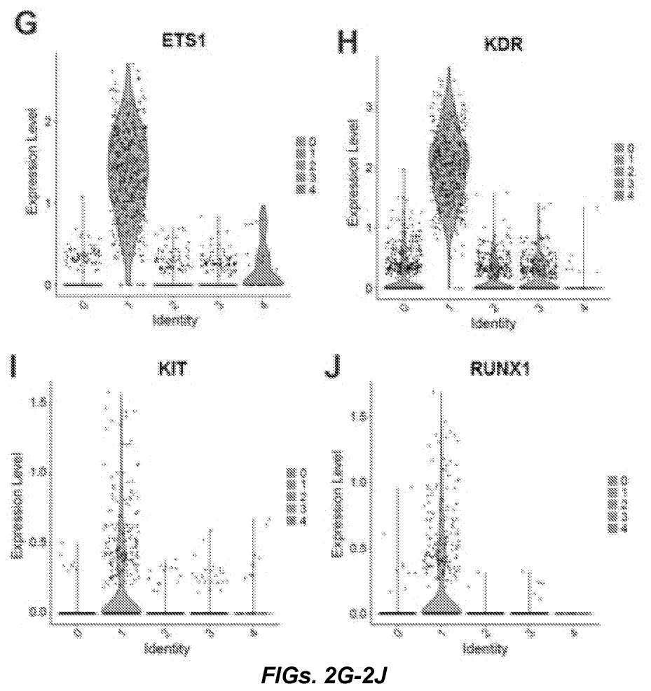

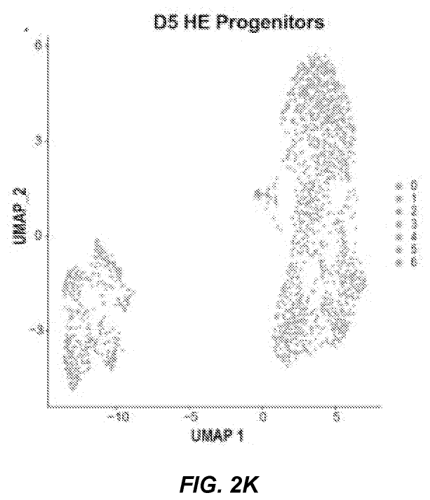

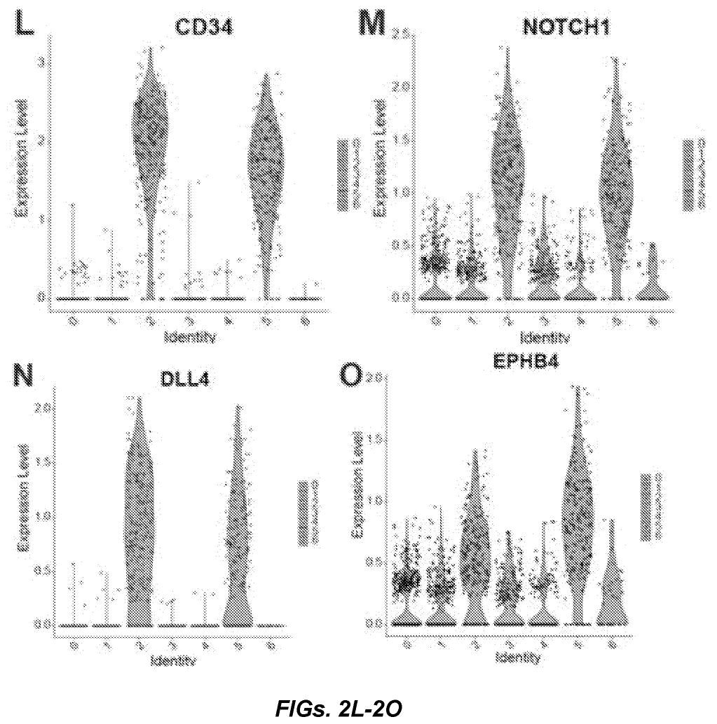

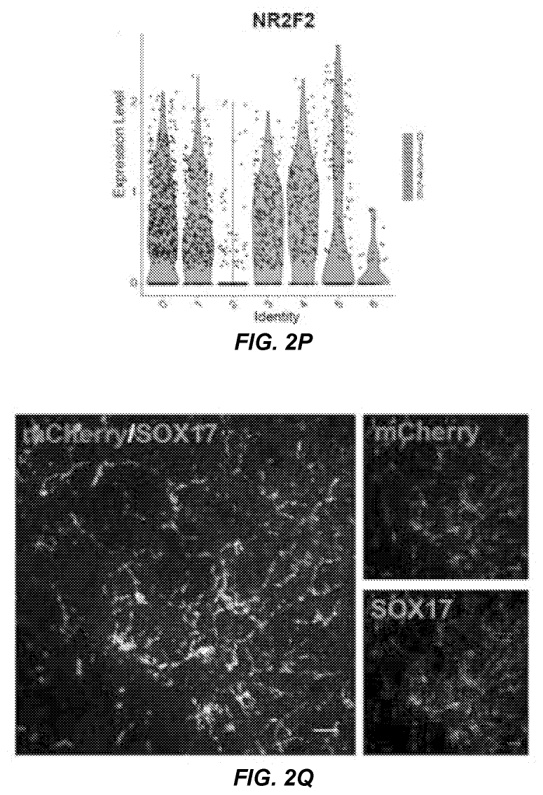

A protocol to generate endothelial progenitors using an initial pulse of Wnt/β-catenin signaling activation with a GSK30 inhibitor, CHIR99021 (CH) was previously developed (X. Lian, et al., “Efficient Differentiation of Human Pluripotent Stem Cells to Endothelial Progenitors via Small-Molecule Activation of WNT Signalling,” Stem Cell Reports 3:804-816 (2014); X. Bao, et al., “Chemically-defined albumin-free differentiation of human pluripotent stem cells to endothelial progenitor cells,” Stem Cell Res. 15:122-129 (2015); L. N. Randolph et al., “Sex-dependent VEGF expression underlies variations in human pluripotent stem cell to endothelial progenitor differentiation,” Sci. Rep. 9:1-8 (2019), which are hereby incorporated by reference in their entirety). These progenitors express CD34, CD31, and VE-Cadherin (VEC), generate primitive vascular structures, and have recently been shown to give rise to hematopoietic cells (X. Lian, et al., “Efficient Differentiation of Human Pluripotent Stem Cells to Endothelial Progenitors via Small-Molecule Activation of WNT Signalling,” Stem Cell Reports 3, 804-816 (2014); Y. Galat, et al., “Cytokine-free directed differentiation of human pluripotent stem cells efficiently produces hemogenic endothelium with lymphoid potential,” Stem Cell Res. Ther. 8:67 (2017), which are hereby incorporated by reference in their entirety). To further establish if this differentiation protocol results in HE cells and identify TFs enriched in the HE population, single cell transcriptome analysis of day 5 differentiated cells was performed (FIG. 1A). 2,673 cells were sequenced and after quality control filters were applied, 1,917 cells were included in the analysis (FIG. 2A). Dimensional reduction and supervised clustering showed five distinct clusters of cells on day 5 of differentiation (FIGS. 1B-C and 2B-D). Clusters (0, 1, 2, 3, and 4) were composed of 609, 457, 454, 358, and 39 cells respectively. Based on the top 100 differentially expressed genes, cluster 0 likely represents cardiac progenitors with upregulation of TNNI1, HAND1, and TMEM88 (Palpant et al., “Transmembrane protein 88: A Wnt regulatory protein that specifies cardiomyocyte development,” Development 140:3799-3808 (2013), which is hereby incorporated by reference in its entirety) (FIG. 1C, Table 5). Cluster 3 cells show differential expression of MYL7, MYL9, and HAND1, and may be, therefore, labeled as atrial cardiac progenitors (Table 5). Cluster 4 has increased expression of SOX2 and POU5F1 and may represent residual undifferentiated hPSCs (Table 5). Cluster 1 separated further away from the other four clusters in the UMAP projection and showed increased expression of CD34, CDH5 (VEC), and PECAM1 (CD31), indicating their endothelial progenitor identity. Other genes that have been identified as markers of the HE population or early hematopoietic lineages, including CD93, MECOM, ETS1, KDR, KIT, and RUNX1, showed increased expression in cluster 1 as compared with all other clusters (FIGS. 2E-J) suggesting their HE identity (A. Ditadi, et al., “Human definitive haemogenic endothelium and arterial vascular endothelium represent distinct lineages,” Nat. Cell Biol. 17:580-91 (2015); F. Anjos-Afonso, et al., “CD34⁻ cells at the apex of the human hematopoietic stem cell hierarchy have distinctive cellular and molecular signatures,” Cell Stem Cell 13:161-174 (2013); M. Maicas, et al., “The MDS and EVI1 complex locus (MECOM) isoforms regulate their own transcription and have different roles in the transformation of hematopoietic stem and progenitor cells,” Biochim. Biophys. Acta—Gene Regul. Mech. 1860:721-729 (2017); M. A. Park, et al., “Activation of the Arterial Program Drives Development of Definitive Hemogenic Endothelium with Lymphoid Potential Article Activation of the Arterial Program Drives Development of Definitive Hemogenic Endothelium with Lymphoid Potential,” Cell Reports 23:2467-2481 (2018); C. M. Sturgeon, et al. “Wnt signaling controls the specification of definitive and primitive hematopoiesis from human pluripotent stem cells,” Nat. Biotechnol. 32:554-561 (2014); A. Ivanovs, et al. “Identification of the niche and phenotype of the first human hematopoietic stem cells,” Stem Cell Reports 2:449-456 (2014), which are hereby incorporated by reference in their entirety). While increased resolution further separated cluster 1 into two clusters, distinct segregation of HE markers or type of endothelial cells were not observed (FIG. 2K). Both groups showed increased expression of DLL4 and NOTCH1, indicative of arterial type endothelial cells, as well as CD34 expression, although CD34 was expressed at higher levels in one of the two sub-clusters. (FIGS. 2L-N). Venous endothelial gene expression was also observed in both clusters although was higher in cluster 5 (FIGS. 2O-P).

TABLE 5

| | Cluster 0 | Cluster 1 | Cluster 2 | Cluster 3 | Cluster 4 |
|---|---|---|---|---|---|
| 1 | GATA6-AS1 | GJA4 | CDC20 | DTL | TFAP2A |
| 2 | COL3A1 | PLVAP | PTTG1 | CDC6 | CCDC140 |
| 3 | MDK | CD34 | KIF20A | MCM10 | FOXD3 |
| 4 | QPRT | RNASE1 | CDKN3 | MCM4 | FOXD3-AS1 |
| 5 | LINC02381 | CDH5 | DLGAP5 | CLSPN | TFAP2B |
| 6 | KCNQ1OT1 | SOX7 | BIRC5 | FEN1 | DLX5 |
| 7 | COL1A1 | LIMCH1 | CCNB2 | CHAF1A | WNT1 |
| 8 | HNRNPA1 | KLF2 | CCNB1 | PCNA | CDH6 |
| 9 | BNC2 | CALCRL | CENPF | DUT | CT75 |
| 10 | TNNI1 | PECAM1 | TUBB4B | PSMC3IP | SOX2 |
| 11 | CRB2 | F2RL2 | H2AZ1 | GINS2 | ZIC2 |
| 12 | RSPO3 | ECSCR | KIF4A | WDR76 | ESRG |
| 13 | SNHG29 | LMO2 | ARL6IP1 | MCM3 | PAX3 |
| 14 | VIM | TIE1 | TPX2 | BRCA1 | SLIT1 |
| 15 | SPARC | FLI1 | HMGB2 | ORC6 | TFAP2C |
| 16 | COL6A3 | ESAM | NUCKS1 | UHRF1 | MIR205HG |
| 17 | PDGFRA | AFAP1L1 | HMGB1 | HELLS | LAMP5 |
| 18 | MXD4 | MMRN2 | HMMR | MASTL | MAFB |
| 19 | NCAM1 | RASGRP3 | CDCA3 | XRCC2 | SLC7A11 |
| 20 | BCO2 | MYCT1 | UBE2S | TK1 | NOG |
| 21 | NME4 | MECOM | HMGN2 | RAD51 | POU5F1 |
| 22 | PLAT | SOX18 | MKI67 | CDT1 | TTYH1 |
| 23 | AC022168.1 | ETS1 | TOP2A | RMI2 | C11orf96 |
| 24 | DDR2 | ADGRL4 | TUBA1C | ATAD2 | GABRP |
| 25 | KRT19 | FAM124B | CALM2 | DHFR | CRABP1 |
| 26 | GATA6 | CD93 | TACC3 | MCM5 | IGFBPL1 |
| 27 | COL6A1 | TGM2 | CKS1B | RAD51AP1 | PTPRZ1 |
| 28 | APOE | MEF2C | HMGB3 | GMNN | PPP1R1A |
| 29 | LINC01697 | PLXND1 | BUB1 | MCM6 | ENCI |
| 30 | HAND1 | ICAM2 | KPNA2 | ESCO2 | TRMT9B |
| 31 | FNDC5 | CD40 | PLK1 | ASF1B | SOX9 |
| 32 | CPE | F2R | NEK2 | RRM2 | CRABP2 |
| 33 | RELN | RAMP2 | CENPW | MCM7 | ALCAM |
| 34 | TMEM88 | BGN | AURKB | POLD3 | IGFBP5 |
| 35 | CDH3 | VAMP5 | CENPA | DEK | CCDC160 |
| 36 | PLD3 | KDR | CCNA2 | H1-5 | ALPL |
| 37 | SOX6 | PROCR | GTSE1 | FBXO5 | IGFBP2 |
| 38 | CD74 | NOTCH4 | DYNLL1 | CDCA5 | ID4 |
| 39 | SERPINF1 | HLA-E | TUBA1B | ATAD5 | PKDCC |
| 40 | DCN | GNG11 | RAN | PCLAF | ROR1 |
| 41 | MEST | F11R | ANP32E | USP1 | SGK1 |
| 42 | FOXH1 | SOX17 | RANBP1 | ZWINT | TSHZ2 |
| 43 | LUM | EGFL7 | NUF2 | RFC4 | GAS1 |
| 44 | RHOBTB3 | FLT1 | PRR11 | TYMS | MPZ |
| 45 | COL11A1 | IGFBP4 | BUB1B | CDCA4 | METRN |
| 46 | COL6A2 | IFI16 | RAD21 | CHEK1 | PFN2 |
| 47 | FGFR2 | TSPAN13 | MAD2L1 | CDK1 | CCND1 |
| 48 | HOXB3 | TFPI | PIMREG | UBE2T | TUBB2B |
| 49 | ANK3 | HSPG2 | DEPDC1B | SLBP | BTBD3 |
| 50 | DSP | PLK2 | NCAPD2 | MCM2 | NRP2 |
| 51 | RAB38 | KLHL4 | ASPM | WDHD1 | GAPDH |
| 52 | WFIKKN1 | HOPX | CENPE | H4C3 | PKM |
| 53 | PCOLCE | SPTBN1 | PBK | CCNE1 | RND2 |
| 54 | VCAN | SLC9A3R2 | UBE2C | UNG | PLP1 |
| 55 | CDKN1C | LAPTM4B | KNL1 | SRSF7 | SPINT2 |
| 56 | RERG | CLIC1 | CKAP5 | CENPU | PCDH10 |
| 57 | TMEM141 | XACT | HSP90AA1 | BRCA2 | FZD3 |
| 58 | LAPTM4A | DIPK1B | CEP55 | MAD2L1 | SRGAP3 |
| 59 | COL1A2 | NOTCH1 | DEPDC1 | MMS22L | STT3B |
| 60 | MAGED1 | IGFBP2 | KIF2C | RFC2 | CNTNAP2 |
| 61 | IGFBP3 | VAMP8 | KIF22 | NASP | MSX2 |
| 62 | LGR4 | CAVIN1 | NCAPG | CENPK | EDNRB |
| 63 | ANXA6 | PALM2AKAP2 | SFPQ | RRM1 | PRTG |
| 64 | TPM1 | PRCP | STMN1 | SRSF2 | GADD45A |
| 65 | HOXB-AS3 | HEY2 | JPT1 | HNRNPAB | NR6A1 |
| 66 | BMP5 | CCDC85B | HNRNPH3 | MSH6 | TNNT1 |
| 67 | GPRC5C | S100A16 | DTYMK | BARD1 | L1TD1 |
| 68 | HAND2 | TSC22D3 | H2AX | VRK1 | UCHL1 |
| 69 | DDIT4 | LIFR | PSRC1 | DNMT1 | CNKSR3 |
| 70 | PDE3A | ITM2B | ECT2 | DIAPH3 | EPCAM |
| 71 | CSRP2 | RHOC | NASP | DNAJC9 | DST |
| 72 | H19 | SLC2A3 | LMNB1 | HAT1 | TPBG |
| 73 | AHCY | RDX | CDCA8 | RPA3 | MAP1B |
| 74 | PTPN13 | HAPLN1 | KIF14 | MYBL2 | ZFHX4 |
| 75 | POSTN | NEAT1 | KNSTRN | CDCA7 | HMGA2 |
| 76 | ATP2B1 | PDLIM1 | SMC4 | SMC3 | WLS |
| 77 | DSG2 | EFNA1 | SGO2 | PAICS | CLDN6 |
| 78 | TM7SF2 | LIMSI | KIF20B | H1-3 | LIN28A |
| 79 | REC8 | TAGLN2 | MZT1 | RANBP1 | UBL3 |
| 80 | RGS4 | HES4 | DEK | CENPV | NEFM |
| 81 | ZFP36L1 | TLNRD1 | KIF11 | KRT18 | SNAI2 |
| 82 | MSX2 | CD9 | CDCA2 | EMP2 | GPM6B |
| 83 | PRPH | GSN | MORF4L2 | MGST1 | CLU |
| 84 | PKP2 | COL4A2 | DNAJA1 | KRT8 | COL2A1 |
| 85 | ATF7IP2 | PCDH17 | ODC1 | H1-2 | ARL4C |
| 86 | DSC2 | COL4A1 | LBR | RPL22L1 | BBS9 |
| 87 | SNHG7 | DEPP1 | CKS2 | MEST | SUCO |
| 88 | SLIT2 | EFNB2 | TMPO | FABP5 | RHOB |
| 89 | KCTD12 | COTL1 | PCLAF | CSRP2 | LDHA |
| 90 | SYT10 | S100A4 | ANLN | MYL7 | NNAT |
| 91 | IRX3 | FAM107B | CKAP2 | MYL9 | ACP3 |
| 92 | LRRN3 | PTP4A3 | TTK | ACAT2 | SLC2A3 |
| 93 | SOX4 | ARGLU1 | KIF23 | SLC9A3R1 | IFT57 |
| 94 | UCP2 | HEY1 | FABP5 | ID2 | ZFP36L2 |
| 95 | NTRK2 | SAT1 | CCDC34 | PTN | CITED2 |
| 96 | BST2 | ARHGAP29 | NDC80 | HAND1 | DDIT4 |
| 97 | DLK1 | PRSS23 | AURKA | LUM | EDNRA |
| 98 | SERPINB9 | SEC14L1 | TPM1 | TPM1 | ID3 |
| 99 | TECRL | UNC5B | BUB3 | ACTC1 | IFITM3 |
| 100 | PBX3 | BEX1 | CDK1 | ART5 | SLC2A1 |

Inspection of differentially expressed TFs uncovered high expression of all three SOXF factors in the HE cells (FIG. 1E). No other SOX factors were differentially expressed in cluster 1; although differential expression of SOX6 and SOX4 in cluster 0 and SOX2 and SOX9 in cluster 4 were observed (Table 5). To confirm this finding, a SOX17-mCherry knockin reporter hPSC line (E. S. Ng, et al., “Differentiation of human embryonic stem cells to HOXA+ hemogenic vasculature that resembles the aorta-gonad-mesonephros,” Nat. Biotechnol. 34:1168-1179 (2016), which is hereby incorporated by reference in its entirety) was used and the knockin hPSCs to day 5 HE progenitors was differentiated. The reporter cell line was validated by antibody staining on day 5 of differentiation (FIG. 2Q). In addition, mCherry expression only occurred in cells also expressing VEC and CD31 on day 5 of differentiation (FIGS. 1F-G). The co-expression of SOX17 with HE progenitor markers VEC and CD34 was also observed in the HE differentiation of an additional hPSC line (6-9-9) by immunostaining and flow cytometry (FIGS. 1H-I). Furthermore, SOX17 in particular has been identified as a marker of definitive HE cells in both mouse and human models (R. L. Clarke, et al., “The expression of Sox17 identifies and regulates haemogenic endothelium,” Nat. Cell Biol. 15:502-10 (2013); S. Irion, et al., “Temporal specification of blood progenitors from mouse embryonic stem cells and induced pluripotent stem cells,” Dev. Stem Cells 137:2829-2839 (2010); M. Kennedy, et al., “T Lymphocyte Potential Marks the Emergence of Definitive Hematopoietic Progenitors in Human Pluripotent Stem Cell Differentiation Cultures,” Cell Rep. 2:1722-1735 (2012), which are hereby incorporated by reference in their entirety). These data, along with a study (Y. Galat, et al., “Cytokine-free directed differentiation of human pluripotent stem cells efficiently produces hemogenic endothelium with lymphoid potential,” Stem Cell Res. Ther. 8, 67 (2017), which is hereby incorporated by reference in its entirety, in which lymphoid cells were generated from endothelial progenitor cells using our CH-induced HE differentiation protocol) provide strong evidence that this protocol does result in HE cells and our scRNA-seq data revealed upregulated expression of SOXF factors in HE cells.

### Example 2—Overexpression of SOX17 but not SOX7 or SOX18 Enhances Hemogenic Endothelial Differentiation Efficiency from hPSCs

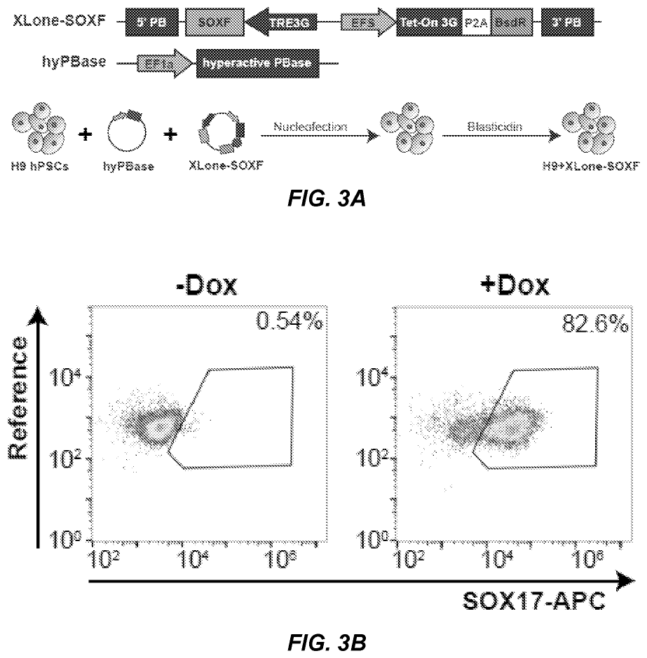

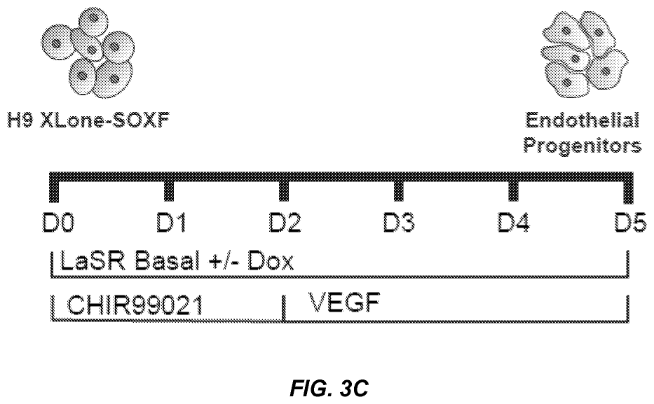

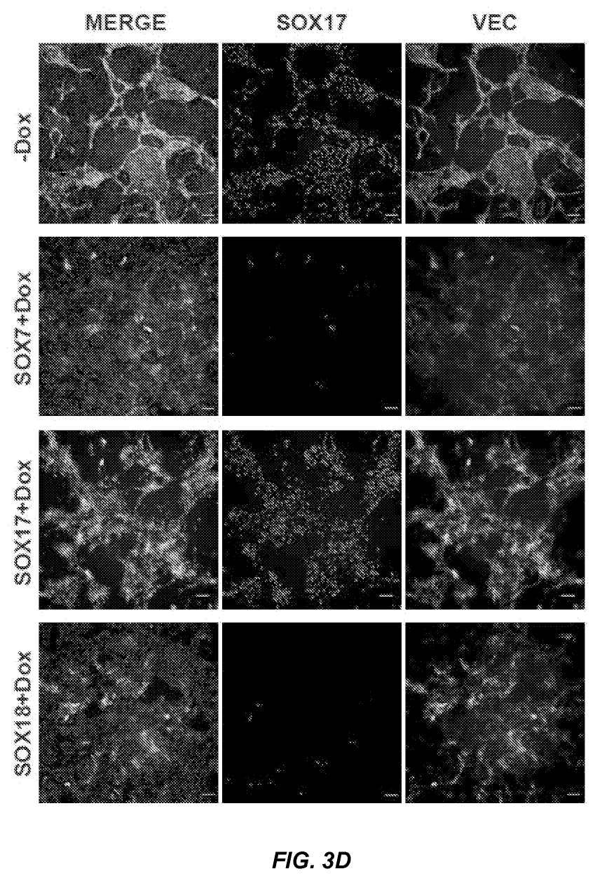

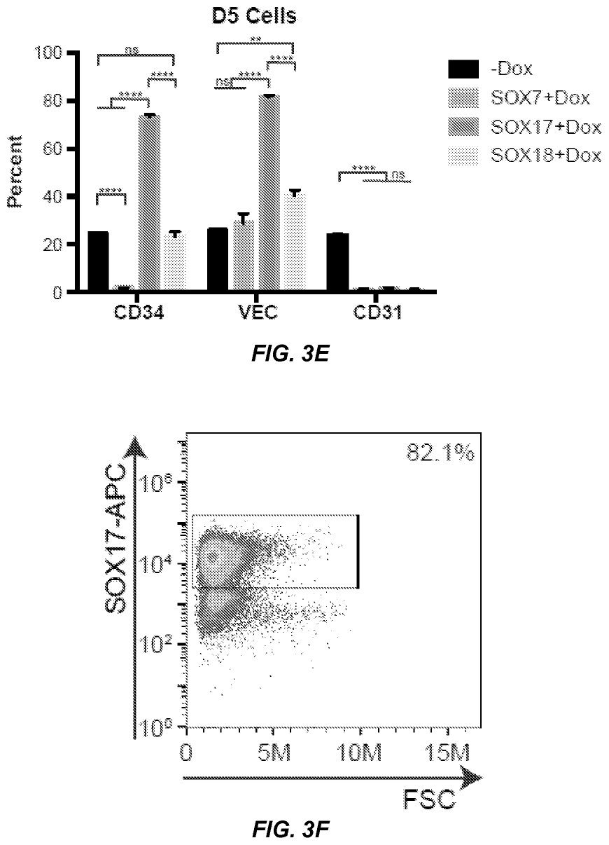

Upon discovering that all 3 SOXF factors are expressed in HE cells, it was desirable to understand which, if any of these factors, play a functional role in determining HE cell fate. To address this question, cell lines with inducible overexpression of SOX7, SOX17, and SOX18 were generated by cloning each TF into our doxycycline (Dox) inducible, PiggyBac-based XLone construct (L. N. Randolph, et al., “An all-in-one, Tet-On 3G inducible PiggyBac system for human pluripotent stem cells and derivatives,” Sci. Rep. 7:1549 (2017), which is hereby incorporated by reference in its entirety) (FIG. 3A). This construct was then introduced into hPSCs, and cells successfully incorporating the construct were purified by drug selection (FIG. 3A). The modified cells were then referred to as H9+XLone-SOX7, H9+Xlone-SOX17, and H9+XLone-SOX18. To ensure the desired function of resulting cells, H9+XLone-SOX17 cells were treated with or without Dox for 24 hours, which revealed robust overexpression of SOX17 up to 82.6%, with minimal leakage in cells not treated with Dox (FIG. 3B). This was consistent with the previous findings and indicates the successful generation of stable transgenic cell lines (L. N. Randolph, et al., “An all-in-one, Tet-On 3G Inducible PiggyBac System for Human Pluripotent Stem Cells and Derivatives,” Sci. Rep. 7:1549 (2017), which is hereby incorporated by reference in its entirety).

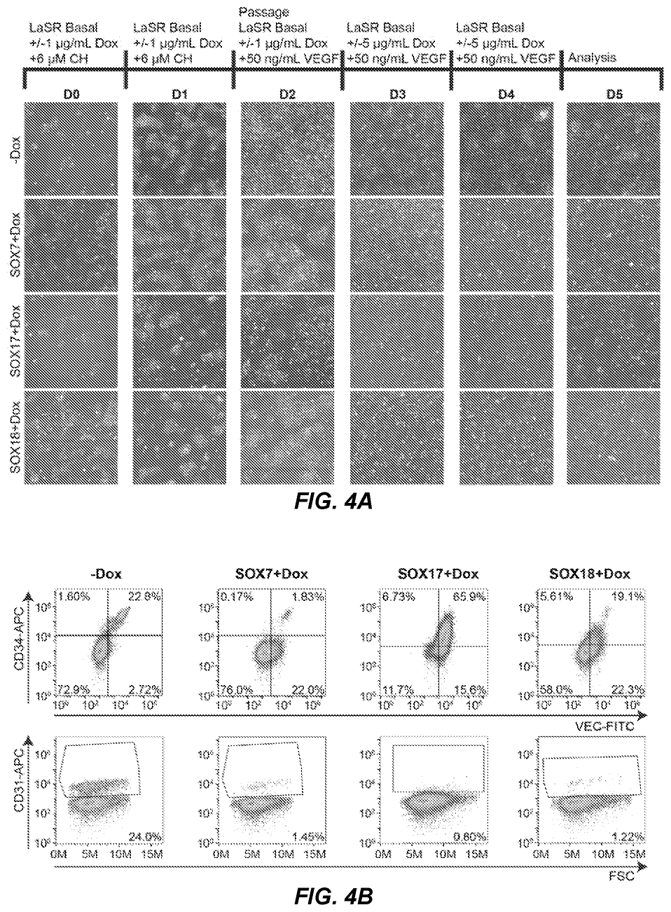

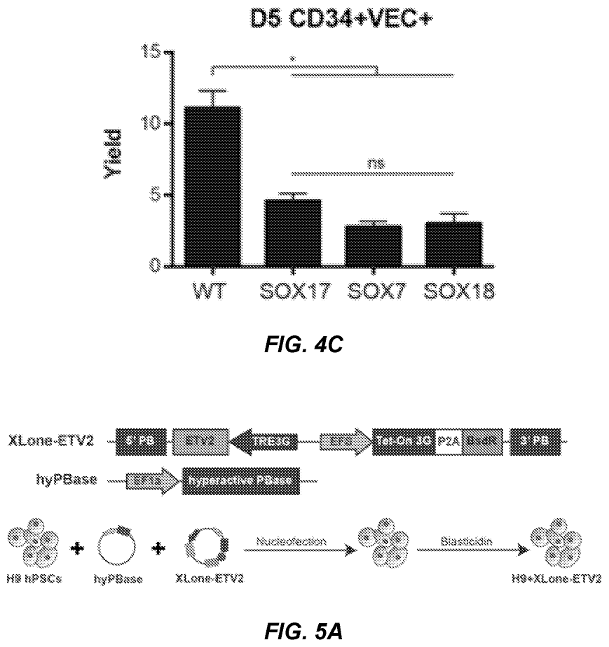

To test whether overexpression of SOXF factors enhances CH-induced HE differentiation from hPSCs, each cell line was differentiated to HE progenitors in the presence or absence of Dox and compared the expression of CD34, VEC, CD31, and SOX17 (FIGS. 3C and 4A). The expression levels of endothelial markers in cells without Dox treatment was consistent with the earlier experiments, and the differentiation efficiency was comparable with the previous results (X. Lian, et al., “Efficient Differentiation of Human Pluripotent Stem Cells to Endothelial Progenitors via Small-Molecule Activation of WNT Signalling,” Stem Cell Reports 3:804-816 (2014); L. N. Randolph, et al., “Sex-dependent VEGF expression underlies variations in human pluripotent stem cell to endothelial progenitor differentiation,” Sci. Rep. 9:1-8 (2019), which are hereby incorporated by reference in their entirety) (FIGS. 3D-E and 4B). For cells differentiated in the presence of Dox, a dramatic increase in the SOX17 population of up to 82% for the H9+XLone-SOX17 cells was observed, as would be expected, and very few SOX17⁺ cells in the day 5 cells generated from either H9+XLoneSOX7 or H9+XLone-SOX18 (FIGS. 3D and 3F). H9+XLone-SOX7 cells also significantly decreased the percentage of CD34⁺ cells with no change in the percentage of VEC⁺ cells (FIGS. 3D-E and 4B). H9+XLone-SOX18 cells showed no change in the CD34⁺ population but a statistically significant increase in the VEC⁺ population (40.27%±2.32%, p=0.0035) (FIGS. 3D-E and 4B). The day 5 cells resulting from H9+XLone-SOX17 cells showed significant increases in CD34+(73.10% 0.94%, p=1.15e-6) and VEC⁺ (81.40%±0.90%, p=4.97e-7) populations over differentiation without any transgene expression (FIGS. 3D-E and 5). Fluorescent microscopy revealed that the majority of SOX17⁺ cells were also VEC⁺ (FIG. 3D). A statistically significant difference in the yields for cells generated with each SOXF factor were not observed; however, there were fewer cells generated than with the normal differentiation protocol (FIG. 4C). Interestingly, overexpression of each SOXF factor resulted in a loss of CD31 expression (FIGS. 3E and 4B). Collectively, this demonstrates that forced overexpression of SOX17, but not SOX7 or SOX18, increases CH-induced HE differentiation efficiency, highlighting a functional role for SOX17 in the acquisition of HE cell fate.

### Example 3—SOX17 Expression Occurs Prior to Hemogenic Endothelial Markers and CRISPR-Cas13d Interference with SOX17 Inhibits HE Differentiation

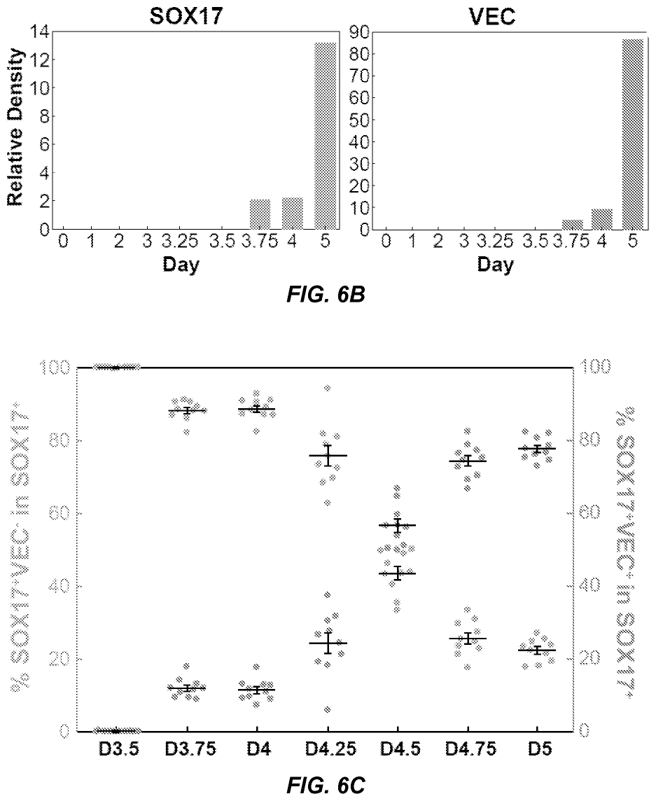

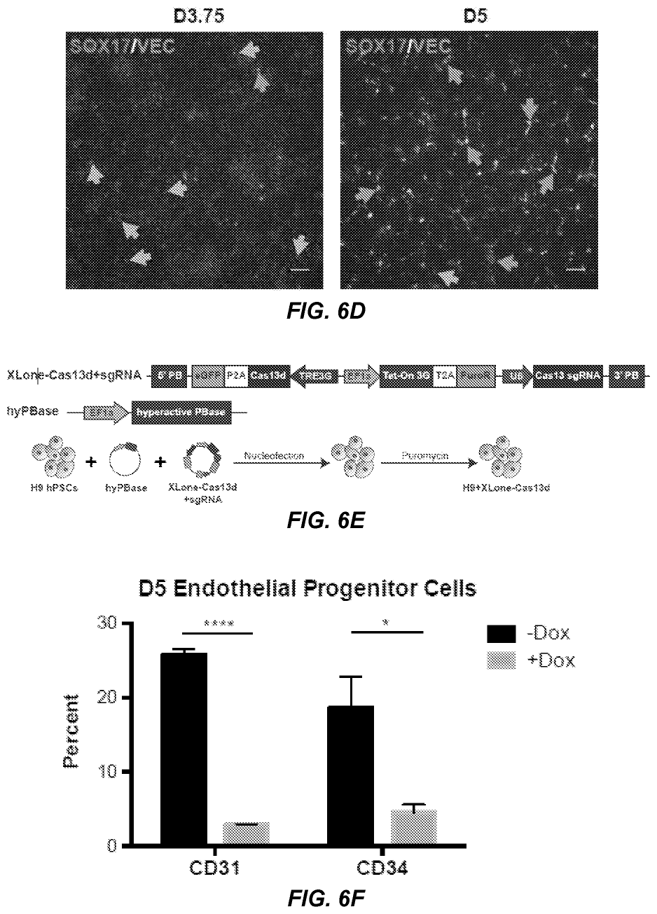

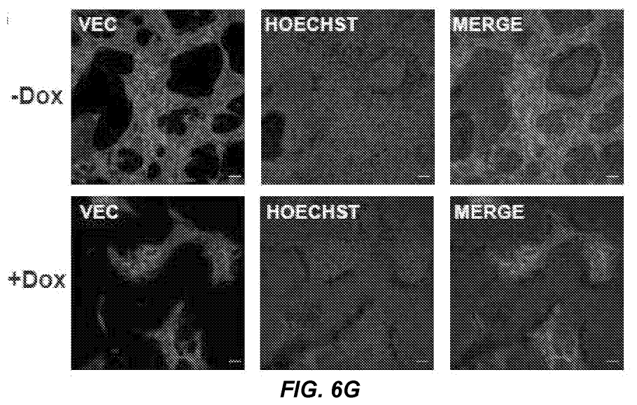

To better understand the role of SOX17 during differentiation, SOX17 expression kinetics along hPSC differentiation to HE cells were characterized. hPSCs was differentiated following the protocol illustrated in FIG. 1A and cells were collected daily until day 3 and every six hours after day 3 until day 5. Western blot analysis showed SOX17 and VEC are first detected on day 3.75 (FIGS. 6A-B and 7A). Immunofluorescent analysis revealed that there are more cells expressing SOX17 alone on day 3.75, and this SOX17 single positive cell number gradually decreases as cells become double positive for SOX17 and VEC over time (FIGS. 6C-D and 7B). This data indicates that expression of SOX17 occurred before the appearance of VEC⁺ HE cells.

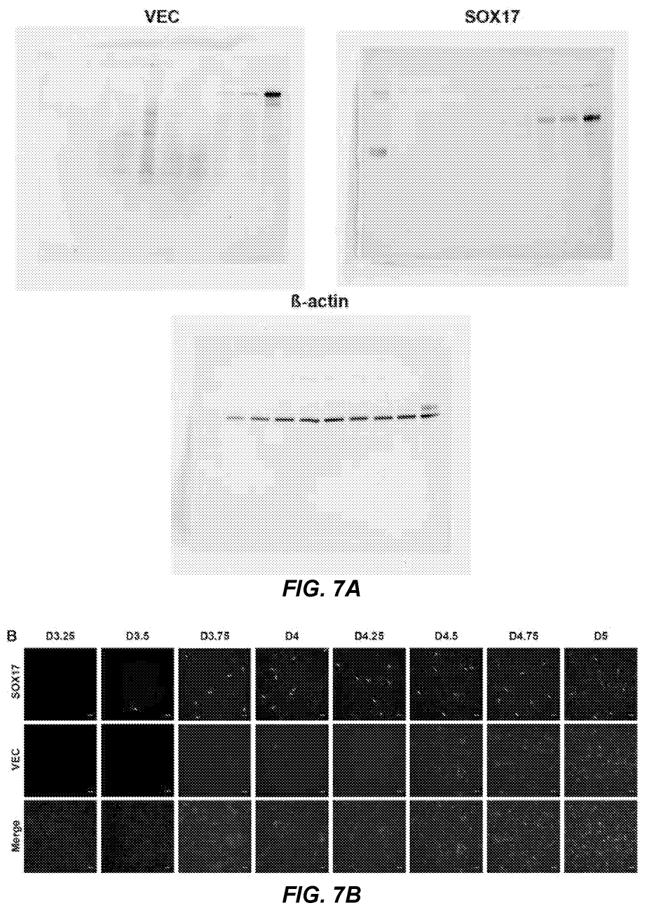

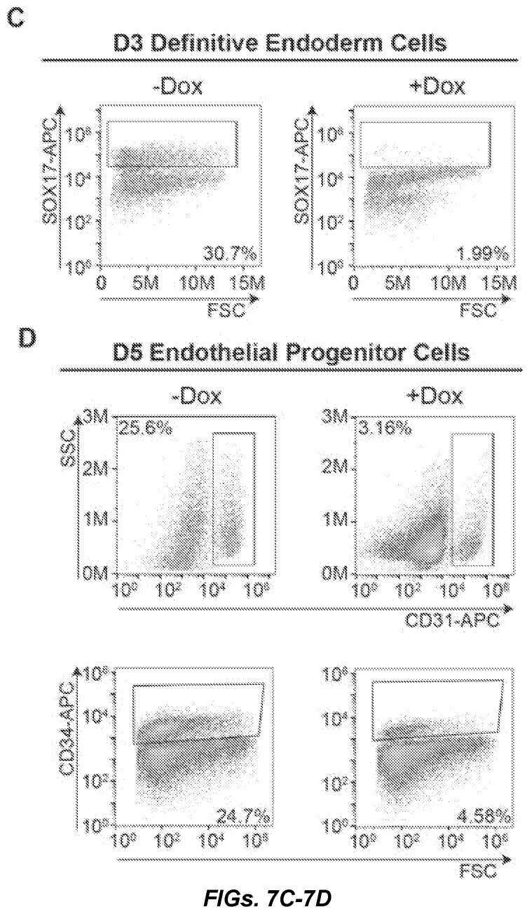

To further study the role of SOX17 in HE differentiation, a loss-of-function analysis was performed using a CRISPR-Cas13d-meditated knockdown approach. This member of the Cas13 family can knockdown coding and non-coding RNA transcripts efficiently (S. Konermann, et al., “Transcriptome Engineering with RNA-Targeting Type VI-D CRISPR Effectors,” Cell 173:665-676 (2018), which is hereby incorporated by reference in its entirety). Cas13d was cloned into other XLone plasmid construct under the control of the inducible TRE3G promoter (FIG. 6E) (L. N. Randolph, X. et al., “An all-in-one, Tet-On 3G inducible PiggyBac system for human pluripotent stem cells and derivatives,” Sci. Rep. 7:1549 (2017), which is hereby incorporated by reference in its entirety). Also a U6 promoter expressing SOX17 gRNA was cloned into the plasmid to establish a single transposon system for Cas13d interference. This plasmid was nucleofected into H9 cells and puromycin drug selection was used to purify the cells that integrated XLone-Cas13d system (FIG. 6E). To test Cas13d-mediated SOX17 knockdown efficiency, definitive endoderm (SOX17⁺ cells) differentiation with or without Dox was performed and this experiment revealed robust and near complete SOX17 knockdown was achieved as measured by flow cytometry analysis (FIG. 7C). HE differentiation of these cells with and without Dox treatment revealed abrogation of CD31 and CD34 expression as well as reduced VEC expression upon SOX17 knockdown (FIGS. 6F-G and 7D). These results demonstrate that SOX17 expression is critical to the formation of HE cells from hPSCs.

### Example 4—Overexpression of SOX17 Alone is Sufficient for the Generation of Cells Expressing CD34 and VEC From hPSCs

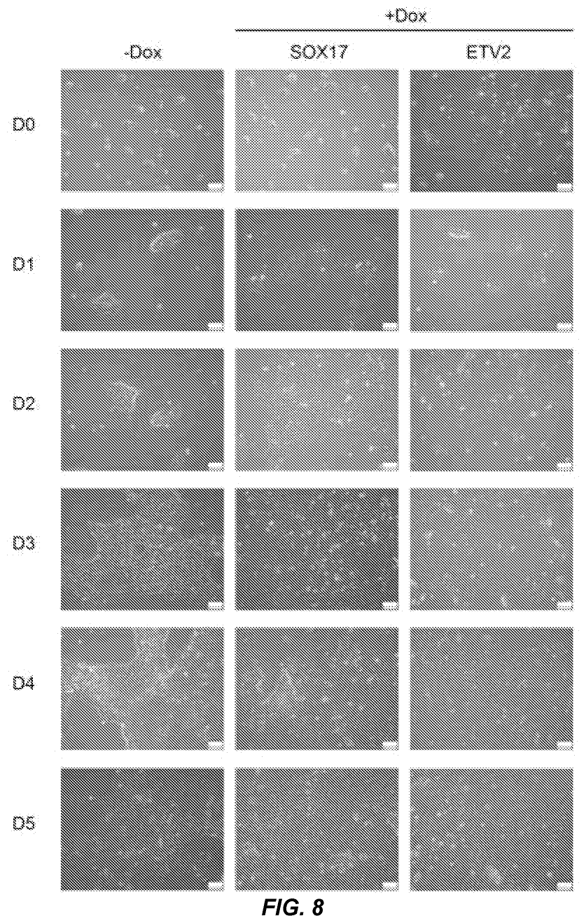

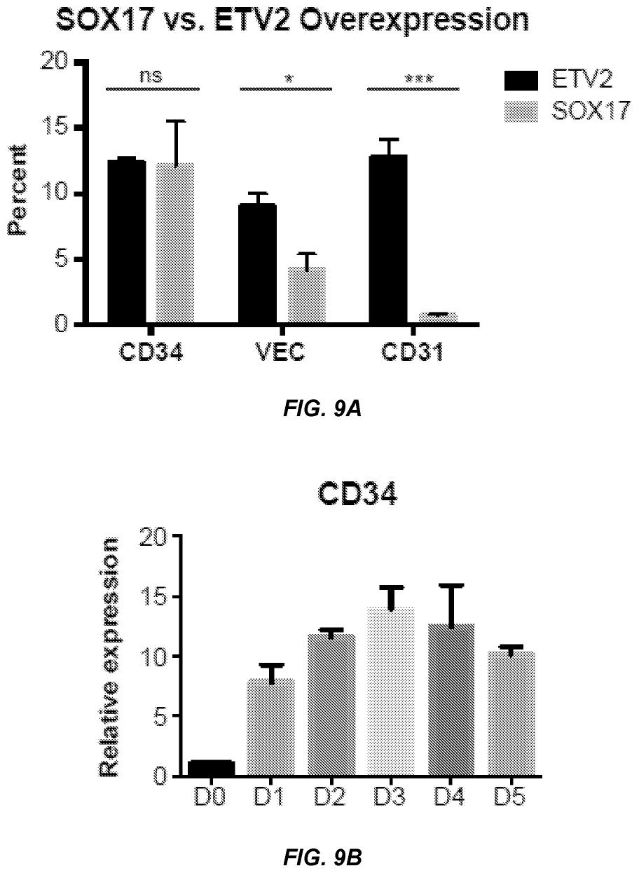

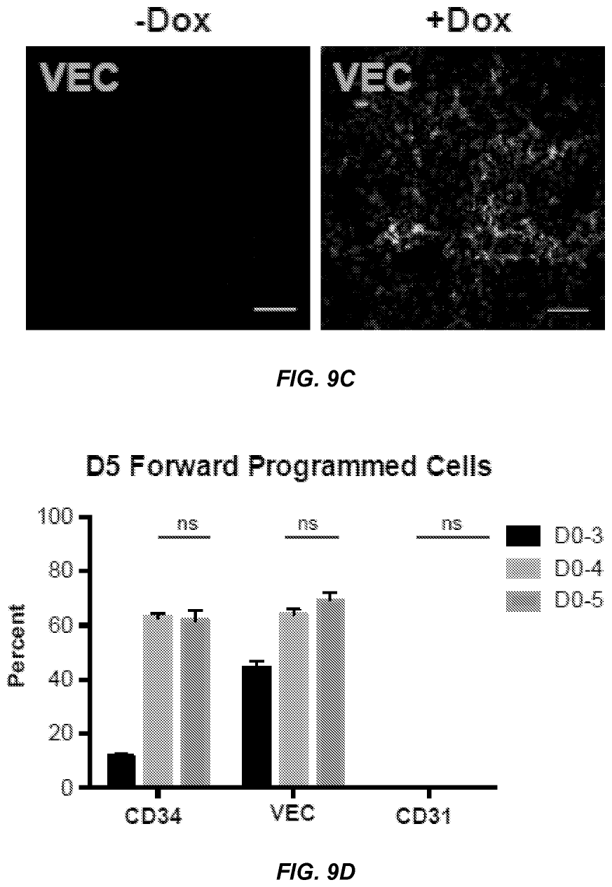

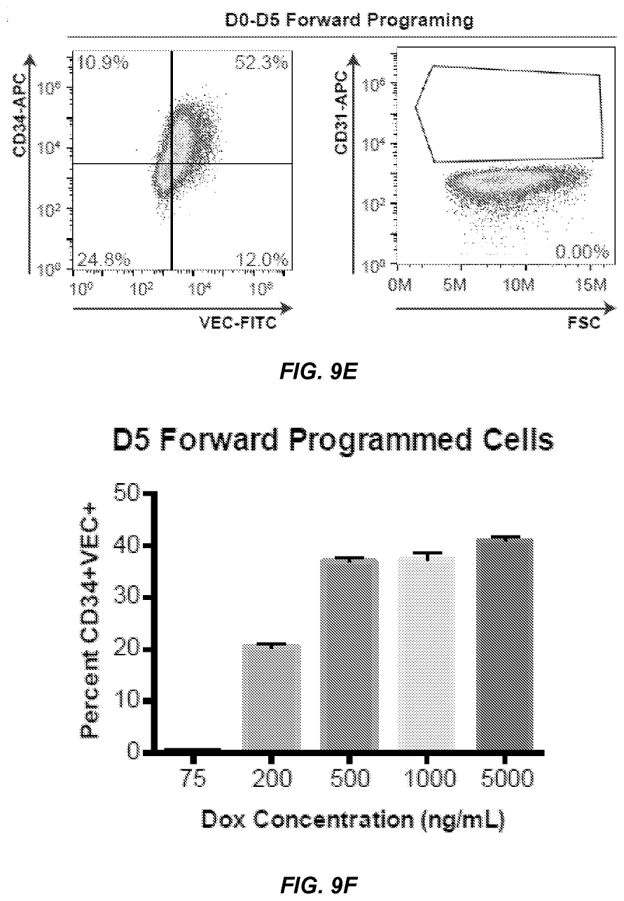

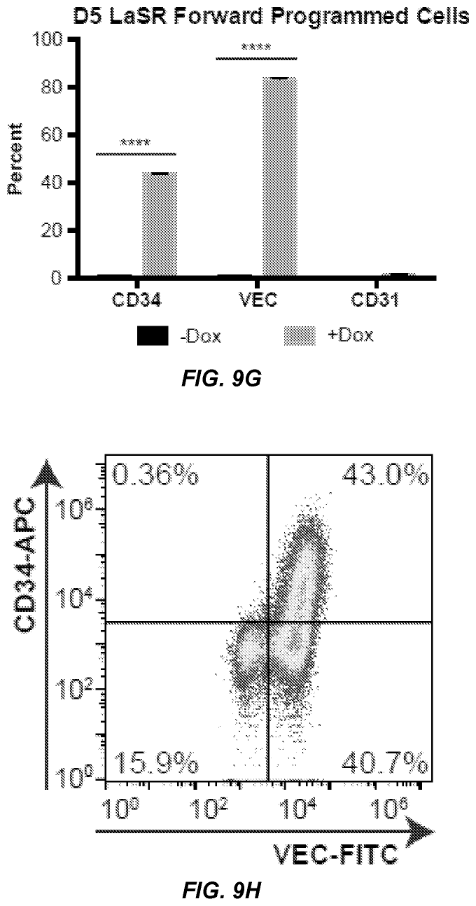

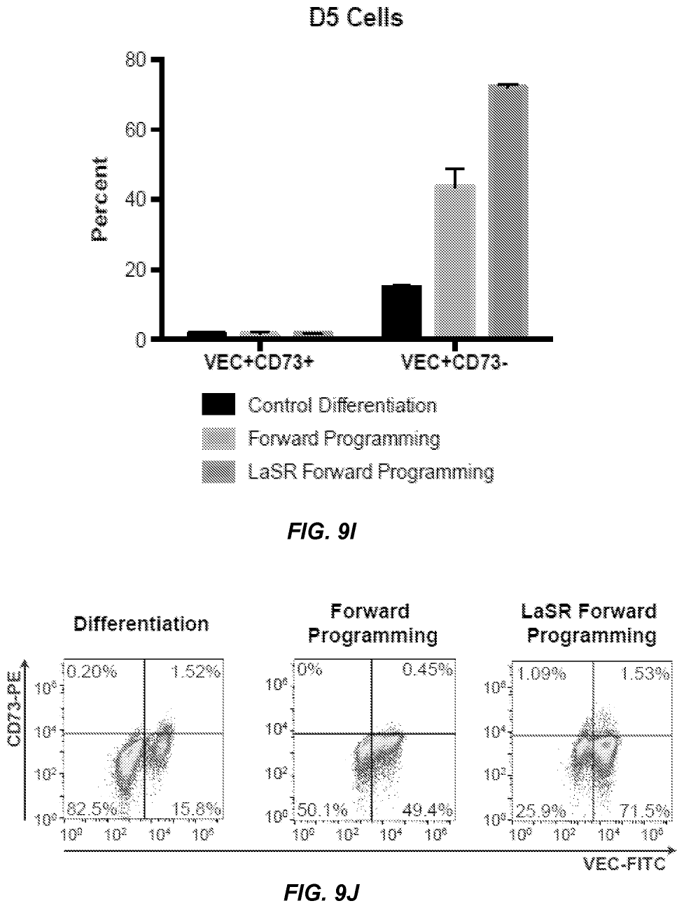

To evaluate to effects of SOX17 overexpression alone on the differentiation of hPSCs into HE cells, transgenic XLone-SOX17 hPSCs were treated with or without Dox in a basal medium (FIG. 8). A loss of pluripotency was observed by a notable loss of stem cell morphology as early as day 1 (FIG. 8). Day 5 cells cultured in basal media without Dox did not turn on the expression of CD34, CD31, or VEC; however, when Dox treatment is applied, a population of the cells show expression of CD34 and VEC, with CD34 RNA expression increasing as early as day 1 (FIGS. 9A-C and 5).

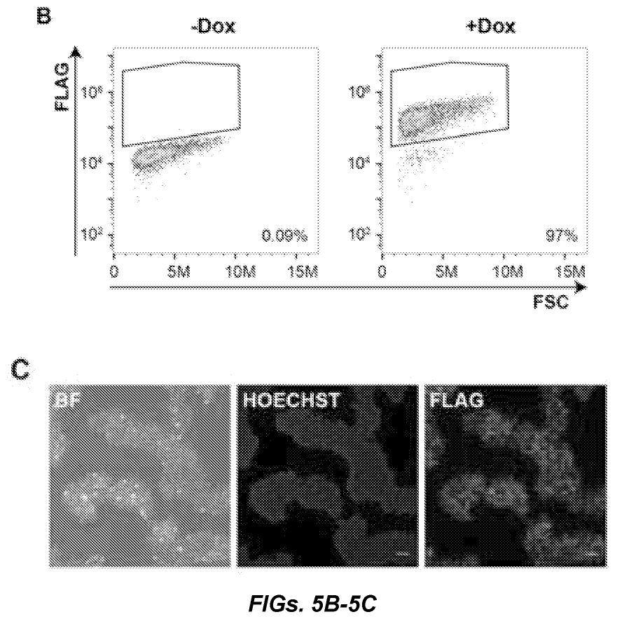

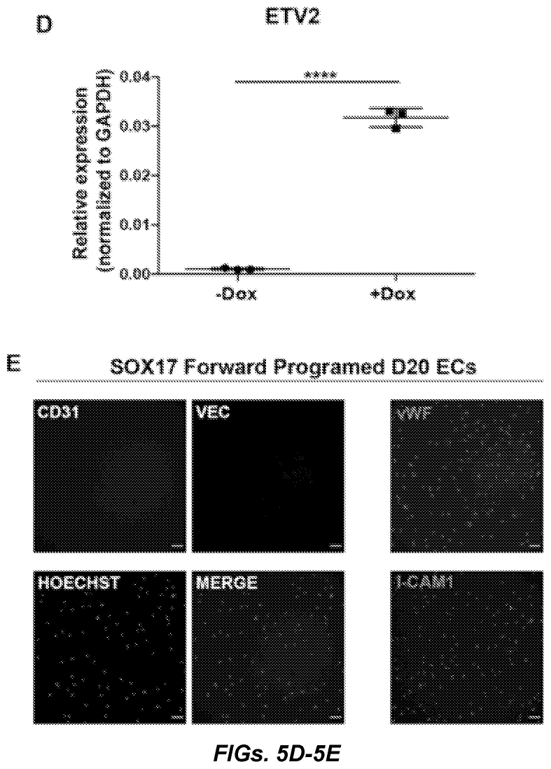

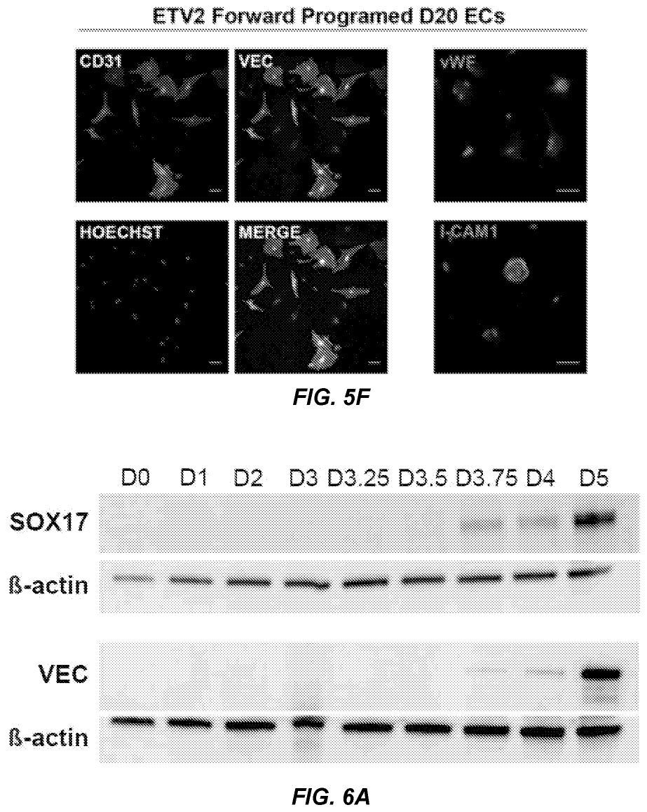

In view of the fact that other TFs such as ETV2 have been used to forward program hPSCs to endothelial progenitors (I. Elcheva, et al., “Direct induction of haematoendothelial programs in human pluripotent stem cells by transcriptional regulators,” Nat. Commun. 5:1-11 (2014), which is hereby incorporated by reference in its entirety), marker expression for the cells resulting from ETV2 and SOX17 forward programming were compared. The same XLone plasmid construct was used to establish an H9+XLone-ETV2 cell line (FIGS. 5A-D). These cells were then forward programmed in basal media with or without dox and analyzed on day 5. Compared to SOX17 forward programmed cells, there was no significant difference in CD34 (p=0.95); however, there was a significant increase in VEC (p=0.037) and CD31 (p=0.0008) expression with ETV2 (FIG. 9A). The difference in marker profile could be indicative of a difference in progenitor potential. To compare further endothelial cell potential, the day 5 cells were further differentiated to day 20 using commercial endothelial cell media. After 20 days, the ETV2 forward programmed progenitors resulted in cells that strongly expressed mature endothelial markers CD31, VEC, vWF, and I-CAM1 (FIG. 5F). These markers were not detected in SOX17 forward programmed cells, indicating these cells did not further differentiate into endothelial cells via endothelial cell media treatment (FIG. 5E).

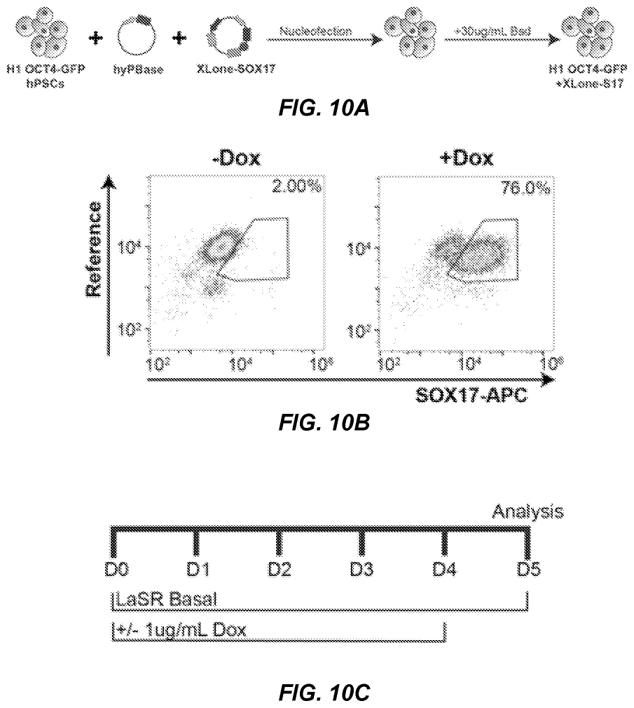

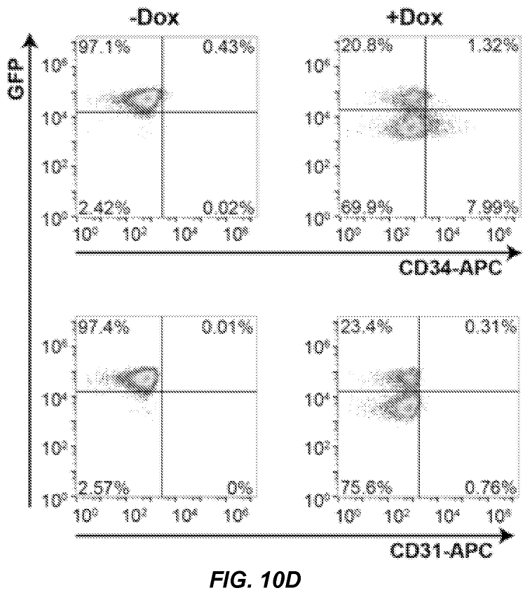

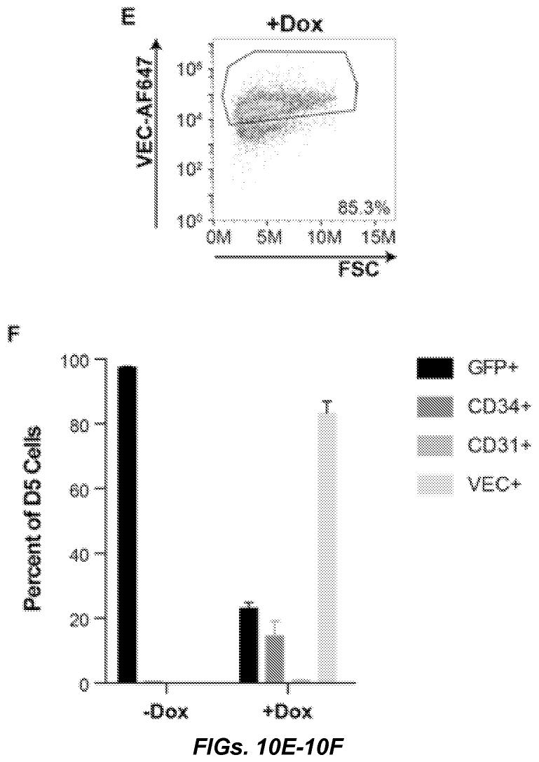

To confirm that SOX17-mediated forward programming was not cell line dependent, XLone-SOX17 cell lines were generated and validated using H1 OCT4-GFP reporter cells and 6-9-9 iPSCs (FIGS. 10A-B). Cells were then cultured with or without Dox treatment in basal media (FIG. 10C). The loss of pluripotency was confirmed by decrease in the GFP expression for H1 OCT4-GFP+XLone-SOX17 cells from 97.1% on day 0 to 20.8% on day 5 (FIG. 10D). The percentage of the cells expressing CD34 and VEC varied by cell line, but CD34 and VEC expression were obtained with overexpression of SOX17 alone across multiple hPSC lines (FIGS. 10D-F).

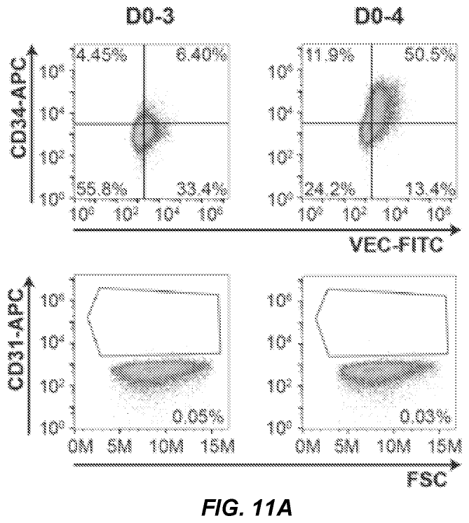

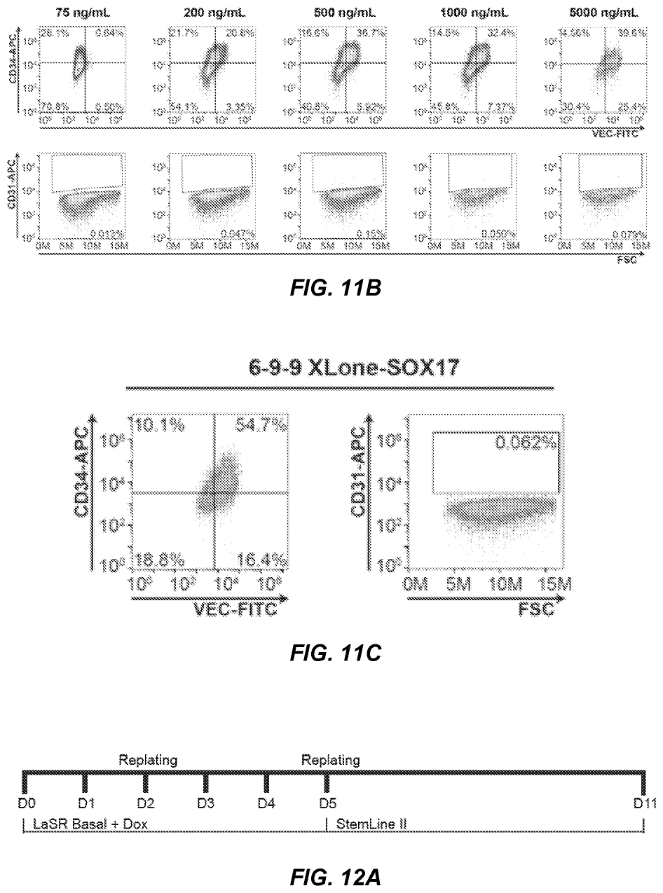

To further increase the efficiency of SOX17 forward programming, it was assumed that duration of Dox treatment and/or passaging cells during differentiation may play a role in increasing efficiency. hPSCs were forward programmed, passaged on day 2, and the duration of Dox treatment was varied from 3 to 5 days (FIG. 9D). With three days of Dox treatment, the CD34⁺ population was found similar to that of forward programming without a day 2 passage (FIGS. 9A, 9D, and 11A). However, the size of the VEC⁺ population increased from less than 5% to 44.2%±2.8% (FIGS. 9A, 9D, and 11A). Longer duration of Dox treatment increased the efficiency to at least 51%±2.3% CD34⁺VEC⁺ with no significant difference between 4 or 5 days of Dox treatment (CD34 p=0.79, VEC p=0.23) (FIGS. 9D-E and 11A). This demonstrated that longer Dox treatment and passaging on day 2 increased the efficiency of SOX17 forward programming. The passage could serve as selection for cells that are being forward programmed or the change in cell-cell contact could further enhance differentiation.

After optimizing the required duration of Dox treatment, a range of concentrations was tested to determine the optimal level of transgene activation (L. N. Randolph, et al., “An all-in-one, Tet-On 3G inducible PiggyBac system for human pluripotent stem cells and derivatives,” Sci. Rep. 7:1549 (2017), which is hereby incorporated by reference in its entirety). It was found that lower Dox concentrations resulted in a decreased CD34+VEC+ population and that at least 500 ng/mL Dox was required to achieve maximal efficiency (FIGS. 9F and 11B). This indicates that maximal transgene expression is required for SOX17 forward programming. This finding was replicated with 6-9-9+XLone-SOX17 cells forward programmed using the optimized conditions and obtained 57.2% 2.8% of the resulting cells expressing CD34 and VEC (FIG. 11C).

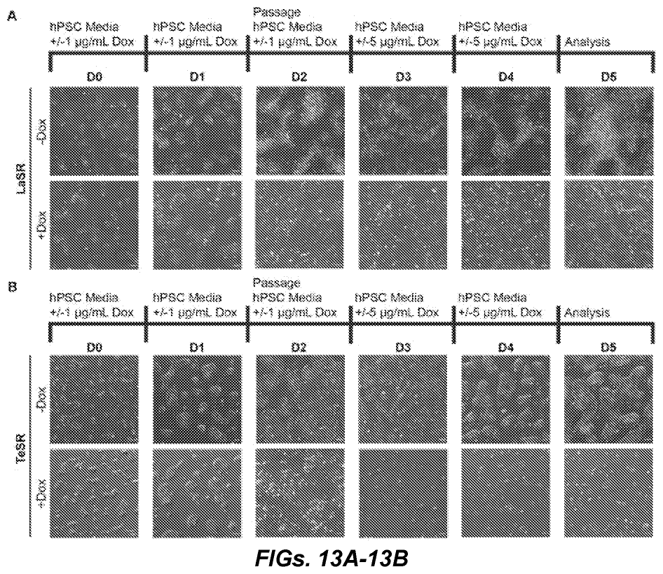

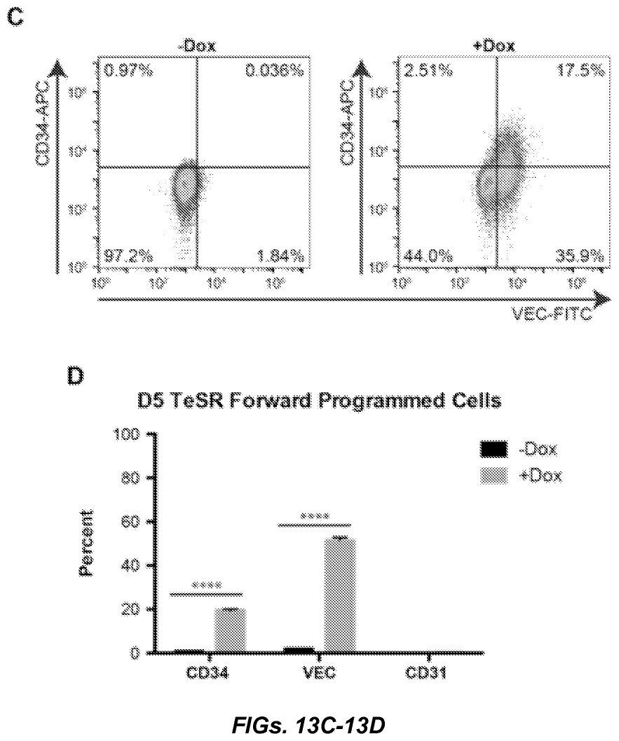

In an effort to examine the potency of SOX17 as a mediator of forward programming, the effects of SOX17 forward programming were tested in hPSCs cultured in hPSC media. Cells were cultured in one of two hPSC media, LaSR or mTeSR1, in the presence or absence of Dox (FIGS. 13A-B). SOX17 forward programming was able to overcome the pluripotency maintenance signals in hPSC media and cause cells to differentiate as evidenced by loss of pluripotent morphology (FIGS. 13A-B). Day 5 flow cytometry analysis showed the presence of CD34+VEC+ cells and CD34−VEC+ cells (FIGS. 9G-H and 13C-D). The expression of CD31 in the day 5 cells was not observed, similar to the observations made in basal media (FIGS. 9G and 13D). Both hPSC media produced CD34+VEC+ cells and CD34−VEC+ cells, albeit at different efficiencies (FIGS. 9H and 13C). This could be due to the increased amount of bovine serum albumin in mTeSR1 as compared to LaSR media; nevertheless, both media produced statistically significant populations of differentiated cells expressing CD34 (LaSR p=5.75e-8, mTeSR1 p=2.88e-6) and VEC (LaSR p=6.78e-9, mTeSR1 p=1.18e-6) (FIGS. 9G and 13D). Collectively, these results demonstrated SOX17 forward programming potency can overcome the presence of pluripotency growth factors in hPSC media to efficiently produce CD34+VEC+ cells.

HE differentiations often result in heterogeneous populations of cells, as is common with many in vitro methods (K.-D. Choi, et al., “Identification of the hemogenic endothelial progenitor and its direct precursor in human pluripotent stem cell differentiation cultures,” Cell Rep. 2:553-567 (2012); A. Ditadi, et al., “Human definitive haemogenic endothelium and arterial vascular endothelium represent distinct lineages,” Nat. Cell Biol. 17:580-91 (2015); C. M. Sturgeon, et al., “Wnt signaling controls the specification of definitive and primitive hematopoiesis from human pluripotent stem cells,” Nat. Biotechnol. 32:554-561 (2014), which are hereby incorporated by reference in their entirety). To further characterize the cells obtained from SOX17 forward programming, the expression of an additional marker, CD73, which is not expressed in HE populations but is expressed in other non-HE endothelial cells (K.-D. Choi, et al., “Identification of the hemogenic endothelial progenitor and its direct precursor in human pluripotent stem cell differentiation cultures,” Cell Rep. 2:553-567 (2012), which is hereby incorporated by reference in its entirety) was assessed. The day 5 cells resulting from CH-induced differentiation, forward programming in basal media, and forward programming in hPSC media were compared and it was found that none of these methods produced more than an average of 1.62% of the cells expressing both VEC and CD73. This indicates that forward programming does not result in contamination with this previously identified non-HE population (FIGS. 9I-J).

### Example 5—SOX17 Forward Programming is Dependent on β-Catenin

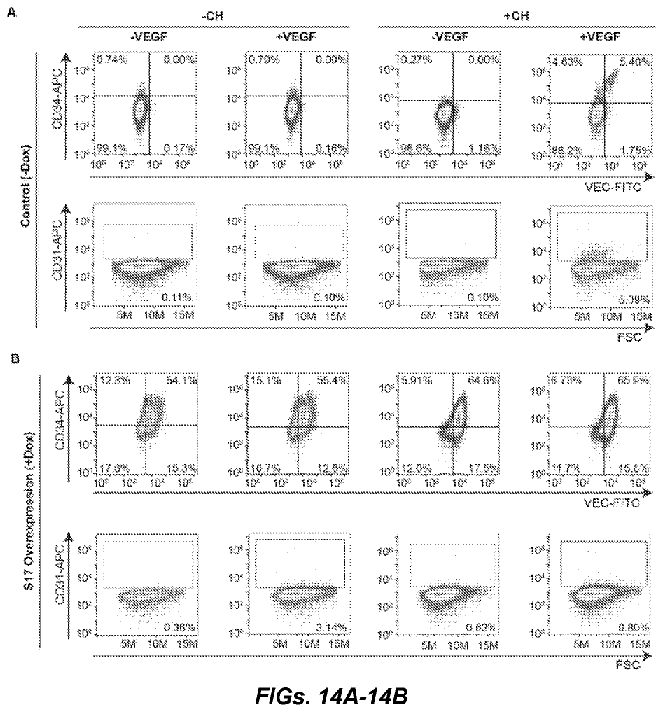

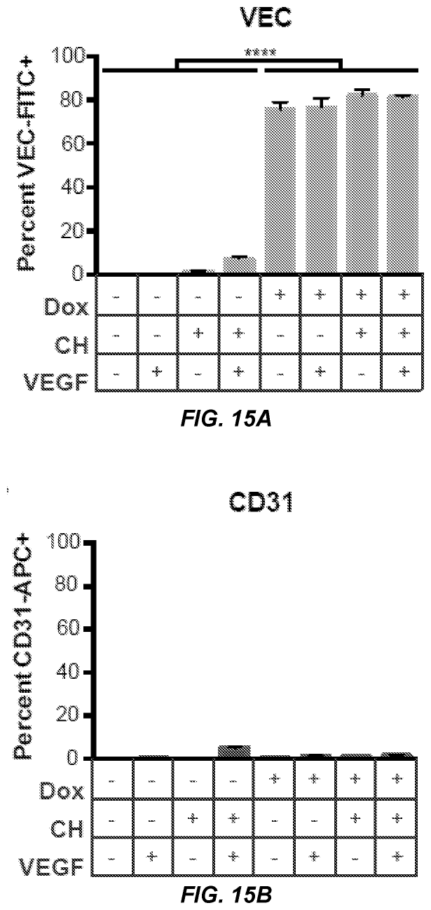

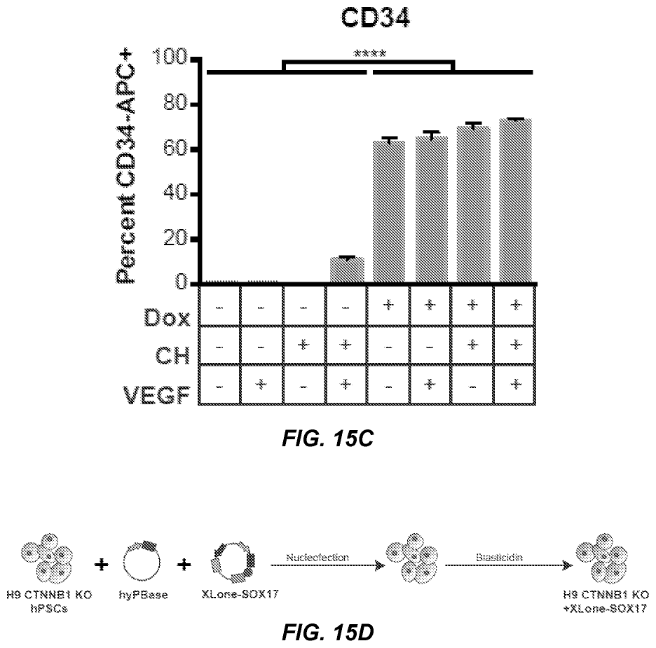

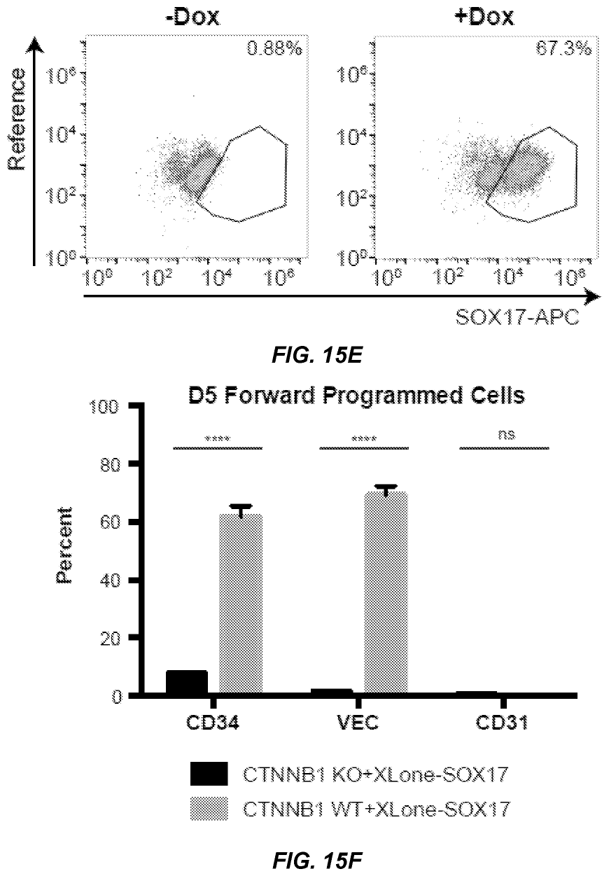

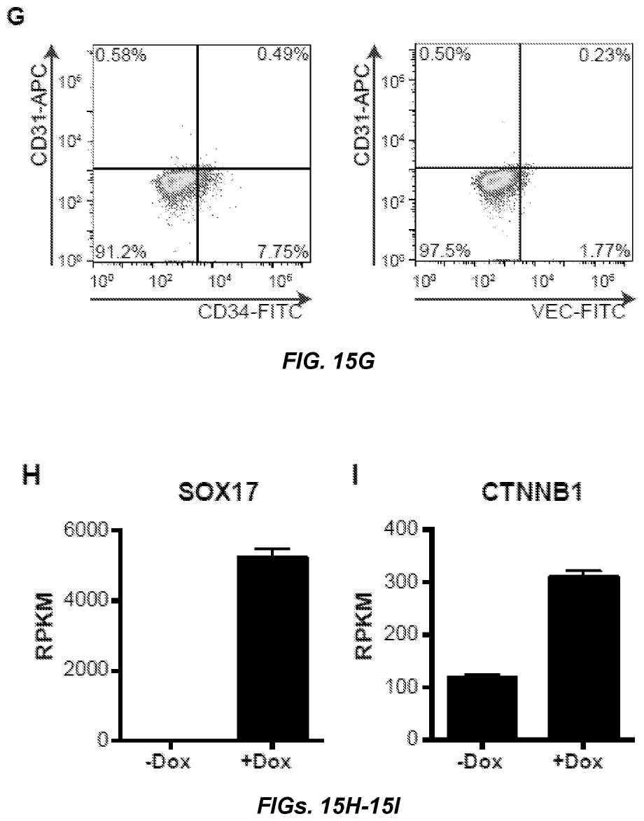

To study interaction between SOX17 forward programming and CH-induced differentiation, H9+XLone-SOX17 cells to day 5 were differentiated with and without Dox while varying the presence of CH and VEGF. For conditions treated without Dox, cells that received no Wnt activation (CH) showed none of the HE markers (CD34, VEC, and CD31) (FIGS. 15A-C and 14A). Cells differentiated using only Wnt activation showed minimal CD34 CD31, or VEC (FIGS. 15A-C and 14A). This is supported by previous findings showing that female cell lines require the addition of VEGF to differentiate to endothelial progenitors (L. N. Randolph, et al., “Sex-dependent VEGF expression underlies variations in human pluripotent stem cell to endothelial progenitor differentiation,” Sci. Rep. 9:1-8 (2019), which is hereby incorporated by reference in its entirety). Cells differentiated with Wnt activation and VEGF expressed CD31, CD34, and VEC (FIGS. 15A-C and 14A). For conditions treated with Dox, CD34 expression increased from at most 11%±1.01% without Dox treatment to 67.6%±1.50% with Dox treatment (FIGS. 15B-C and 14B). The VEC⁺ population increased with Dox treatment similar to the observations for CD34 (FIGS. 15A and 14B). The addition of CH in the presence of SOX17 overexpression did not significantly affect the percentage of the CD34+VEC+ population.

Wnt/β-catenin signaling plays critical roles in mesoderm and HE specification from hPSCs (3, 5, 19, 28). Because CH addition did not further increase efficiency of SOX17 forward programming, whether Wnt/β-catenin signaling is required for SOX17 forward programming was investigated. To do this, the efficacy of forward programming in the absence of β-catenin was examined. The H9 CTNNB1 KO cells (X. Lian, et al., “Interrogating Canonical Wnt Signaling Pathway in Human Pluripotent Stem Cell Fate Decisions Using CRISPR-Cas9,” Cell. Mol. Bioeng. 9:325-334 (2016), which is hereby incorporated by reference in its entirety) were used, the XLone-SOX17 construct was introduced, and inducible cell line function was verified (FIGS. 15D-E). These cells were then forward programmed with Dox in basal media for five days. The resulting day 5 cells showed a statistically significant decrease in the expression of CD34 (p=5.52e-5) and VEC (p=1.26e-5) as compared to cells with β-catenin (FIGS. 15F-G). To examine whether CTNNB1 expression is upregulated when SOX17 is overexpressed, RNA-seq data of Dox inducible SOX17 hPSCs was analyzed with and without SOX17 overexpression (Y. Nakatake, et al., “Generation and Profiling of 2,135 Human ESC Lines for the Systematic Analyses of Cell States Perturbed by Inducing Single Transcription Factors,” Cell Rep. 31:107655 (2020), which is hereby incorporated by reference in its entirety). It was confirmed that SOX17 was robustly overexpressed when treated with Dox and found that CTNNB1 was also upregulated as a result (FIGS. 15H-I). This illustrated that β-catenin is required in SOX17 forward programming.

### Example 6—Further Differentiated SOX17 Forward Programmed Cells Strongly Upregulate Hematopoietic Transcription Factors and Yields Cells Capable of Multi-Lineage Hematopoiesis

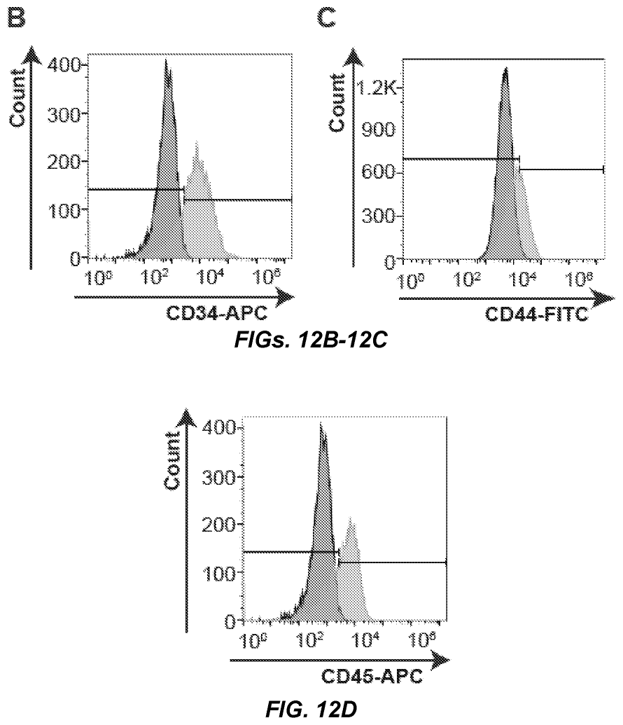

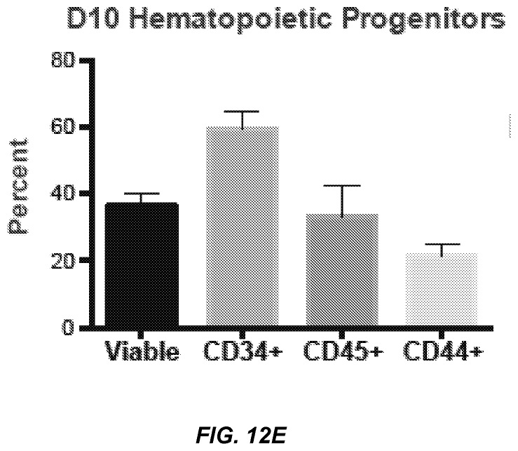

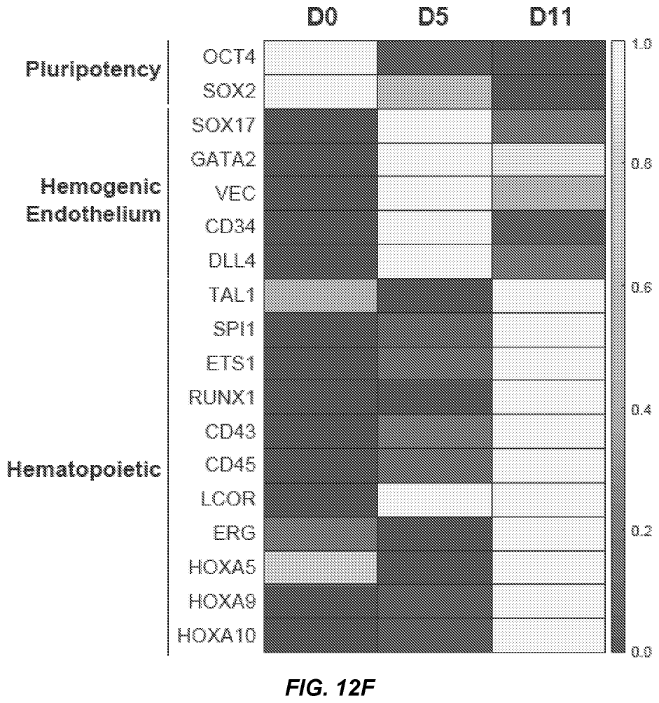

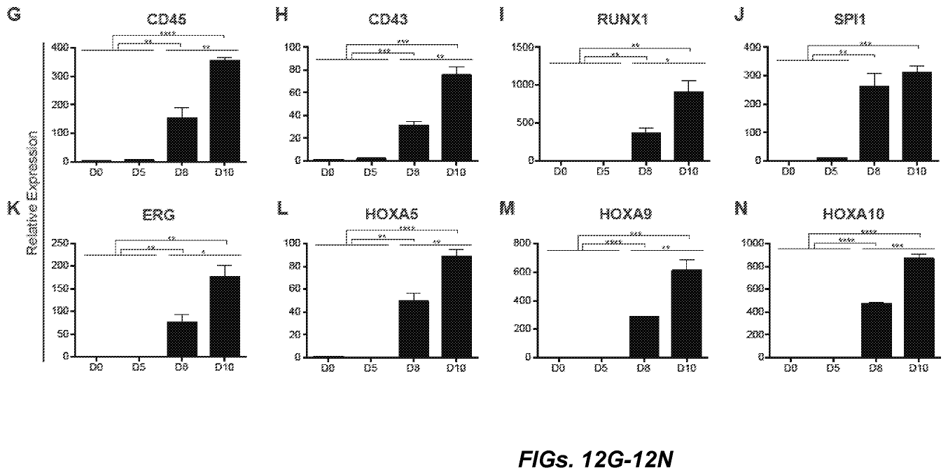

Based on the results showing SOX17 plays a critical role in HE progenitor acquisition, the extent to which SOX17 forward programming activates downstream hematopoietic gene programs was studied. The cells were forward programmed to day 5 via SOX17 overexpression and then Dox was removed and the cells were cultured in StemLine II hematopoietic stem cell expansion media (FIG. 12A). By day 10, many floating cells were observed and collected to assess hematopoietic marker expression and viability. 36.6%±3.5% of the suspension cells were found viable and there was notable expression of CD34, CD45, and CD44 (FIGS. 12B-E). The floating cells on days 8, 10 and 11 were collected for qPCR analysis.

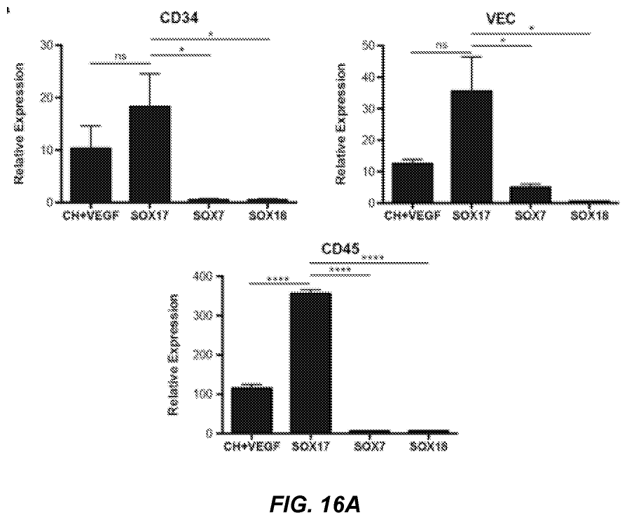

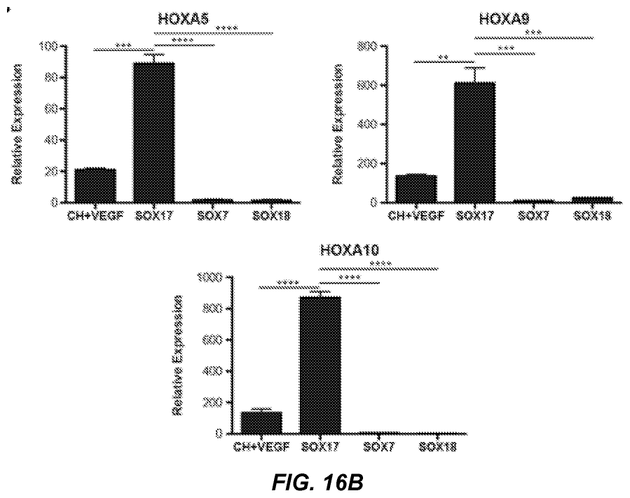

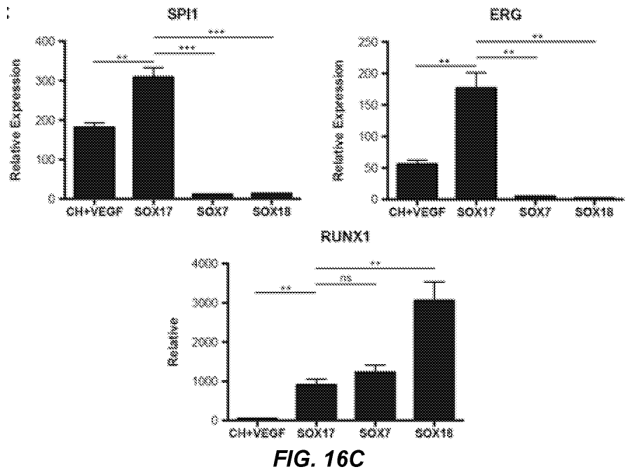

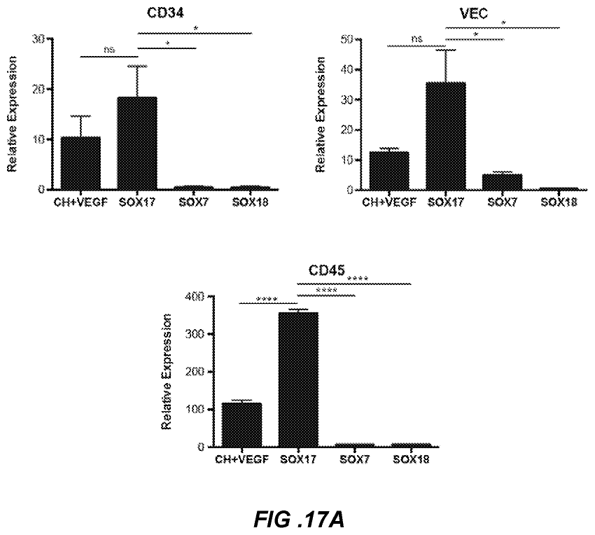

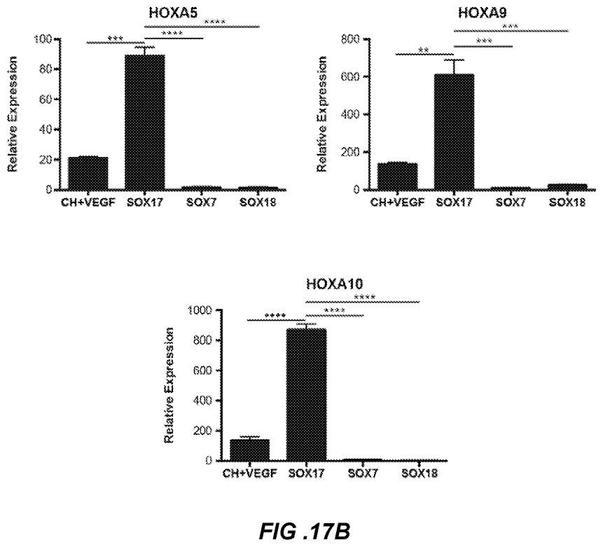

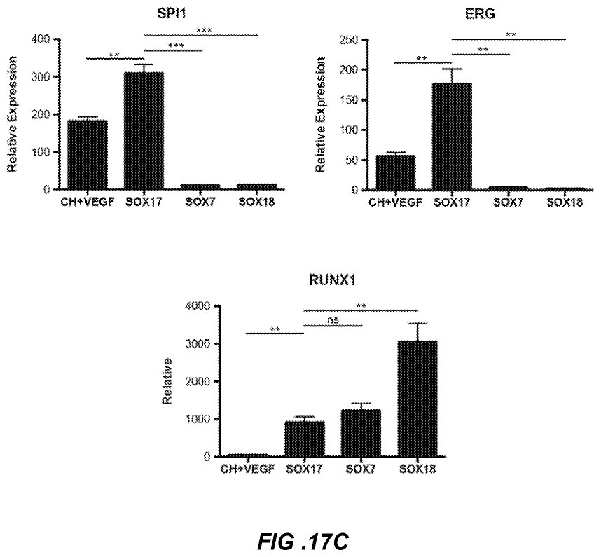

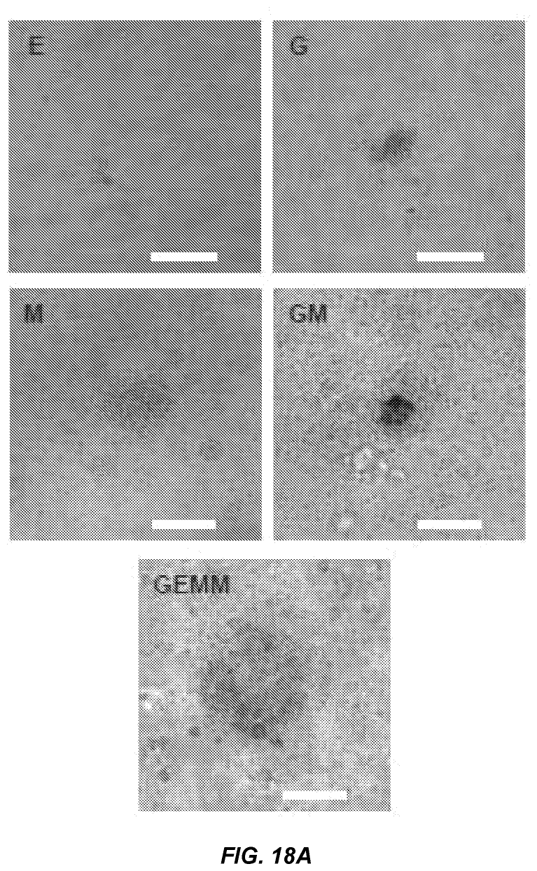

qPCR analysis was performed for genes associated with hPSCs, HE cells, and hematopoietic progenitors, including TFs previously used for hematopoietic reprogramming or forward programming. Stage specific peak expression for pluripotency genes (OCT4 and SOX2) on day 0, HIE genes (SOX17, GATA2, CD31, VEC, CD34, and DLL4) on day 5, and hematopoietic genes (TAL1, SPI1, ETS1, RUNX1, CD43, CD45, LCOR, ERG, HOXA5, HOXA9, HOXA10) on day 11 (FIG. 12F) were observed. Further examination showed significant upregulation of hematopoietic surface markers CD45 and CD43 on both days 8 and 10, with 355-fold (p=4.03e-6) and 75-fold (p=0.0004) increases in relative expression respectively on day 10 (FIGS. 12G-H). Furthermore, there was a significantly more CD45 expression in SOX17 forward programmed cells on day 10 as compared to control differentiated cells (p=6.97e-5), SOX7 forward programmed cells (p=4.28e-6), and SOX18 forward programmed cells (p=4.31e-6) (FIG. 16A).

RUNX1 is a TF that plays a well-established role in hematopoietic fate acquisition and has been used for forward hematopoietic programming (T. Okuda, et al., “AML1, the target of multiple chromosomal translocations in human leukemia, is essential for normal fetal liver hematopoiesis,” Cell 84:321-330 (1996); T. North, et al., “Cbfa2 is required for the formation of intra-aortic hematopoietic clusters,” Development 126:2563-2575 (1999); R. Sugimura, et al., “Haematopoietic stem and progenitor cells from human pluripotent stem cells,” Nature 545:432-438 (2017), which are hereby incorporated by reference in their entirety). A significant increase in RUNX1 on days 8 (p=0.006) and 10 (p=0.003) was detected with expression approaching a 1000-fold increase over the day 0 expression level (FIG. 12I). Additionally, strong upregulation of TFs SPI1, ERG, HOXA5, HOXA9, and HOXA10 on days 8 and 10 was observed, with significant more expression than differentiated cells, and SOX7 or SOX18 forward programmed cells (FIGS. 12J-N and 16B-C). These factors along with RUNX1 have been used to forward program HE cells to a definitive hematopoietic fate (R. Sugimura, et al., “Haematopoietic stem and progenitor cells from human pluripotent stem cells,” Nature 545:432-438 (2017), which is hereby incorporated by reference in its entirety). Taken together, these data demonstrate SOX17 forward programming is sufficient for robust downstream activation of hematopoietic gene and TF networks.

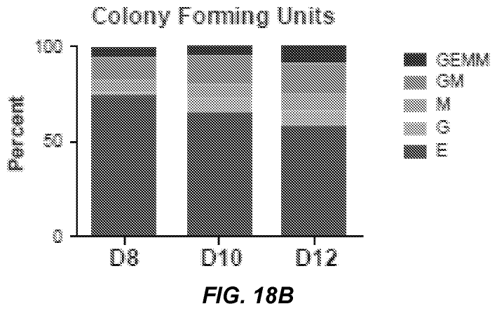

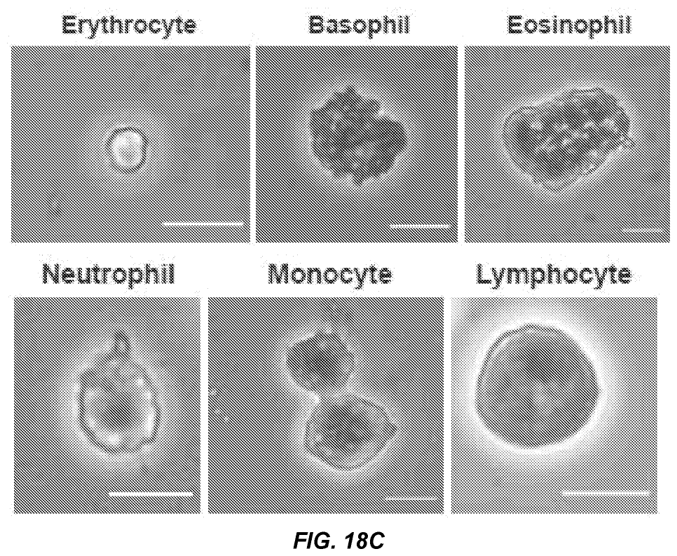

To test the functional hematopoietic capacity of our SOX17 forward programmed hematopoietic progenitors, a colony forming unit assay was performed using floating cells obtained on days 8, 10, and 12. All 5 types of colonies were observed with the majority being erythrocytic (FIGS. 18A-B). The presence of GM and GEMM colonies indicates that some of our progenitors are multipotent. A Giemsa stain on our day 10 cells was also performed to evaluate the types of cells already present after forward programming. Cells from all major blood lineages were identified including cells with monocyte and lymphocyte morphology (FIG. 18C). This demonstrates the ability of our cells to differentiate to multiple terminal hematopoietic lineages.

### Example 7—Discussion of Examples 1-6

Forward programming has proven to be an effective strategy for the generation of difficult to obtain cell types (R. Sugimura, et al., “Haematopoietic stem and progenitor cells from human pluripotent stem cells,” Nature 545:432-438 (2017), which is hereby incorporated by reference in its entirety). Herein, the first evidence showing that overexpression of SOX17 alone in hPSCs is sufficient for the generation of CD34+VEC+CD73− HE cells and floating CD34⁺ hematopoietic progenitors via an EHT in the absence of small molecules and growth factor has been provided. Furthermore, it has been demonstrated that the resulting hematopoietic progenitors have significantly upregulated key hematopoietic TFs and can further differentiate to multiple hematopoietic lineages.

Single cell RNA sequencing of the published endothelial progenitor differentiation confirmed the expression of HE markers and revealed differential expression of SOXF factors. When these factors were overexpressed in tandem with differentiation, however, only SOX17 increased differentiation efficiency as measured by the generation of CD34+VEC+ cells. VEC expression was not observed with all three SOXF factors. This is consistent with reports that SOX17 can bind to the VEC promotor in human cells, and SOX7 binds to the VEC promotor and activates VEC expression in murine cells (Y. Nakajima-Takagi, et al., Role of SOX17 in hematopoietic development from human embryonic stem cells,” Blood 121:447-458 (2013); G. Costa, et al., “SOX7 regulates the expression of VE-cadherin in the haemogenic endothelium at the onset of haematopoietic development,” Development 139: 1587-1598 (2012), which are hereby incorporated by reference in their entirety). Given the high degree of conservation and the similarities in SOXF subgroup binding sites, it is likely all three factors will have a similar effect on the VEC transcription in human cells. This is supported by the findings indicating that SOX17 expression occurs just prior to VEC emergence. CD31 expression was not detected with SOX17 forward programming; however due to the lack of HE specific markers that are not shared by other tissues and rarity of human HE cells during development it is difficult to determine definitively the marker profile of these cells. While the lack of CD31 expression was a departure from the marker profile typically observed with the established CH-induced differentiation, other studies have characterized HE populations without the use of CD31 (C. M. Sturgeon, et al., “Wnt signaling controls the specification of definitive and primitive hematopoiesis from human pluripotent stem cells,” Nat. Biotechnol. 32:554-561 (2014); G. I. Uenishi, et al., “NOTCH signaling specifies arterial-type definitive hemogenic endothelium from human pluripotent stem cells,” Nat. Commun. 9:1-14 (2018), which are hereby incorporated by reference in their entirety). Furthermore, early progenitor populations have been identified that express high levels of CD34 and VEC and no CD31 (“J. Patel, et al., Functional Definition of Progenitors Versus Mature Endothelial Cells Reveals Key SoxF-Dependent Differentiation Process,” Circulation 135:786-805 (2017), which is hereby incorporated by reference in its entirety). It is also possible SOX17 forward programming increases differentiation efficiency by producing a different endothelial progenitor lacking CD31 expression as is indicated by the comparison with ETV2 forward programming.

To better understand the functional significance of SOX17, loss of function studies using Cas13d were performed and SOX17 knockdown was found to significantly impede the ability of hPSCs to differentiate to HE cells. This is consistent with previous findings in murine and human systems (R. L. Clarke, et al., “The expression of Sox17 identifies and regulates haemogenic endothelium,” Nat. Cell Biol. 15:502-10 (2013); Y. Nakajima-Takagi, et al., “Role of SOX17 in hematopoietic development from human embryonic stem cells,” Blood 121:447-458 (2013); K. Kim, et al., “SoxF Transcription Factors Are Positive Feedback Regulators of VEGF Signaling,” Circ. Res. 119:839-852 (2016), which are hereby incorporated by reference in their entirety). Having confirmed functional importance of SOX17 by loss of function, forward programming by SOX17 overexpression was shown to be sufficient to obtain CD34+VEC+ CD73− cells from multiple hPSC lines in a variety of different culture media, including hPSC media. Forward programming conditions were optimized to find the ideal temporal window and level for transgene expression. Furthermore, SOX17 forward programming was found to require p-catenin indicating dependence on Wnt/p-catenin signaling, a hallmark of HE development (C. M. Sturgeon, et al., “Wnt signaling controls the specification of definitive and primitive hematopoiesis from human pluripotent stem cells,” Nat. Biotechnol. 32:554-561 (2014), which is hereby incorporated by reference in its entirety). This is the first report to show that SOX17 overexpression alone can efficiently generate HE cells.

These data demonstrate that overexpression of SOX17 during the first five days was sufficient to propel the cells through an EHT resulting in robust generation of viable CD34+ hematopoietic progenitors. Analysis of the expression of stage specific markers and TFs showed developmental progression from HE cells to hematopoietic progenitors. SOX17 forward programmed cells were also able to strongly upregulate TFs associated with definitive hematopoietic fate and commitment. HOXA genes such as HOXA5, HOXA9, and HOXA10 were significantly upregulated in hematopoietic progenitors that arose from SOX17 forward programmed cells. This is consistent with the understanding that HOXA genes are only turned on by early definitive hematopoietic fate specification (A. Ivanovs, et al., “Human haematopoietic stem cell development: From the embryo to the dish,” Development 144:2323-2337 (2017); E. S. Ng, et al., Differentiation of human embryonic stem cells to HOXA+ hemogenic vasculature that resembles the aorta-gonad-mesonephros,” Nat. Biotechnol. 34:1168-1179 (2016), which are hereby incorporated by reference in their entirety). Furthermore, HOXA genes are selectively expressed in human fetal liver and umbilical cord blood derived hematopoietic progenitors (E. S. Ng, et al., Differentiation of human embryonic stem cells to HOXA+ hemogenic vasculature that resembles the aorta-gonad-mesonephros,” Nat. Biotechnol. 34:1168-1179 (2016), which is hereby incorporated by reference in its entirety).

Moreover, SOX17 forward programming led to significant increases in expression for all of the TFs used by Sugimura et al. to forward program HE cells to definitive hematopoietic stem and progenitor cells (R. Sugimura, et al., “Haematopoietic stem and progenitor cells from human pluripotent stem cells,” Nature 545:432-438 (2017), which is hereby incorporated by reference in its entirety). This included marked upregulation of RUNX1, which is essential for definitive hematopoietic development (A. Ditadi, et al., “Human definitive haemogenic endothelium and arterial vascular endothelium represent distinct lineages,” Nat. Cell Biol. 17:580-91 (2015), E. S. Ng, et al., Differentiation of human embryonic stem cells to HOXA+ hemogenic vasculature that resembles the aorta-gonad-mesonephros,” Nat. Biotechnol. 34:1168-1179 (2016); T. North, et al., “Cbfa2 is required for the formation of intra-aortic hematopoietic clusters,” Development 126:2563-2575 (1999), which are hereby incorporated by reference in their entirety). Additional analysis of cell fate potential by further differentiation revealed the multilineage potential of our hematopoietic progenitors. It has been demonstrated that SOX17 forward programming results in an EHT and activation of hematopoietic gene networks.

In summary, this is the first report of forward programming of hPSCs to HE cells with SOX17. This is also the first evidence of a single TF mediating an EHT with human cells in the absence of small molecules and growth factors. Furthermore, these findings place SOX17 in a place of heightened importance in human hematopoietic development and demand further investigation. For example, additional study of triphasic SOX17 expression control, mimicking existing evidence from murine development, could lead to further improvements for in vitro forward programming and new insights for human HSC emergence (I. Kim, et al., “Sox17 Dependence Distinguishes the Transcriptional Regulation of Fetal from Adult Hematopoietic Stem Cells,” Cell 130:470-483 (2007), which is hereby incorporated by reference in its entirety). These findings are expected to increase our understanding of human hematopoietic development and lead to improved manufacturing of therapeutically relevant cells.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of producing an enriched preparation of hemogenic endothelial progenitor cells, said method comprising:
providing a population of pluripotent stem cells;
inducing expression of a SOXF transcription factor in pluripotent stem cells of the population; and
culturing the population of pluripotent stem cells expressing the SOXF transcription factor, whereby the enriched preparation of hemogenic endothelial progenitor cells is produced as a result of said culturing.

2. The method of claim 1, wherein the hemogenic endothelial progenitor cells of the enriched preparation co-express VE-cadherin (VEC) and CD34.

3. The method of claim 1, wherein the hemogenic endothelial progenitor cells of the enriched preparation do not express CD31 or CD73.

4. The method of claim 1, wherein at least 60% of the cells in the enriched preparation are hemogenic endothelial progenitor cells co-expressing VEC and CD34.

5. The method of claim 1, wherein the population of pluripotent stem cells is a population of human embryonic stem cells.

6. The method of claim 1, wherein the population of pluripotent stem cells is a population of human induced pluripotent stem cells.

7. The method of claim 1, wherein the preparation of hemogenic endothelial progenitor cells is a preparation of human hemogenic endothelial progenitor cells.

8. The method of claim 1, wherein the SOXF transcription factor is selected from SOX-7, SOX-17, SOX-18, and any combination thereof.

9. The method of claim 1, wherein said inducing comprises:
introducing a nucleic acid molecule encoding the SOXF transcription factor into cells of the pluripotent stem cell population.

10. The method of claim 9, wherein the nucleic acid molecule encoding the SOXF transcription factor is a SOXF transcription factor RNA.

11. (canceled)

12. (canceled)

13. The method of claim 1, wherein the SOXF RNA is a human SOX17 RNA.

14. The method of claim 1, wherein the SOXF RNA is a human SOX7 RNA.

15. The method of claim 1, wherein the SOXF RNA is a human SOX18 RNA.

16. The method of claim 1, wherein the SOXF RNA is encapsulated in a delivery vehicle and said introducing comprises introducing said delivery vehicle into cells of the pluripotent stem cell population.

17. The method of claim 1, wherein the SOXF RNA is contained in an expression vector and said introducing comprises introducing said expression vector into cells of the pluripotent stem cell population.

18. The method of claim 17, wherein the SOXF transcription factor RNA of the expression vector is operatively coupled to an inducible promoter.

19. The method of claim 18, wherein the inducible promoter is a drug inducible promoter, and said inducing comprises:
administering, to the pluripotent stem cell population comprising the SOXF RNA expression vector, an effective amount of the drug capable of inducing promoter mediated SOXF transcription factor expression from the expression vector.

20. (canceled)

21. The method of claim 17 further comprising:
removing the expression vector from the hemogenic endothelial progenitor cells of the preparation after said culturing.

22. (canceled)

23. An enriched preparation of hemogenic endothelial progenitor cells produced via the method of claim 1.

24. (canceled)

25. (canceled)

26. A method of treating a subject having a condition mediated by a loss hematopoietic stem cells, said method comprising:
administering to the subject the enriched preparation of claim 23, or a preparation of cells differentiated from said enriched preparation under conditions effective to treat the condition.

27.-71. (canceled)


SEQUENCE LISTING (52 sequences)

SEQ ID NO: 1 - DNA, 1657 bp, Homo sapien
gccatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtgacaggtccctgatccgccccagcctgcaggccagaagcagtgttacacacttcctggaggagctaaggaaatcctcagactcctgggtttttgttgttgctgttgttgttttttaaaaggtgtgttggcatataatttatggtaatttattttgtctgccacttgaacagtttgggggggtgaggtttcatttaaaatttgttcagagatttgtttcccatagttggattgtcaaaaccctatttccaagttcaagttaactagctttgaatgtgtcccaaaacagcttcctccatttcctgaaagtttattgatcaaagaaatgttgtcctgggtgtgttttttcaatcttctaaaaaataaaatctggaatcctgcttttttgctctactagtacctctgtcacacta

SEQ ID NO: 2 - protein, 414 aa, Homo sapien [sequence omitted]

SEQ ID NO: 3 - protein, 1482 aa, Artificial
  Modified Sox 17 mRNA
ACANNNGCNNCNGACACAACNGNGNNCACNAGCAACCNCAAACAGACACCGGANCCANGGCANGCGAANNCGCNAGCAAGCNNGGNACCGCCACCANGAGCAGCCCGGANGCGGGANACGCCAGNGACGACCAGAGCCAGACCCAGAGCGCGCNGCCCGCGGNGANGGCCGGGCNGGGCCCCNGCCCCNGGGCCGAGNCGCNGAGCCCCANCGGGGACANGAAGGNGAAGGGCGAGGCGCCGGCGAACAGCGGAGCACCGGCCGGGGCCGCGGGCCGAGCCAAGGGCGAGNCCCGNANCCGGCGGCCGANGAACGCNNNCANGGNGNGGGCNAAGGACGAGCGCAAGCGGCNGGCGCAGCAGAANCCAGACCNGCACAACGCCGAGNNGAGCAAGANGCNGGGCAAGNCGNGGAAGGCGCNGACGCNGGCGGAGAAGCGGCCCNNCGNGGAGGAGGCAGAGCGGCNGCGCGNGCAGCACANGCAGGACCACCCCAACNACAAGNACCGGCCGCGGCGGCGCAAGCAGGNGAAGCGGCNGAAGCGGGNGGAGGGCGGCNNCCNGCACGGCCNGGCNGAGCCGCAGGCGGCCGCGCNGGGCCCCGAGGGCGGCCGCGNGGCCANGGACGGCCNGGGCCNCCAGNNCCCCGAGCAGGGCNNCCCCGCCGGCCCGCCGCNGCNGCCNCCGCACANGGGCGGCCACNACCGCGACNGCCAGAGNCNGGGCGCGCCNCCGCNCGACGGCNACCCGNNGCCCACGCCCGACACGNCCCCGCNGGACGGCGNGGACCCCGACCCGGCNNNCNNCGCCGCCCCGANGCCCGGGGACNGCCCGGCGGCCGGCACCNACAGCNACGCGCAGGNCNCGGACNACGCNGGCCCCCCGGAGCCNCCCGCCGGNCCCANGCACCCCCGACNCGGCCCAGAGCCCGCGGGNCCCNCGANNCCGGGCCNCCNGGCGCCACCCAGCGCCCNNCACGNGNACNACGGCGCGANGGGCNCGCCCGGGGCGGGCGGCGGGCGCGGCNNCCAGANGCAGCCGCAACACCAGCACCAGCACCAGCACCAGCACCACCCCCCGGGCCCCGGACAGCCGNCGCCCCCNCCGGAGGCACNGCCCNGCCGGGACGGCACGGACCCCAGNCAGCCCGCCGAGCNCCNCGGGGAGGNGGACCGCACGGAANNNGAACAGNANCNGCACNNCGNGNGCAAGCCNGAGANGGGCCNCCCCNACCAGGGGCANGACNCCGGNGNGAANCNCCCCGACAGCCACGGGGCCANNNCCNCGGNGGNGNCCGACGCCAGCNCCGCGGNANANNACNGCAACNANCCNGACGNGNAGACNAGNGAGCNCGCNNNCNNGCNGNCCAANNNCNANNAAAGGNNCCNNNGNNCCCNAAGNCCAACNACNAAACNGGGGGANANNANGAAGGGCCNNGAGCANCNGGANNCNGCCNAANAAAAAACANNNANNNNCANNGCAA

SEQ ID NO: 4 - DNA, 1167 bp, Homo sapien
atggcttcgctgctgggagcctacccttggcccgagggtctcgagtgcccggccctggacgccgagctgtcggatggacaatcgccgccggccgtcccccggcccccgggggacaagggctccgagagccgtatccggcggcccatgaacgccttcatggtttgggccaaggacgagaggaaacggctggcagtgcagaacccggacctgcacaacgccgagctcagcaagatgctgggaaagtcgtggaaggcgctgacgctgtcccagaagaggccgtacgtggacgaggcggagcggctgcgcctgcagcacatgcaggactaccccaactacaagtaccggccgcgcaggaagaagcaggccaagcggctgtgcaagcgcgtggacccgggcttccttctgagctccctctcccgggaccagaacgccctgccggagaagagaagcggcagccggggggcgctgggggagaaggaggacaggggtgagtactcccccggcactgccctgcccagcctccggggctgctaccacgaggggccggctggtggtggcggcggcggcaccccgagcagtgtggacacgtacccgtacgggctgcccacacctcctgaaatgtctcccctggacgtgctggagccggagcagaccttcttctcctccccctgccaggaggagcatggccatccccgccgcatcccccacctgccagggcacccgtactcaccggagtacgccccaagccctctccactgtagccaccccctgggctccctggcccttggccagtcccccggcgtctccatgatgtcccctgtacccggctgtcccccatctcctgcctattactccccggccacctaccacccactccactccaacctccaagcccacctgggccagctttccccgcctcctgagcaccctggcttcgacgccctggatcaactgagccaggtggaactcctgggggacatggatcgcaatgaattcgaccagtatttgaacactcctggccacccagactccgccacaggggccatggccctcagtgggcatgttccggtctcccaggtgacaccaacgggtcccacagagaccagcctcatctccgtcctggctgatgccacggccacgtactacaacagctacagtgtgtcatag

SEQ ID NO: 5 - DNA, 1155 bp, Homo sapien
atgcagagatcgccgcccggctacggcgcacaggacgacccgcccgcccgccgcgactgtgcatgggccccgggacacggggccgccgctgacacgcgcggcctcgccgccggccccgccgccctcgccgcgcccgccgcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggctag

SEQ ID NO: 6 - DNA, 6852 bp, Artificial
  Sox17 in XLone plasmid
caggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatcttatcgtcatcgtctttgtaatccatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccactcgagtggaattatcacctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaaggtaccgccaccatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtagactagtagaccacctcccctgcgagctaagctggacagccaatgacgggtaagagagtgacatttttcactaacctaagacaggagggccgtcagagctactgcctaatccaaagacgggtaaaagtgataaaaatgtatcactccaacctaagacaggcgcagcttccgagggatttgagatccagacatgataagatacattgatgagtttggacaaaccaaaactagaatgcagtgaaaaaaatgccttatttgtgaaatttgtgatgctattgccttatttgtaaccattataagctgcaataaacaagtttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaggtcgacttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttagcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctggagatttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggggccggggttctcctccacgtcgccggcctgcttcagcaggctgaagttggtggcgccgctgcccccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgaca

SEQ ID NO: 7 - DNA, 6774 bp, Artificial
  Sox7 in XLone plasmid
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaaggtaccgccaccatggcttcgctgctgggagcctacccttggcccgagggtctcgagtgcccggccctggacgccgagctgtcggatggacaatcgccgccggccgtcccccggcccccgggggacaagggctccgagagccgtatccggcggcccatgaacgccttcatggtttgggccaaggacgagaggaaacggctggcagtgcagaacccggacctgcacaacgccgagctcagcaagatgctgggaaagtcgtggaaggcgctgacgctgtcccagaagaggccgtacgtggacgaggcggagcggctgcgcctgcagcacatgcaggactaccccaactacaagtaccggccgcgcaggaagaagcaggccaagcggctgtgcaagcgcgtggacccgggcttccttctgagctccctctcccgggaccagaacgccctgccggagaagagaagcggcagccggggggcgctgggggagaaggaggacaggggtgagtactcccccggcactgccctgcccagcctccggggctgctaccacgaggggccggctggtggtggcggcggcggcaccccgagcagtgtggacacgtacccgtacgggctgcccacacctcctgaaatgtctcccctggacgtgctggagccggagcagaccttcttctcctccccctgccaggaggagcatggccatccccgccgcatcccccacctgccagggcacccgtactcaccggagtacgccccaagccctctccactgtagccaccccctgggctccctggcccttggccagtcccccggcgtctccatgatgtcccctgtacccggctgtcccccatctcctgcctattactccccggccacctaccacccactccactccaacctccaagcccacctgggccagctttccccgcctcctgagcaccctggcttcgacgccctggatcaactgagccaggtggaactcctgggggacatggatcgcaatgaattcgaccagtatttgaacactcctggccacccagactccgccacaggggccatggccctcagtgggcatgttccggtctcccaggtgacaccaacgggtcccacagagaccagcctcatctccgtcctggctgatgccacggccacgtactacaacagctacagtgtgtcatagactagtagaccacctcccctgcgagctaagctggacagccaatgacgggtaagagagtgacatttttcactaacctaagacaggagggccgtcagagctactgcctaatccaaagacgggtaaaagtgataaaaatgtatcactccaacctaagacaggcgcagcttccgagggatttgagatccagacatgataagatacattgatgagtttggacaaaccaaaactagaatgcagtgaaaaaaatgccttatttgtgaaatttgtgatgctattgccttatttgtaaccattataagctgcaataaacaagtttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaggtcgacttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttagcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctggagatttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggggccggggttctcctccacgtcgccggcctgcttcagcaggctgaagttggtggcgccgctgcccccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatcttatcgtcatcgtctttgtaatccatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccactcgagtggaattatcacctc

SEQ ID NO: 8 - DNA, 6762 bp, Artificial
  Sox18 in XLone plasmid
tccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaaggtaccgccaccatgcagagatcgccgcccggctacggcgcacaggacgacccgcccgcccgccgcgactgtgcatgggccccgggacacggggccgccgctgacacgcgcggcctcgccgccggccccgccgccctcgccgcgcccgccgcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggctagactagtagaccacctcccctgcgagctaagctggacagccaatgacgggtaagagagtgacatttttcactaacctaagacaggagggccgtcagagctactgcctaatccaaagacgggtaaaagtgataaaaatgtatcactccaacctaagacaggcgcagcttccgagggatttgagatccagacatgataagatacattgatgagtttggacaaaccaaaactagaatgcagtgaaaaaaatgccttatttgtgaaatttgtgatgctattgccttatttgtaaccattataagctgcaataaacaagtttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaggtcgacttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttagcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctggagatttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggggccggggttctcctccacgtcgccggcctgcttcagcaggctgaagttggtggcgccgctgcccccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatcttatcgtcatcgtctttgtaatccatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccactcgagtggaattatcacctcgagtttac

SEQ ID NO: 9 - DNA, 2722 bp, Homo sapien
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggaggaagacacagcaccctggagatgttaaggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggattaaatgagttagcctgcagaaatcacttgatgcacagcacatgggaagcattgtgtgtatttattaatccttcacaaagtctttgagatatatttttatcaaatatttagcatggatcccggtacactttcaatacttaataaatggtcaatgttattctttttcactatt

SEQ ID NO: 10 - protein, 250 aa, Homo sapien
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQEEDTAPWRC

SEQ ID NO: 11 - protein, 388 aa, Homo sapien [sequence omitted]

SEQ ID NO: 12 - protein, 384 aa, Homo sapien [sequence omitted]

SEQ ID NO: 13 - DNA, 20 bp, Artificial
  GAPDHFWD Primer
gtggacctgacctgccgtct

SEQ ID NO: 14 - DNA, 20 bp, Artificial
  GAPDH_REV Primer
ggaggagtgggtgtcgctgt

SEQ ID NO: 15 - DNA, 19 bp, Artificial
  OCT4_FWD Primer
cagtgcccgaaacccacac

SEQ ID NO: 16 - DNA, 20 bp, Artificial
  OCT4_REV Primer
ggagacccagcagcctcaaa

SEQ ID NO: 17 - DNA, 20 bp, Artificial
  SOX2_FWD Primer
caagatgcacaactcggaga

SEQ ID NO: 18 - DNA, 20 bp, Artificial
  SOX2_REV Primer
gttcatgtgcgcgtaactgt

SEQ ID NO: 19 - DNA, 18 bp, Artificial
  SOX17_FWD Primer
ggcgcagcagaatccaga

SEQ ID NO: 20 - DNA, 18 bp, Artificial
  SOX17_REV Primer
ccacgacttgcccagcat

SEQ ID NO: 21 - DNA, 19 bp, Artificial
  GATA2_FWD Primer
ctcccagctctactccagg

SEQ ID NO: 22 - DNA, 18 bp, Artificial
  GATA2_REV Primer
gtggtgtgagtcggggtg

SEQ ID NO: 23 - DNA, 20 bp, Artificial
  CD31_FWD Primer
gctgacccttctgctctgtt

SEQ ID NO: 24 - DNA, 20 bp, Artificial
  CD31_REV Primer
tgagaggtggtgctgacatc

SEQ ID NO: 25 - DNA, 20 bp, Artificial
  VEC_FWD Primer
gtgttcacgcatcggttgtt

SEQ ID NO: 26 - DNA, 23 bp, Artificial
  VEC_REV Primer
caaatgtgtacttggtctgggtg

SEQ ID NO: 27 - DNA, 22 bp, Artificial
  CD34_FWD Primer
cctaagtgacatcaaggcagaa

SEQ ID NO: 28 - DNA, 19 bp, Artificial
  CD34_REV Primer
gcaaggagcagggagcata

SEQ ID NO: 29 - DNA, 20 bp, Artificial
  DLL4_FWD Primer
gcactccctggcaatgtact

SEQ ID NO: 30 - DNA, 20 bp, Artificial
  DLL4_REV Primer
gccatcctcctggtccttac

SEQ ID NO: 31 - DNA, 21 bp, Artificial
  TAL1_FWD Primer
gggatctgattctatcgccca

SEQ ID NO: 32 - DNA, 20 bp, Artificial
  TAL1_REV Primer
gctttccccctttttcgctg

SEQ ID NO: 33 - DNA, 19 bp, Artificial
  SPI1_FWD Primer
gccctgcaatgtcaaggga

SEQ ID NO: 34 - DNA, 21 bp, Artificial
  SPI1_REV Primer
gaagtcccagtaatggtcgct

SEQ ID NO: 35 - DNA, 19 bp, Artificial
  ETS1_FWD Primer
catccacaagacagcgggg

SEQ ID NO: 36 - DNA, 19 bp, Artificial
  ETS1_REV Primer
ctcgtcggcatctggcttg

SEQ ID NO: 37 - DNA, 20 bp, Artificial
  RUNX1_FWD Primer
ggtttcgcagcgtggtaaaa

SEQ ID NO: 38 - DNA, 20 bp, Artificial
  RUNX1_REV Primer
gcactgtgggtacgaaggaa

SEQ ID NO: 39 - DNA, 20 bp, Artificial
  CD43_FWD Primer
cattcctcagccaagagcca

SEQ ID NO: 40 - DNA, 19 bp, Artificial
  CD43_REV Primer
gcaagagctgggacctgag

SEQ ID NO: 41 - DNA, 20 bp, Artificial
  CD45_FWD Primer
caacagtggagaaaggacgc

SEQ ID NO: 42 - DNA, 20 bp, Artificial
  CD45_REV Primer
tgtccagaaaggcaaagcca

SEQ ID NO: 43 - DNA, 20 bp, Artificial
  LCOR_FWD Primer
cagcaatgctccgttgagag

SEQ ID NO: 44 - DNA, 20 bp, Artificial
  LCOR_REV Primer
gagggaaacgggacctacac

SEQ ID NO: 45 - DNA, 20 bp, Artificial
  ERG_FWD Primer
cttggtcggaatggggagag

SEQ ID NO: 46 - DNA, 21 bp, Artificial
  ERG_REV Primer
tgttcagaacctgacggcttt

SEQ ID NO: 47 - DNA, 23 bp, Artificial
  HOXA5_FWD Primer
gctgcacataagtcatgacaaca

SEQ ID NO: 48 - DNA, 20 bp, Artificial
  HOXA5_REV Primer
cgctcagatactcagggacg

SEQ ID NO: 49 - DNA, 20 bp, Artificial
  HOXA9_FWD Primer
cccatcgatcccaataaccc

SEQ ID NO: 50 - DNA, 20 bp, Artificial
  HOXA9_REV Primer
tgtggcctgaggtttagagc

SEQ ID NO: 51 - DNA, 20 bp, Artificial
  HOXA10_FWD Primer
tacttccgcctttctcaggc

SEQ ID NO: 52 - DNA, 20 bp, Artificial
  HOXA10_REV Primer
cctttggaattgcccaggga